(19) United States
(12) Patent Application Publication
(10) Pub. No.: US 2026/0193250 A1
(43) Pub. Date: Jul. 9, 2026

(54) TOLL-LIKE RECEPTOR 7 AGONISTS AS IMMUNE-STIMULATORS TO ELICIT THE INNATE ANTITUMOR IMMUNITY

(71) Applicant: Merck Patent GmbH, Darmstadt (DE)

(72) Inventors: Min Shan, Darmstadt (DE); Jean Wakim, Darmstadt (DE); Andreas Goutopoulos, Rockland, MA (US)

(73) Assignee: Merck Patent GmbH, Darmstadt (DE)

(21) Appl. No.: 19/128,206

(22) PCT Filed: Nov. 8, 2023
(86) PCT No.: PCT/EP2023/081105

§ 371 Date: May 8, 2025

(30) Foreign Application Priority Data
Nov. 9, 2022 (EP) 22206447.9

(51) Int. Cl. C07D 471/04; A61K 9/08; A61K 31/444; A61K 31/4545; A61K 31/4745; A61K 31/496; A61K 31/5377; A61K 38/21; A61K 39/00; A61K 39/39; A61K 47/10; A61K 47/12; A61K 47/14; A61K 47/20; A61N 5/06

(52) U.S. Cl. CPC C07D 471/04; A61K 9/08; A61K 31/444; A61K 31/4545; A61K 31/4745; A61K 31/496; A61K 31/5377; A61K 38/217; A61K 39/39; A61K 47/10; A61K 47/12; A61K 47/14; A61K 47/20; A61N 5/062; A61K 2039/55511

(57) ABSTRACT

Compounds can specifically activate TLR7. The compounds are useful because they can stimulate innate immunity. The compounds can be used in the treatment of conditions including cancer, viral infections and skin lesions. The compounds can optionally be formulated for enhanced penetration following topical administration, the composition preferably initiating a local specific inflammatory cytokine response while limiting undesirable erythema and other inflammatory reactions. Pharmaceutical compositions contain the compound and preferably an additional compound such as imiquimod and/or resiquimod (R848). A composition contains the compound and a compound can be used as a medicament. Other aspects, embodiments, advantages and applications will become clear from the further description herein.

### 1 FIELD OF THE INVENTION

The present invention relates to compounds that can specifically activate TLR7. The compounds of the invention are useful because they can stimulate innate immunity. The compounds of the invention can be used in the treatment of conditions including cancer, viral infections and skin lesions. The compounds can optionally be formulated for enhanced penetration following topical administration, said composition preferably initiating a local specific inflammatory cytokine response while limiting undesirable erythema and other inflammatory reactions. The invention also relates to pharmaceutical compositions comprising the compound of the invention and preferably an additional compound such as imiquimod and/or resiquimod (R848). The invention also relates to a composition comprising a compound of the invention and to a compound for use as a medicament. Other aspects, embodiments, advantages and applications of the invention will become clear from the further description herein.

### 2 BACKGROUND OF THE INVENTION

Programmed cell death protein 1, also known as PD-1 and CD279 (cluster of differentiation 279), is a protein on the surface of T and B cells that has a role in regulating the immune system's response to the cells of the human body by down-regulating the immune system and promoting self-tolerance by suppressing T cell inflammatory activity. This prevents autoimmune diseases, but it can also prevent the immune system from killing cancer cells. The role of the human immune system in the antitumor field was underestimated for many years until the successful clinical application of immune checkpoint inhibitors, e.g. inhibitors of PD-1/PD-L1. PD-1 inhibitors and PD-L1 inhibitors are a group of checkpoint inhibitor drugs that block the activity of PD-1 and PDL1 immune checkpoint proteins present on the surface of cells.

The immune system has multiple “brake” mechanisms to negatively down-regulate the activation and function of T cells or other immune cells to protect the host's own health cells. Check point inhibitors like PD-1/PD-L1 as well as CTLA-4 antagonists can selectively release “brakes” of the immune system to leverage the so-called “adaptive immunity” and produce durable antitumor responses. However, only a small population of cancer patients typically benefits clinically when using check point inhibitors.

The human immune system is also designed to protect the body from foreign entities like virus and bacteria through a systemic coordination between a plethora of cell types with high-selective immunologic functions, the so-called “innate immunity”. Cell types include phagocytic cell like dendritic cells (DCs), macrophages, gamma delta T cells, and natural killer (NK) cells act as barriers against foreign entities. The mechanism behind this is to utilize conserved molecular patterns of pathogens and expressed Fc receptor for recognizing and initiating a rapid immune response. Since B and T cells from the adaptive immune system are able to mount a memory response, a communication between the innate and adaptive immune systems mediated through DCs can initiate a specific and durable immunologic response.

DCs are responsible for constantly sampling their environment through phagocytosis, receptor-mediated endocytosis, and micropinocytosis. With the help of pattern recognition receptors (PRRs) DCs are able to recognize a plethora of foreign entities. Once PRRs engage, DCs become activated and mature into stronger antigen presenting cells (APCs) and upregulate cell surface expression of co-stimulatory molecules like CD40 and CD86 to achieve optimal T cell priming and activation.

Tool-like receptors (TLRs) are well known as a member of PRR family. Toll-like receptors (TLR) currently comprising a gene family of 10 receptors with different specificities are part of the cellular pathogen pattern recognition system, which has evolved for defense against a variety of infections (bacteria, virus, fungi). Activation of TLRs leads to cytokine responses, e.g. with release of interferons and activation of specified immune cells. The functional expression of selected TLRs in tissues is highly different. Part of the receptors are located at the cell surface such as TLR4 (stimulated by E. coli lipopolysaccharide LPS), e.g. on epithelial cells, or TLR3, 7, 8 and 9 located at endosomal membranes in specified immune cells. The latter are all activated by nucleic acids, but recognize various types of them. For instance, TLR9 is activated by single stranded DNA containing CpG subsequences, TLR7 and 8 are activated by single stranded RNA, and TLR3 is activated by double-stranded RNA.

TLRs have also been implicated in various autoimmune and inflammatory diseases, with the clearest example being the role played by TLR7 in the pathogenesis of systemic lupus erythematosus (Barrat and Coffman, Immunol Rev, 223:271-283, 2008). Additionally, a TLR8 polymorphism has been associated with rheumatoid arthritis (Enevold et al., J Rheumatol, 37:905-10, 2010).

Each TLR has a specific class of molecules for recognizing different surface and intracellular components of microorganisms, ranging from the membrane lipids of bacteria to single or double stranded RNAs of a virus. TLRs that are expressed intracellularly are in the endosomal compartment and include TLRs such as TLR3, TLR7, TLR8, TLR9, and TLR13. TLR7 and TLR8 are reported to be dimeric TLR receptors that comprise a leucine-rich repeat (LRR) motif as an ectodomain, a transmembrane domain, and a cytoplasmic domain, which has a Toll/interleukin-1 (IL-1) receptor (TIR) signaling domain. It has been reported that TLR7 is expressed in B cells and plasmacytoid dendritic cells (pDCs), while TLR8 can be expressed in monocytes and myeloid dendritic cells (mDCs).

Upon an agonistic binding on TLR7/8 with a degradation product like guanosine derived from single stranded RNAs (ssRNA), the dimerization interface is rearranged including both the protein-protein and ligand-mediated interfaces. A conformational change in TLR7/8 ectodomains leads to an activated configuration of the cytoplasmic TIR signaling domains. Subsequently, there is a MyD88 dependent signal followed by a strong interferon response and pro-inflammatory cytokine production in DCs. Moreover, activated DCs also upregulate cell surface expression of co-stimulatory molecules like CD40 and CD86. The co-stimulatory expression and engagement leads to optimal T cell priming and activation.

More recently, it was found that the tumor associated antigen (TAA) presentation has an impact on priming and activation of T cells to generate a robust, long-term antitumor immune response. Studies showed that antigen uptake and presentation by APCs is more efficient when antigen is bound in an antibody-antigen complex, the so called “immune complex”. Since activated DCs also upregulate the expression of major histocompatibility complex (MHC), the antigen internalized during an encounter of a microorganism is digested and its peptides are displayed by the MHC on the cell membrane of DCs. Activated T cells can recognize and kill tumor cells containing MHC-bound neoantigens.

As mentioned above, only a small population of patients experience clinical benefits with check point inhibitors. For cancer patients one reason for this is that immune cell presence and frequency can differ from tumor to tumor. A low T cell frequency in the tumor immune microenvironment (TIME) is considered as “cold”, while a high T cell frequency as “hot”. Clinical studies revealed that patients with hot tumors had stronger outcomes and longer disease-free survival than those with cold.

Although an agonistic stimulatory via the TLR7/8 pathway can activate DCs followed by T cell priming and activation, small synthetic molecules like imiquimod and resiquimod (R848) have only been approved as a topical treatment, for example, imiquimod for the treatment of basal cell carcinoma via intratumoral to avoid systemic inflammation. Such a clinical treatment largely limits the TLR7/8 agonist therapy for solid tumors.

When cancerous lesions progress, they may become metastatic and chemotherapy, radiation therapy and immunotherapy may be used in addition to surgery. Resiquimod, a member of the imidazoquinoline family related in structure to imiquimod, is an immune response modifier which acts as a Toll-like receptor 7 and 8 agonist. However, it is distinctively different from Imiquimod since Imiquimod signals through the toll-like receptor 7 (TLR7) only. Imiquimod is FDA approved for the treatment of a number of skin diseases. Compared with imiquimod, resiquimod is a more potent inducer of TNF-α, IL-1, IL-6, IL-8, and IL-12. Resiquimod has been shown to promote cross-presentation of exogenous antigens, resulting in the efficient induction of antigen-specific CD8+ T-cell responses in an animal model. Results from animal studies have confirmed the ability of resiquimod to activate dendritic cells, including the capacity to induce local activation of immune cells, stimulate the production of proinflammatory cytokines, and enhance antigen-presentation by dendritic cells leading to activation of effective cellular responses. Systemic delivery of resiquimod, with radiation, primes durable antitumor immune responses in a lymphoma model (Dovedi S J 2013, Blood 121(2):251-9.). Resiquimod has been used in clinical trials to treat actinic keratosis, cutaneous T cell lymphoma and herpes simplex virus with mixed results. Other prior art uses of resiquimod include administration as a vaccine adjuvant agent to treat various diseases, including metastatic melanoma with inconsistent results. Resiquimod has been used as a vaccine adjuvant for NYES0-1 protein vaccine in treatment of melanoma (Sabado R L Cancer Immunol Res. 2015).

There remains a need for alternative TLR7 agonists that are moreover preferably specific and effective.

### 3 SUMMARY OF THE INVENTION

The present invention aims to provide compounds that are effective TLR7 agonists to targeting innate immunity. The inventors found novel imidazoquinoline-based TLR7 agonists that are for example effective in initiating a specific and durable immunologic response against for example cancer.

Therefore, in a first aspect the invention provides a compound according to formula I

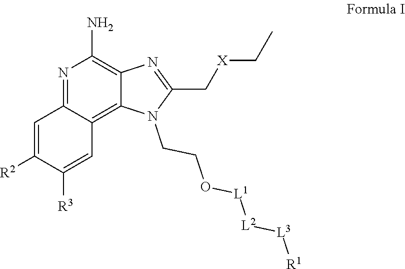

or a pharmaceutically acceptable salt thereof,
wherein X is an oxygen atom, C1-C5-alkyl (preferably CH₂) or NH;
wherein R² and R³ are each independently selected from the group consisting of hydrogen, C1-C5-alkyl, C4-C7-cycloalkyl, C4-C7-heterocycloalkyl, aryl and heteroaryl; preferably both R² and R³ are each hydrogen;
and wherein L¹, L², L³ and R¹ are as defined below under (a) or (b) or (c):
(a) L¹ is C2-C6 alkyl;
L² is a 5- or 6-membered heterocycle or OH; L³ is absent or selected from the group consisting of hydrogen, C1-C2 alkyl and CH₂C(CH₃)₂; and
R¹ is absent or selected from the group consisting of hydrogen, NH₂, OH and SCH₃;
(b) L¹ is C2-C6 alkyl;
L² is selected from the group consisting of C(O), NHC(O)CH₂ and NHC(O)C(CH₃)₂;
L³ is selected from the group consisting of a 6-membered heterocycle, OH, C(O)O, S, SO₂ and SO₃H; and
R¹ is absent or methyl or hydrogen;
(c) L¹ is C(O)CH₂;
L² is a 6-membered heterocycle or NH₂;
L³ is not present; and
R¹ is methyl or hydrogen or absent;
and wherein if L³ is not present then L² is directly bound via a covalent bond to R¹.

In a preferred embodiment of the first aspect R² and R³ are hydrogen.

Preferably, X in the compound of the invention is an oxygen atom.

In a further embodiment, the compound of the invention has a structure according to formula II

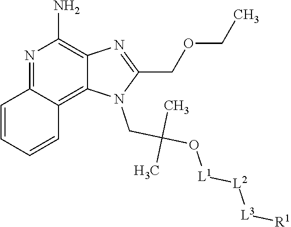

Formula II
wherein L¹, L², L³ and R¹ are as defined below under (d), (e) or (f)
(d) L¹ is C2-C6 alkyl;
L² is a 5- or 6-membered heterocycle (preferably triazole or piperazine), or OH; L³ is absent or selected from the group consisting of hydrogen, C1-C2 alkyl and CH₂C(CH₃)₂; and
R¹ is absent or selected from the group consisting of hydrogen, NH₂, OH and SCH₃;
(e) L¹ is C2-C3 alkyl;
L² is selected from the group consisting of C(O), NHC(O)CH₂ and NHC(O)C(CH₃)₂;
L³ is selected from the group consisting of a 6-membered heterocycle (preferably piperazine), OH, C(O)O, S, SO₂ and SO₃H; and
R¹ is absent or methyl or hydrogen;
(f) L¹ is C(O)CH₂;
L² is a 6-membered heterocycle (preferably piperazine) or NH₂;
L³ is not present; and
R¹ is methyl or hydrogen or absent;

In a preferred embodiment of a compound with Formula II of the invention
L¹ is C2-C6 alkyl;
L² is a 5- or 6-membered heterocycle or OH; L³ is absent or selected from the group consisting of hydrogen, C1-C2 alkyl and CH₂C(CH₃)₂; and
R¹ is absent or selected from the group consisting of NH₂, OH and SCH₃;

In a particularity preferred embodiment of the compound of the invention the compound has a structure selected from the group consisting of the structures listed below that are designated as Example 1 through Example 27:

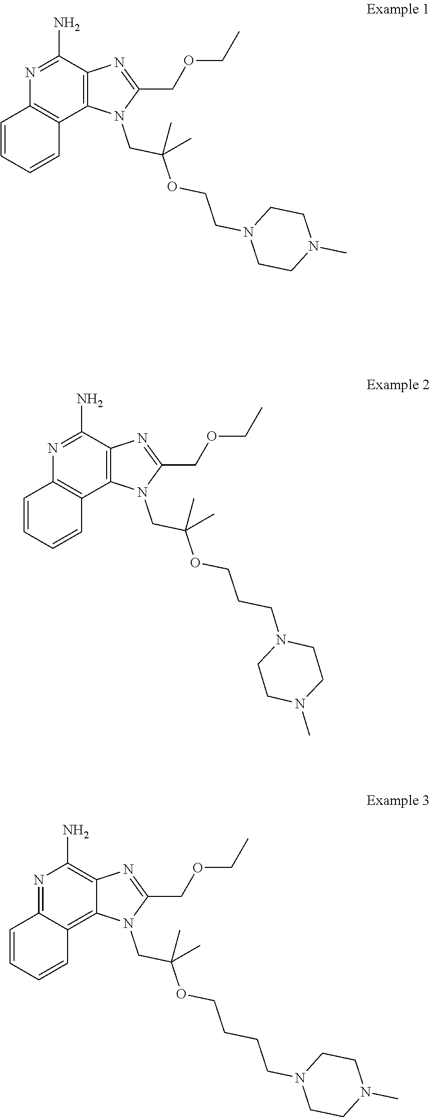 [SMILES: CCOCc1nc2c(N)nc3ccccc3c2n1CC(C)(C)OCCN1CCN(C)CC1 ; CCOCc1nc2c(N)nc3ccccc3c2n1CC(C)(C)OCCCN1CCN(C)CC1 ; CCOCc1nc2c(N)nc3ccccc3c2n1CC(C)(C)OCCCCN1CCN(C)CC1]

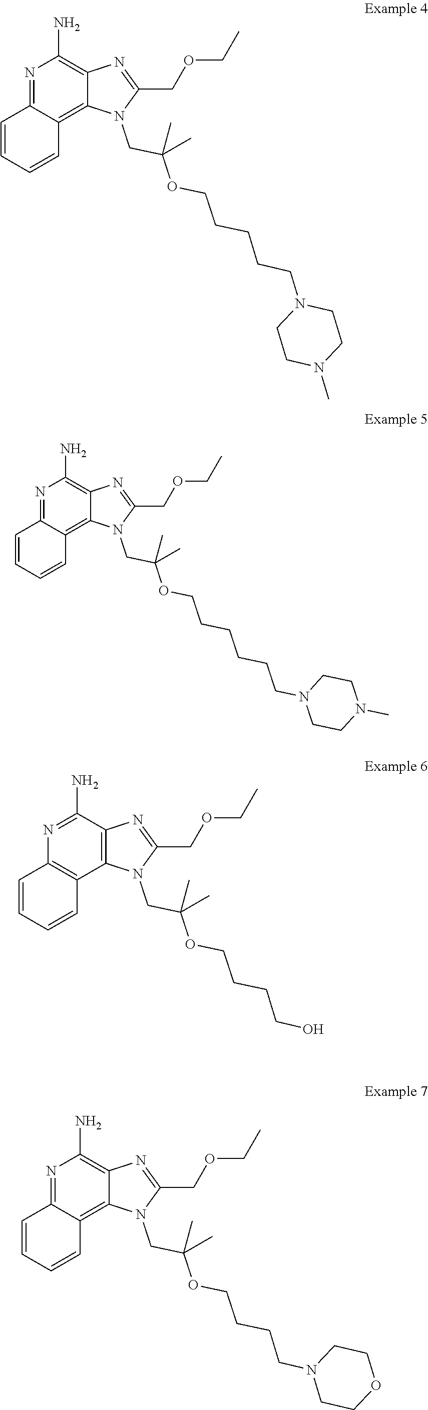 [SMILES: CCOCc1nc2c(N)nc3ccccc3c2n1CC(C)(C)OCCCCCN1CCN(C)CC1 ; CCOCc1nc2c(N)nc3ccccc3c2n1CC(C)(C)OCCCCCCN1CCN(C)CC1 ; CCOCc1nc2c(N)nc3ccccc3c2n1CC(C)(C)OCCCCO ; CCOCc1nc2c(N)nc3ccccc3c2n1CC(C)(C)OCCCCN1CCOCC1]

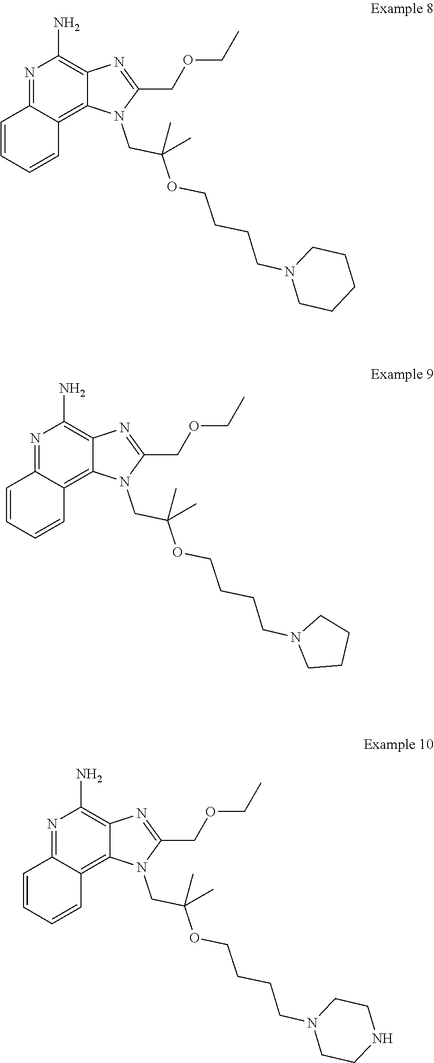 [SMILES: CCOCc1nc2c(N)nc3ccccc3c2n1CC(C)(C)OCCCCN1CCCCC1 ; CCOCc1nc2c(N)nc3ccccc3c2n1CC(C)(C)OCCCCN1CCCC1 ; CCOCc1nc2c(N)nc3ccccc3c2n1CC(C)(C)OCCCCN1CCNCC1]

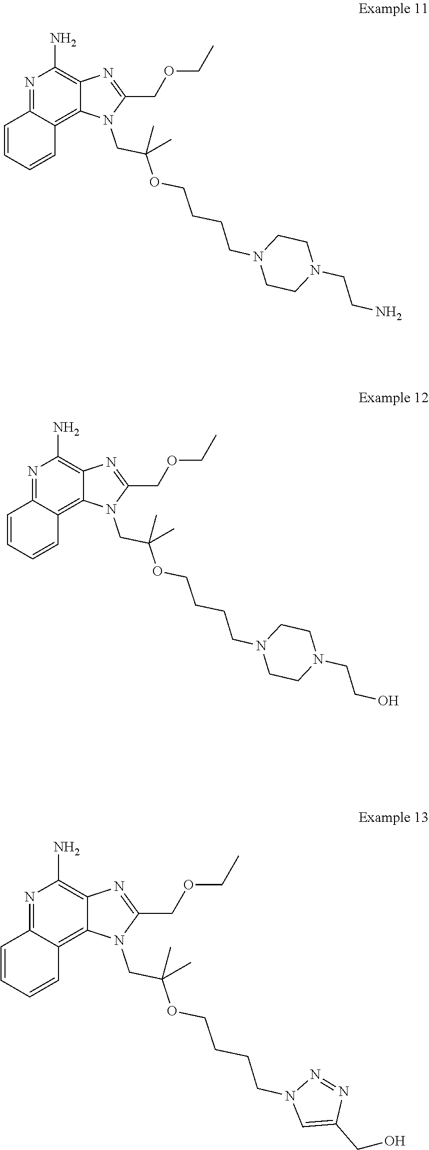 [SMILES: CCOCc1nc2c(N)nc3ccccc3c2n1CC(C)(C)OCCCCN1CCN(CCN)CC1 ; CCOCc1nc2c(N)nc3ccccc3c2n1CC(C)(C)OCCCCN1CCN(CCO)CC1 ; CCOCc1nc2c(N)nc3ccccc3c2n1CC(C)(C)OCCCCn1cc(CO)nn1]

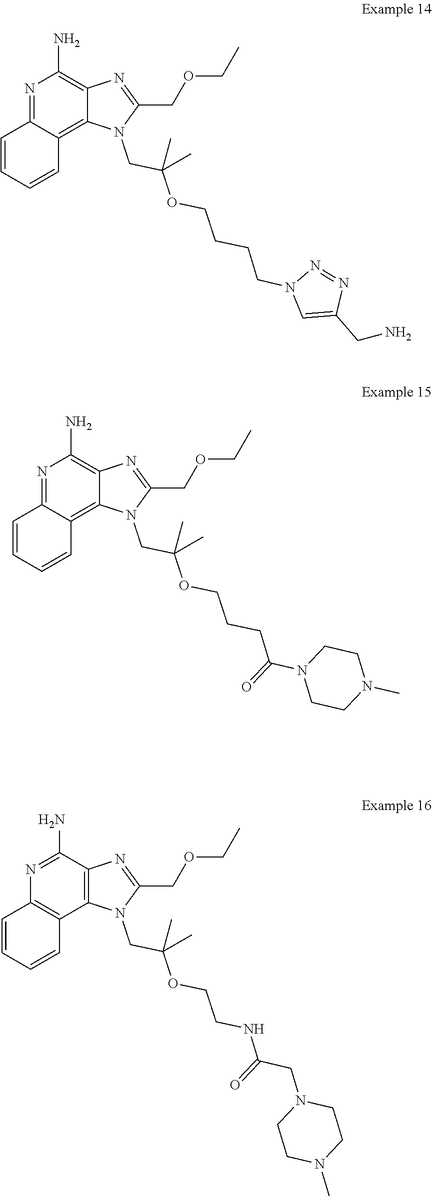 [SMILES: CCOCc1nc2c(N)nc3ccccc3c2n1CC(C)(C)OCCCCn1cc(CN)nn1 ; CCOCc1nc2c(N)nc3ccccc3c2n1CC(C)(C)OCCCC(=O)N1CCN(C)CC1 ; CCOCc1nc2c(N)nc3ccccc3c2n1CC(C)(C)OCCNC(=O)CN1CCN(C)CC1]

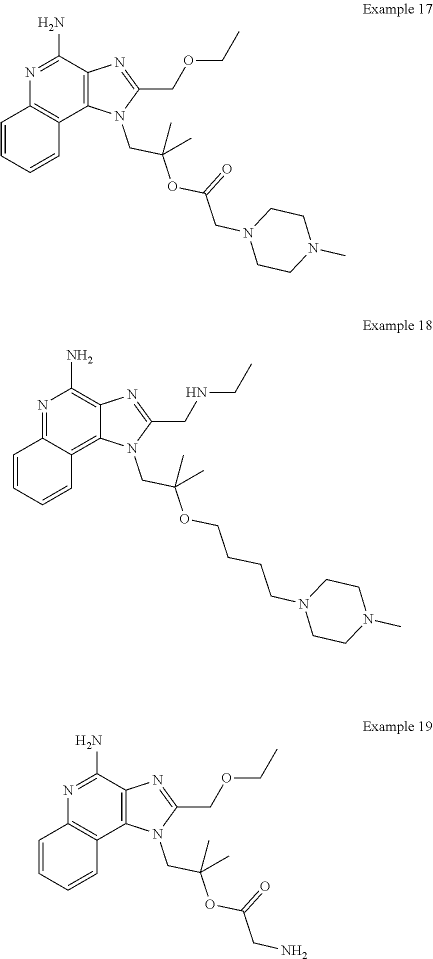 [SMILES: CCOCc1nc2c(N)nc3ccccc3c2n1CC(C)(C)OC(=O)CN1CCN(C)CC1 ; CCNCc1nc2c(N)nc3ccccc3c2n1CC(C)(C)OCCCCN1CCN(C)CC1 ; CCOCc1nc2c(N)nc3ccccc3c2n1CC(C)(C)OC(=O)CN]

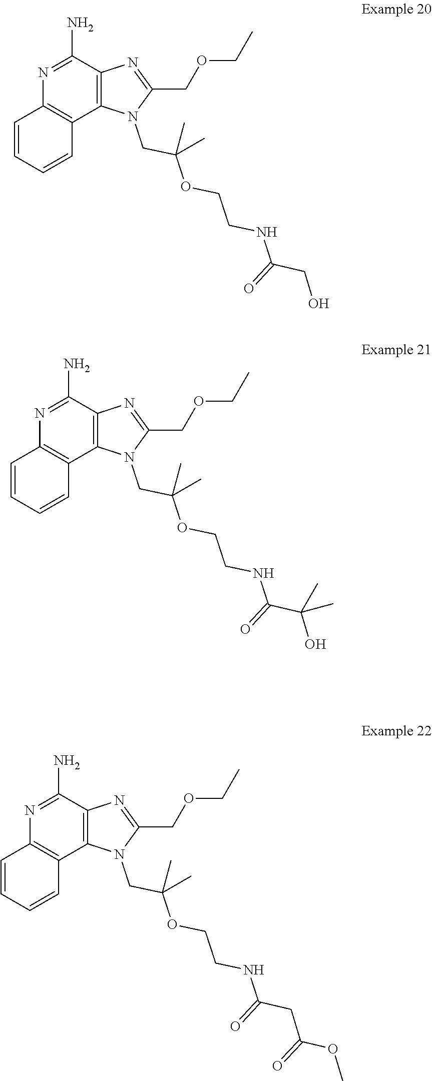 [SMILES: CCOCc1nc2c(N)nc3ccccc3c2n1CC(C)(C)OCCNC(=O)CO ; CCOCc1nc2c(N)nc3ccccc3c2n1CC(C)(C)OCCNC(=O)C(C)(C)O ; CCOCc1nc2c(N)nc3ccccc3c2n1CC(C)(C)OCCNC(=O)CC(=O)OC]

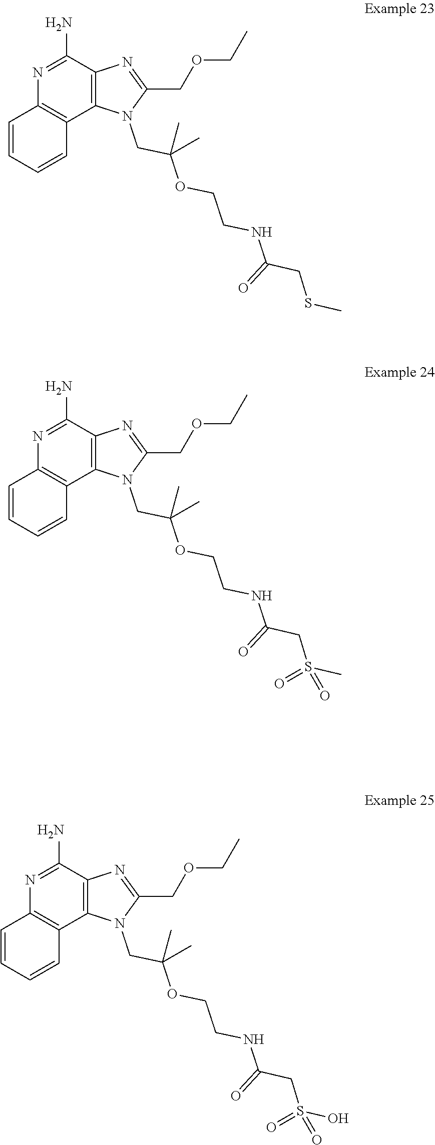 [SMILES: CCOCc1nc2c(N)nc3ccccc3c2n1CC(C)(C)OCCNC(=O)CSC ; CCOCc1nc2c(N)nc3ccccc3c2n1CC(C)(C)OCCNC(=O)CS(C)(=O)=O ; CCOCc1nc2c(N)nc3ccccc3c2n1CC(C)(C)OCCNC(=O)CS(=O)(=O)O]

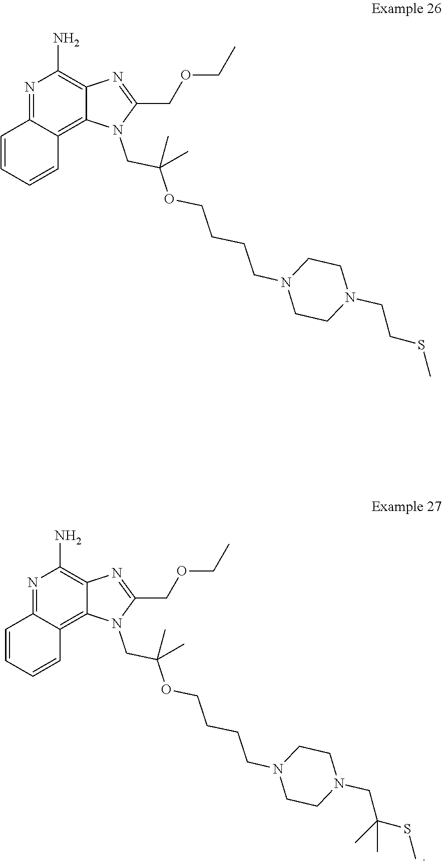 [SMILES: CCOCc1nc2c(N)nc3ccccc3c2n1CC(C)(C)OCCCCN1CCN(CCSC)CC1 ; CCOCc1nc2c(N)nc3ccccc3c2n1CC(C)(C)OCCCCN1CCN(CC(C)(C)SC)CC1]

A further preferred compound of the invention relates to a compound wherein
X is NH;
L¹ is C2-C6 alkyl;
L² is a 5- or 6-membered heterocycle;
L³ is not present; and
R¹ is methyl or hydrogen; and
R² and R³ are each hydrogen.

Also preferred is a compound of the invention wherein the compound
(i) activates TLR7 more strongly than TLR8; and/or
(ii) induces IL-6, IL1-b and TNF-α production; and/or
(iii) induces upregulation of CD40 and/or CD86 in peripheral blood mononuclear cells (PBMC).

Also preferred is a compound according to the invention, wherein the compound induces TNFα secretion from peripheral blood mononuclear cells (PBMC) if the compound contacts said cells. Also preferred is a compound according to the invention, wherein the compound has for TLR7 an EC50 of less than 10 μM and preferably less than 0.01 μM, when tested in a test system described in Example 6.2.1.

A further aspect of the invention relates to a pharmaceutical composition comprising a compound of the invention.

The invention also relates to a pharmaceutical composition comprising the compound of the invention and preferably an additional compound such as imiquimod and/or resiquimod (R848). A further aspect of the invention relates to a compound of the invention for the use as a medicament.

A further aspect of the invention relates to a compound of the invention for the use in the treatment of a disease wherein said compound is used in said treatment in combination with the further compounds imiquimod and/or resiquimod (R848).

Preferred embodiments provide a compound of the invention for the use in the treatment of a condition selected from the group consisting of cancer, a viral infection, non-cancerous skin lesions, pre-cancerous skin lesions, cancerous skin lesions, bladder cancer, virally mediated skin disease, said composition optionally being formulated for enhanced penetration following topical administration, said composition preferably initiating a local specific inflammatory cytokine response while limiting undesirable erythema and other inflammatory reactions.

In preferred embodiments of the composition of the invention the compound of the invention is comprised in an amount of between 0.01%-1%.

A further aspect relates to a composition of the invention for use in the treatment of bladder cancer, wherein the composition is formulated for intracystic administration.

In preferred embodiments of the composition of the invention the invention further comprises additional components of one of the groups i through xiii outlined below:
i. Oleic acid (50%) and isopropyl myristate (50%) in a solution containing 50 ml phosphate buffered saline and 50 ml ethanol at a total concentration of 1% wt/vol, or
ii. Isopropyl myristate (50%) and sodium lauryl sulfate (50%) in a solution containing 50 ml phosphate buffered saline and 50 ml ethanol at a total concentration of 0.5% wt/vol, or iii. Oleic acid (50%) and sodium lauryl sulfate (50%) in a solution containing 50 ml phosphate buffered saline and 50 ml ethanol at a total concentration of 0.5% wt/vol, or iv. Oleic acid (33%), isopropyl myristate (33%) and sodium lauryl sulfate (33%) in a solution containing 50 ml phosphate buffered saline and 50 ml ethanol at a total concentration of 0.5% wt/vol, or
v. Isopropyl palmitate (50%) and sodium oleate (50%) in a solution containing 50 ml phosphate buffered saline and 50 ml ethanol at a total concentration of 2% wt/vol, or
vi. Sodium lauryl sulfate (25%) and linoleic acid (75%) in a solution containing 50 ml phosphate buffered saline and 50 ml ethanol at a total concentration of 1.0% wt/vol, or vii. Palmitic acid (50%) and isopropyl laurate (50%) in a solution containing 50 ml phosphate buffered saline and 50 ml ethanol at a total concentration of 2.0% wt/vol, or
viii. Oleic acid (50%) and linoleic acid (50%) in a solution containing 50 ml phosphate buffered saline and 50 ml ethanol at a total concentration of 1.5% wt/vol, or
ix. Linoleic acid (25%), oleic acid (25%) and isopropyl linoleate (50%) in a solution containing 50 ml phosphate buffered saline and 50 ml ethanol at a total concentration of 0.5% wt/vol, or
x. Sodium oleate (33%), oleic acid (33%) and methyl palmitate (33%) in a solution containing 50 ml phosphate buffered saline and 50 ml ethanol at a total concentration of 2.0% wt/vol, or
xi. A solution containing 50 ml phosphate buffered saline and 50 ml ethanol, or
xii. Oleic acid (10%) in a solution containing 50 ml phosphate buffered saline and 50 ml ethanol, or xiii. Oleic acid (2%) and sodium lauryl sulfate (5%) in a solution containing 50 ml phosphate buffered saline and 50 ml ethanol.

A further aspect of the invention relates to a method for the treatment of treatment of a cancer, comprising administering a therapeutically effective amount of the compound of the invention to the subject suffering from cancer.

Yet a further aspect of the invention provides a method for the treatment of skin tumor lesion and virus induced skin disease, comprising topical administration of the composition of the invention, said composition preferably being effective to reduce or eliminate said lesion or disease while limiting adverse skin reactions selected from erythema and inflammation, said composition preferably reducing tumor lesion penetration into surrounding tissue and metastasis to lymph nodes.

Yet a further aspect of the invention relates to a method for the treatment of bladder tumor, comprising intracystic administration of the composition of the invention, said composition preferably enhancing permeation and delivery of the compound of the invention to bladder epithelium while limiting irritation, said composition preferably reducing tumor invasion into surrounding muscular tissue and metastasis to lymph nodes.

In preferred embodiments of the method of the invention, said treatment further comprising systemic administration to said subject of at least one immune modulator selected from anti-PD1, anti-PD-L1, Anti-CTLA-4 antibody, anti-CD137 antibody, agonistic CD40 antibody, CD134 (Anti-OX40) agonist and PLX3397.

In preferred embodiments of the method of the invention, said treatment further comprising systemic administration to said subject of interferon gamma.

In preferred embodiments of the method of the invention, said treatment further comprising administration of local radiation with or without systemic anti-PD1 antibody.

In preferred embodiments of the method of the invention, said treatment further comprising of administration of photodynamic therapy.

In a further aspect the invention provides a method of activating TLR 7 and/or 8 in a biological sample, comprising contacting said biological sample with a compound according to the invention.

In a further aspect the invention provides a compound of the invention for use as a vaccine adjuvant or for use in the treatment of cancer in combination with an anti-cancer immunotherapy agent (preferably ipilimumab, nivolumab, or pembrolizumab).

Other aspects, advantages, applications and uses of the polypeptides and compositions will become clear from the further disclosure herein. Several documents are cited throughout the text of this specification. Nothing herein is to be construed as an admission that the invention is not entitled to antedate such disclosure by virtue of prior invention.

### 4 FIGURE LEGENDS

FIG. 1: FIG. 1. The molecular binding between R848 and monkey TLR7 (PDB 5GMH), in which the unburied hydrogen group of R848 was high-lighted with the black arrow. Top right: The chemical structure of R848.

FIG. 2: The chemical structure of recently reported TLR7/8 agonists.

FIG. 3: A library of high-potent and selective TLR7 agonists.

FIG. 4: Testing TLR7 agonsists in vitro. An X-Y scatter plot was generated for each agonist/stimulant by plotting the concentration of the agonist vs the median fluorescence intensity of the CD86 stain within a cDC population (FIG. 4A) and a pDC population (FIG. 4B) and monocytes (FIG. 4C) using Graph Pad Prism9 software.

### 5 DETAILED DESCRIPTION

Skin cancer is usually superficially located. Melanoma in situ is still confined to the epidermis and typically grows slowly. Such lesions may take years to progress to invasive melanoma. Invasive melanoma usually has an epidermal component but is no longer confined to the epidermis as melanoma cells invade the dermis. Metastatic cancer is usually deeply located. Data was shown in WO 2017/004421 (incorporated herein by reference) demonstrating that resiquimod functions in multiple in vivo pigmented lesion transgenic mouse models and in a syngeneic melanoma mouse model which have an intact immune system. These data showed that resiquimod was effective in inhibiting or killing melanocytic tumor cells in nevus, atypical intraepidermal melanocytic proliferation and melanoma. In addition, the mice treated with resiquimod also had significantly reduced metastatic melanoma in the lymph nodes, indicating that resiquimod can inhibit melanoma cell metastasis. Additionally, WO 2017/004421 showed that resiquimod in combination with systemic anti-PD1 therapy significantly enhanced the anti-PD1 therapeutic effects in melanoma models. Since the effect of resiquimod on tumors is mediated through activation of CD8 T cells and inhibition of myeloid-derived suppressor cells, the effect of resiquimod is not limited to melanocytic tumors. It is also effective in treating other epithelial cancers and cutaneous T-cell lymphoma.

However, also in view of the shear endless types of cancers and tumors there remains a need for further compounds to treat cancer and other diseases.

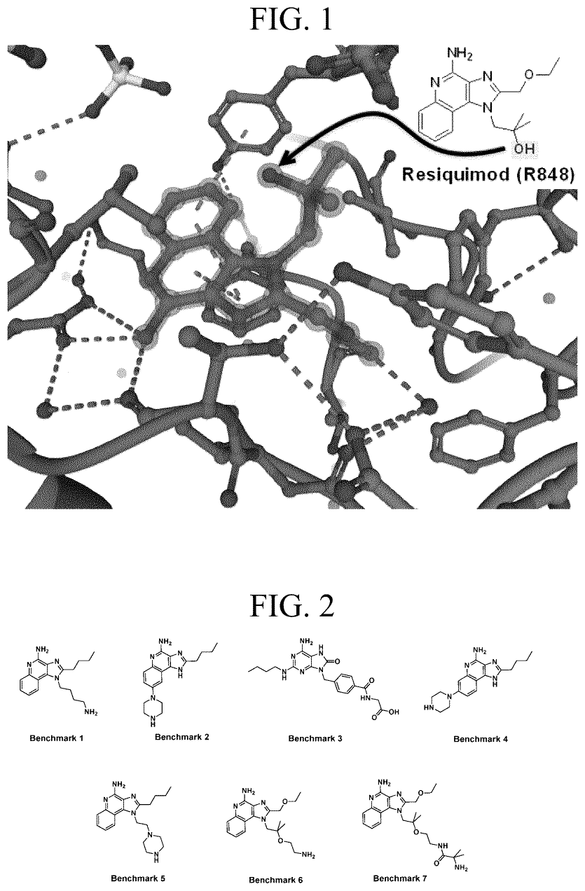

FIG. 1 shows a co-crystal structural analysis of TLR7 complexed with agonistic ligands like R848 (Shimizu et. al, Cell Reports 2018, 25, 3371). Unexpectedly, the inventors identified modification possibilities at the unburied hydrogen group of R848. The agonistic binding site in the ectodomain of TLR7 is located in the cellular lysosome. Thus, the inventors found that any modification with a protonatable group under a low pH value should have the potential to enhance the retention time of the agonistic ligand in the lysosome. The inventors reasoned that this will, in return, increase the binding between such agonistic ligands and TLR7 protein.

Accordingly, the inventors synthesized a large amount of different TLR7 agonists which are based on imidazoquinoline. From further experiments outlined in the example section below, it was—in alignment with the reasoning of the inventors—confirmed that the novel group of agonists performed very well in cell based experiments.

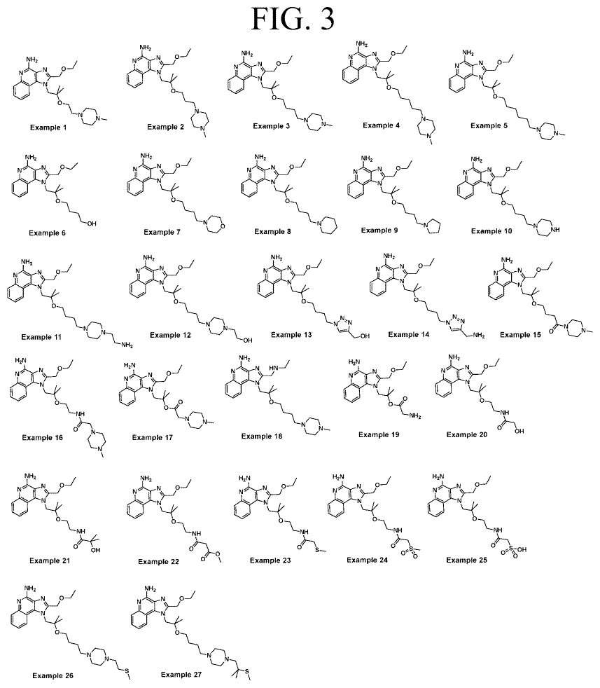

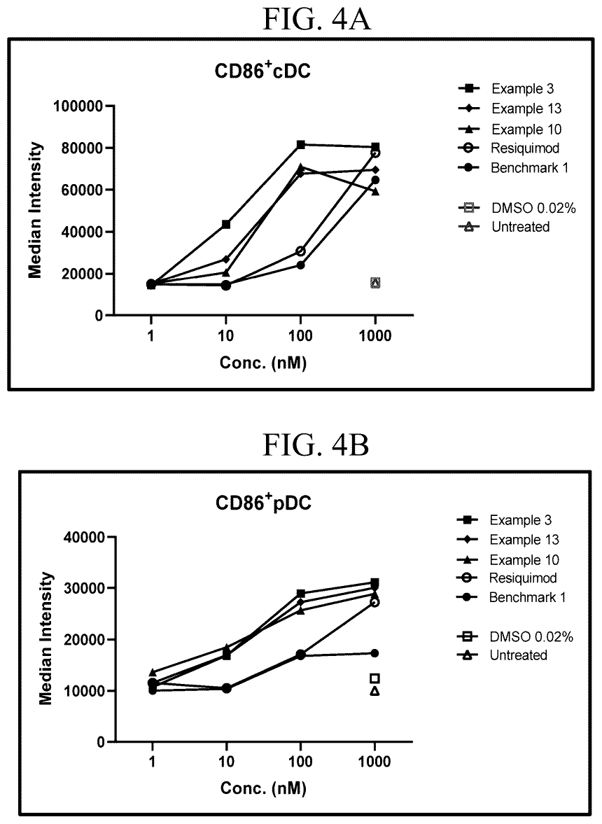

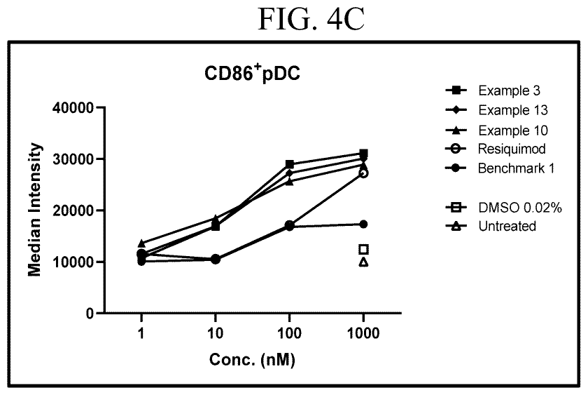

Unexpectedly, compared to R484 and some recently reported TLR7/8 agonists (FIG. 2), these novel TLR7 agonists (FIG. 3) not only showed very good activities in cellular TLR7 experiments, but also exhibited a high-selectivity between TLR7 and TLR8. More surprisingly, these novel high-selective TLR7 agonists initiated a high cytokine and chemokine (IL-6, IL1-b and TNF-a) production and induce strong upregulation of CD40 and CD86 in a cellular monocytes experiment (FIG. 4).

Accordingly, the invention provides in a first aspect a compound according to formula I

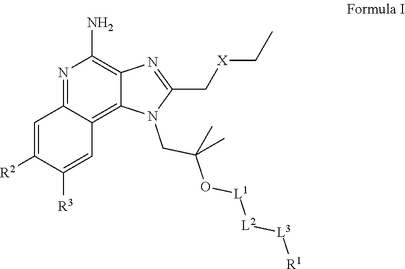

or a pharmaceutically acceptable salt thereof,
wherein X is an oxygen atom, C1-C5-alkyl (preferably CH₂) or NH;
wherein R² and R³ are each independently selected from the group consisting of hydrogen, C1-C5-alkyl, C4-C7-cycloalkyl, C4-C7-heterocycloalkyl, aryl and heteroaryl; preferably both R² and R³ are each hydrogen;
and wherein L¹, L², L³ and R¹ are as defined below under (a) or (b) or (c):
(a) L¹ is C2-C6 alkyl;
L² is a 5- or 6-membered heterocycle (preferably triazole or piperazine), or OH; L³ is absent or selected from the group consisting of hydrogen, C1-C2 alkyl and CH₂C(CH₃)₂; and
R1 is absent or selected from the group consisting of hydrogen, NH₂, OH and SCH₃;
(b) L¹ is C2-C6 alkyl;
L² is selected from the group consisting of C(O), NHC(O)CH₂ and NHC(O)C(CH₃)₂;
L³ is selected from the group consisting of a 6-membered heterocycle, OH, C(O)O, S, SO₂ and SO₃H; and
R1 is absent or methyl or hydrogen;
(c) L¹ is C(O)CH₂;
L² is a 6-membered heterocycle or NH₂;
L³ is not present; and
R¹ is methyl or hydrogen or absent;
and wherein if L³ is not present then L² is directly bound via a covalent bond to R¹.

In certain embodiments, compounds of the invention stimulate an immune response for treating cancer.

Before the aspects and embodiments of the invention are described in more detail certain definitions are provided below that apply to the invention disclosed herein throughout the entire specification including the claims.

### Definitions

Unless indicated or defined otherwise, all terms used have their usual meaning in the art, which will be clear to the skilled person. Reference is for example made to the standard handbooks, such as Sambrook et al. (Molecular Cloning: A Laboratory Manual (2^{nd} Ed.) Vols. 1-3, Cold Spring Harbor Laboratory Press, 1989), F. Ausubel et al. (Current protocols in molecular biology, Green Publishing and Wiley Interscience, New York, 1987), Lewin (Genes II, John Wiley & Sons, New York, N.Y., 1985), Old et al. (Principles of Gene Manipulation: An Introduction to Genetic Engineering (2nd edition) University of California Press, Berkeley, CA, 1981); Roitt et al. (Immunology (6th Ed.) Mosby/Elsevier, Edinburgh, 2001), Roitt et al. (Roitt's Essential Immunology (10^{th} Ed.) Blackwell Publishing, UK, 2001), and Janeway et al. (Immunobiology (6^{th} Ed.) Garland Science Publishing/Churchill Livingstone, New York, 2005), as well as to the general background art cited herein.

Unless indicated otherwise, all methods, steps, techniques and manipulations that are not specifically described in detail can be performed and have been performed in a manner known per se, as will be clear to the skilled person.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 15%, more preferably within 10%, and most preferably within 5% of a given value or range. Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

“Agonist” refers to a compound that can combine with a receptor (e.g., a TLR) to induce a cellular activity. An agonist may be a ligand that directly binds to the receptor. Alternatively, an agonist may combine with a receptor indirectly by, for example, (a) forming a complex with another molecule that directly binds to the receptor, or (b) otherwise results in the modification of another compound so that the other compound directly binds to the receptor. An agonist may be referred to as an agonist of a particular TLR (e.g., a TLR6 agonist) or a particular combination of TLRs (e.g., a TLR 7/8 agonist—an agonist of both TLR7 and TLR8). “Ameliorate” refers to any reduction in the extent, severity, frequency, and/or likelihood of a symptom or clinical sign characteristic of a particular condition.

“Cell-mediated immune activity” refers to a biological activity considered part of a cell-mediated immune response such as, for example, an increase in the production of at least one TH1 cytokine.

“Immune cell” refers to a cell of the immune system, i.e., a cell directly or indirectly involved in the generation or maintenance of an immune response, whether the immune response is innate, acquired, humoral, or cell-mediated.

“Sign” or “clinical sign” refers to an objective physical finding relating to a particular condition capable of being found by one other than the patient.

“Symptom” refers to any subjective evidence of disease or of a patient's condition.

“Treat” or variations thereof refer to reducing, limiting progression, ameliorating, or resolving, to any extent, the symptoms or signs related to a condition.

“Penetration enhancer” and “permeation enhancement” as used herein relates to an agent and the increase in the permeability of tissue to a drug respectively, i.e., so as to increase the rate and extent at which the drug permeates through a tissue such as skin or a tumor. The enhanced permeation effected through the use of such enhancers can be observed, for example, by measuring the rate of diffusion of drug through animal or human skin or tumor tissue using a diffusion cell apparatus or in situ measurements. The diffusion cell is described by Merritt et al. Diffusion Apparatus for Skin Penetration, J. of Controlled Release, 1 (1984) pp. 161-162.

“Adjuvancy” as used here relates to the ability to influence a non-specific inflammation or specific immune response caused by an activator of the immune system.

As used herein, the term “anti-cancer response” to therapy relates to any response of the cancer to therapy, preferably to a change in tumor mass and/or volume after initiation of therapy. Hyperproliferative disorder response may be assessed where the size of a tumor after topical or systemic intervention is compared to the initial size and dimensions as measured by CT, PET, mammogram, ultrasound or palpation. Response may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of therapy, e.g., after a few hours, days, weeks or preferably after a few months. A typical endpoint for response assessment is upon termination of chemotherapy or upon surgical removal of residual tumor cells and/or the tumor bed. This is typically three months after initiation of therapy.

As used herein “decreasing the size of a tumor” is defined as a reduction in the size of a tumor. Such an effect can be accomplished by reducing the number of proliferating tumor cells in the tumor (e.g., by reducing cell division of the tumor cells) and/or by inducing cytotoxicity or cell death (apoptosis) of existing tumor cells. Accordingly, tumor growth is arrested or prevented.

As used herein, the term “inhibiting cancer” or “inhibiting cancer cell growth” is intended to include the inhibition of undesirable or inappropriate cell growth. The inhibition is intended to include inhibition of proliferation including rapid proliferation. The term “inhibiting cancer cell growth” is also intended to encompass inhibiting tumor growth which includes the prevention of the growth of a tumor in a subject or a reduction in the growth of a pre-existing tumor in a subject. The inhibition also can be the inhibition of the metastasis of a tumor from one site to another. A cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

“Therapeutic compound”, “agent” and “therapeutic agent” are used herein synonymously.

As used herein, “a,” “an,” “the,” “at least one,” and “one or more” are used interchangeably. Thus, for example, a pharmaceutical composition comprising “an” immune response modulator (IRM) compound can be interpreted to mean that the pharmaceutical composition includes at least one IRM compound.

As used herein, the term “subject” shall mean any animal including, without limitation, a human, a mouse, a rat, a rabbit, a non-human primate, or any other mammal. In one embodiment, the subject is a primate. In another and most preferred embodiment, the subject is a human.

Also herein, the recitations of numerical ranges by endpoints include all numbers subsumed within that range (e.g., 1 to 5 includes 1, 1.5, 2, 2.75, 3, 3.80, 4, 5, etc.). The composition of the invention may for examples comprise a compound of the invention in an amount of 0.01%-1% (vol/wt %).

As used herein, the term “synergistic” refers to a combination of therapeutic agents described herein, which, when taken together, is more effective than the additive effects of the individual therapies. A synergistic effect of a combination of therapies (e.g., a combination of therapeutic agents) permits the use of lower dosages of one or more of the therapeutic agent(s) and/or less frequent administration of the agent(s) to a subject with a disease or disorder, e.g., a proliferative disorder. The ability to utilize lower dosages of one or more therapeutic agent and/or to administer the therapeutic agent less frequently reduces the toxicity associated with the administration of the agent to a subject without reducing the efficacy of the therapy in the treatment of a disease or disorder. In addition, a synergistic effect can result in improved efficacy of agents in the prevention, management or treatment of a disease or disorder, e.g. a proliferative disorder. Finally, a synergistic effect of a combination of therapies may avoid or reduce adverse or unwanted side effects associated with the use of either therapeutic agent alone.

As used herein, the term “in combination” may refer to the use of more than one therapeutic agent. The use of the term “in combination” does not restrict the order in which the therapeutic agents are administered to a subject with a disease or disorder, e.g., a proliferative disorder. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, such as an anti-cancer agent, to a subject with a disease or disorder, e.g. a proliferative disorder, such as cancer.

Immune response modifiers (“IRMs”) include compounds that possess potent immunomodulating activity including but not limited to antiviral and antitumor activity. Certain IRMs modulate the production and secretion of cytokines. For example, certain IRM compounds induce the production and secretion of cytokines such as, e.g., Type I interferons, TNF-alpha, IL-1, IL-6, IL-8, IL-0, IL-12, MUM, and/or MCP-1. As another example, certain IRM compounds can inhibit production and secretion of certain TH2 cytokines, such as IL-4 and IL-5.

Additionally, some IRM compounds are said to suppress IL-1 and TNF (U.S. Pat. No. 6,518,265). Resiquimod, (1-[4-amino-2-(ethoxymethyl) imidazo[4,5-c]quinolin-1-yl]-2-methylpropan-2-ol) is an immune response modifier (IRM) that works by stimulating cells through toll like receptors (TLR) 7 and 8.

As used herein, an “effective amount,” when used with respect to the compound of the invention and in respect of combination of agents described herein includes, without limitation, an amount of the agent or of each agent in the combination that provides a statistically significant desired effect on the disease or disorder that is to be treated (for example cancer). Representative desired effects are described herein. For example, in the context of cancer treatment the effect can be a decrease in the rate of tumor growth, a cessation of tumor growth, or a reduction in the size, mass, metabolic activity, or volume of the tumor, as measured by standard criteria such as, but not limited to, the Response Evaluation Criteria for Solid Tumors (RECIST), a statistically significant increase in survival relative to treatment with individual agents of the combination or sub-combinations of the combination alone, and the like. The effective amount can vary depending on such factors as the type of cell growth being treated or inhibited, the type of therapeutic agent(s) employed, the particular therapeutic agent, the size of the subject, or the severity of the cancer cell growth or tumor. For example, the choice of each of the individual agents which make up the combination can affect what constitutes an “effective amount”. One of ordinary skill in the art would be able to study the aforementioned factors and make the determination regarding the effective amount of the therapeutic compound or combination of therapeutic compounds/agents.

For example, an in vitro assay can be used to determine an “effective amount” of the therapeutic agents. The ordinarily skilled artisan would select an appropriate amount of each individual agent in the combination for use in the aforementioned in vitro assay. The cell survival fraction can be used to determine whether the selected amounts were an “effective amount” for the particular combination of therapeutic agents. For example, the selected amounts used within the assay preferably should result in a killing of at least 50% of the cells, more preferably 75%, and most preferably at least 95%. In a preferred embodiment, the effective dose of the therapeutic agent is a subtoxic dose. As used herein, the term subtoxic dose refers to a dose which results in the killing of less than about 10% of the cells.

The regimen (e.g., order) of administration can also affect what constitutes an effective amount. Further, several divided dosages, as well as staggered dosages, can be topically administered daily or sequentially, or the dose can be continuously infused. Further, the dosages can be proportionally increased or decreased as indicated by the exigencies of the therapeutic situation.

The phrase “pharmaceutically acceptable” is employed herein to refer to those therapeutic compounds of the invention or combinations of therapeutic compounds/agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

As used herein “alkyl” refers to a straight or branched chain hydrocarbon radical having a specified number of carbon atoms. Alkyl groups may be unsubstituted or substituted with substituents that do not interfere with the specified function of the composition and may be substituted once or twice with the same or different group. Substituents may include alkoxy, hydroxy, mercapto, amino, alkyl substituted amino, nitro, carboxy, carbonyl, carbonyloxy, cyano, methylsulfonylamino, or halogen, for example. Examples of “alkyl” include, but are not limited to, methyl, ethyl, n-propyl, isopropyl, n-butyl, s-butyl, t-butyl, n-pentyl, n-hexyl, 3-methylpentyl, and the like. A C1-C2 alkyl for example includes methyl and ethyl radicals. “Alkyl” hydrocarbons as used herein include mono- and bivalent-radicals, i.e. include alkylene radicals, if the resultant structure that comprises the alkyl group is stable.

The term “cycloalkyl,” as used herein, refers to a saturated monocyclic, bicyclic, or tricyclic hydrocarbon ring system having three to fourteen carbon atoms and zero heteroatoms. Representative examples of cycloalkyl groups include, but are not limited to, adamantyl, bicyclo[3.1.1]heptyl, cyclobutyl, cyclohexyl, cyclopentyl, and cyclopropyl.

The term “aryl” used alone or as part of a larger moiety as in “aralkyl”, “aralkoxy”, or “aryloxy alkyl”, refers to monocyclic and bicyclic ring systems having a total of five to fourteen ring members, wherein at least one ring in the system is aromatic and wherein each ring in the system contains three to seven ring members. The term “aryl” is used interchangeably with the term “aryl ring”. In certain embodiments of the present invention, “aryl” refers to an aromatic ring system. Exemplary aryl groups are phenyl, biphenyl, naphthyl, anthracyl and the like, which optionally includes one or more substituents. Preferably, an aryl group comprises no substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group is optionally mono- or bicyclic. The term “heteroaryl” is used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen is N (as in 3,4-dihydro-2H-pyrrolyl), NH (as in pyrrolidinyl), or +NR (as in N-substituted pyrrolidinyl).

A heterocyclic ring can be attached to its pendant group (or pendant groups) at any heteroatom or carbon atom that results in a stable structure and any of the ring atoms can be optionally substituted (non-substituted rings are preferred). Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group is optionally mono- or bicyclic. The term “heterocyclylalkyl” refers to an alkyl group substituted by a heterocyclyl, wherein the alkyl and heterocyclyl portions independently are optionally substituted (but are preferably non-substituted).

As described herein, certain compounds of the invention contain “optionally substituted” moieties. In general, the term “substituted”, whether preceded by the term “optionally” or not, means that one or more hydrogens of the designated moiety are replaced with a suitable substituent. When more than one position in any given structure is substituted with more than one substituent selected from a specified group, the substituent is either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein. A “stable” compound typically is not a radical. Preferably, compounds of the invention are not further substituted beyond those substitutions expressly indicated.

Unless otherwise indicated, reference to a compound and compounds of the invention can include the compound in any pharmaceutically acceptable form or pharmaceutically acceptable derivative, including any isomer (e.g., diastereomer or enantiomer), salt, ester, salt of an ester, solvate, polymorph, and the like. In particular, if a compound is optically active, reference to the compound can include each of the compound's enantiomers as well as racemic mixtures of the enantiomers. Additionally, unless otherwise stated, structures and compounds depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of this invention. In some embodiments, the group comprises one or more deuterium atoms. Preferably, a “pharmaceutically acceptable, salt, form or derivative” includes derivatives of a compound of this invention that, upon administration to a recipient, is capable of providing, either directly or indirectly, a compound of this invention or an agonistic metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. These salts can be prepared in situ during the final isolation and purification of the therapeutic agents, or by separately reacting a purified therapeutic agent in its free base form with a suitable organic or inorganic acid, and isolating the salt thus formed. Representative salts also include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19).

Salts derived from appropriate bases include alkali metal, alkaline earth metal, ammonium and N+(C1-4alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Therefore, in a first aspect the invention provides a compound according to formula I

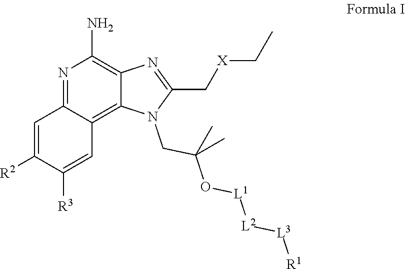

or a pharmaceutically acceptable salt or derivative thereof,
wherein X is an oxygen atom, C1-C5-alkyl (preferably CH₂) or NH;
wherein R² and R³ are each independently selected from the group consisting of hydrogen, C1-C5-alkyl, C4-C7-cycloalkyl, C4-C7-heterocycloalkyl, aryl and heteroaryl; preferably both
R² and R³ are each hydrogen;
and wherein L¹, L², L³ and R¹ are as defined below under (a) or (b) or (c):
(a) L¹ is C2-C6 alkyl;
L² is a 5- or 6-membered heterocycle or OH; L³ is absent or selected from the group consisting of hydrogen, C1-C2 alkyl and CH₂C(CH₃)₂; and
R¹ is absent or selected from the group consisting of hydrogen, NH₂, OH and SCH₃;
(b) L¹ is C2-C6 alkyl;
L² is selected from the group consisting of C(O), NHC(O)CH₂ and NHC(O)C(CH₃)₂;
L³ is selected from the group consisting of a 6-membered heterocycle, OH, C(O)O, S, SO₂ and SO₃H; and
R¹ is absent or methyl or hydrogen;
(c) L¹ is C(O)CH₂;
L² is a 6-membered heterocycle or NH₂;
L³ is not present; and
R¹ is methyl or hydrogen or absent;
and wherein if L³ is not present then L² is directly bound via a covalent bond to R¹.

In a preferred embodiment of the first aspect R² and R³ are hydrogen.

Preferably, X in the compound of the invention is an oxygen atom.

In a further embodiment, the compound of the invention has a structure according to formula II

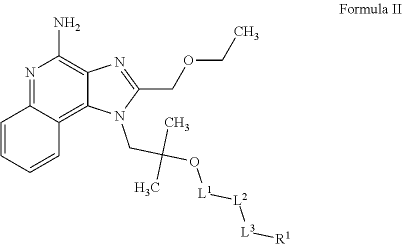

wherein L¹, L², L³ and R¹ are as defined below under (d), (e) or (f)
(d) L¹ is C2-C6 alkyl (e.g. ethyl, propyl, butyl, pentyl or hexyl);
L² is a 5- or 6-membered heterocycle (preferably triazole or piperazine), or OH; L³ is absent or selected from the group consisting of hydrogen, C1-C2 alkyl and CH₂C(CH₃)₂; and
R¹ is absent or selected from the group consisting of hydrogen, NH₂, OH and SCH₃;
(e) L¹ is C2-C3 alkyl;
L² is selected from the group consisting of C(O), NHC(O)CH₂ and NHC(O)C(CH₃)₂;
L³ is selected from the group consisting of a 6-membered heterocycle (preferably piperazine), OH, C(O)O, S, SO₂ and SO₃H; and
R¹ is absent or methyl or hydrogen;
(f) L¹ is C(O)CH₂;
L² is a 6-membered heterocycle (preferably piperazine) or NH₂;
L³ is not present; and
R1 is methyl or hydrogen or absent;
In more preferred embodiments of a compound with Formula I or II of the invention
L¹ is C2-C4 alkyl.

In a preferred embodiment of a compound with Formula II of the invention
L¹ is C2-C6 alkyl (preferably C2-C4 alkyl);
L² is a 5- or 6-membered heterocycle (preferably triazole or piperazine), or OH; L³ is absent or selected from the group consisting of hydrogen, C1-C2 alkyl and CH₂C(CH₃)₂; and
R¹ is absent or selected from the group consisting of NH₂, OH and SCH₃;

In a particularity preferred embodiment of the compound of the invention the compound has a structure selected from the group consisting of the structures listed below that are designated as Example 1 through Example 27:

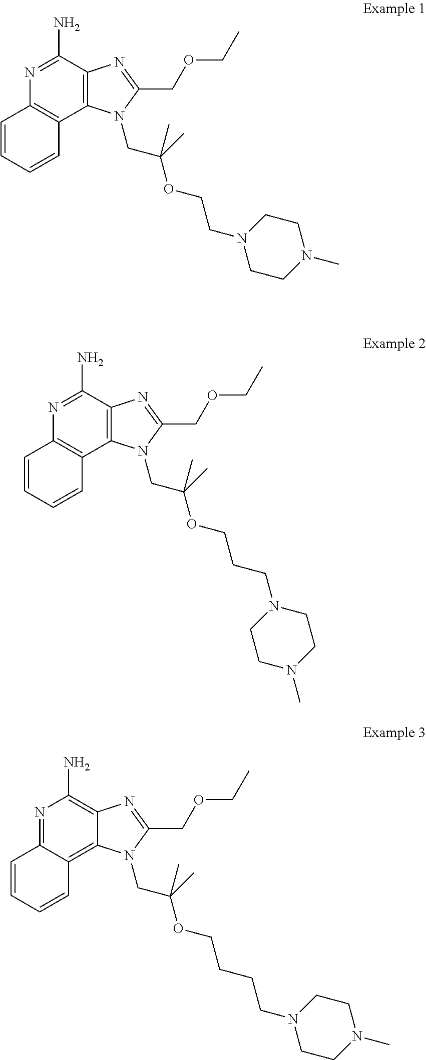 [SMILES: CCOCc1nc2c(N)nc3ccccc3c2n1CC(C)(C)OCCN1CCN(C)CC1 ; CCOCc1nc2c(N)nc3ccccc3c2n1CC(C)(C)OCCCN1CCN(C)CC1 ; CCOCc1nc2c(N)nc3ccccc3c2n1CC(C)(C)OCCCCN1CCN(C)CC1]

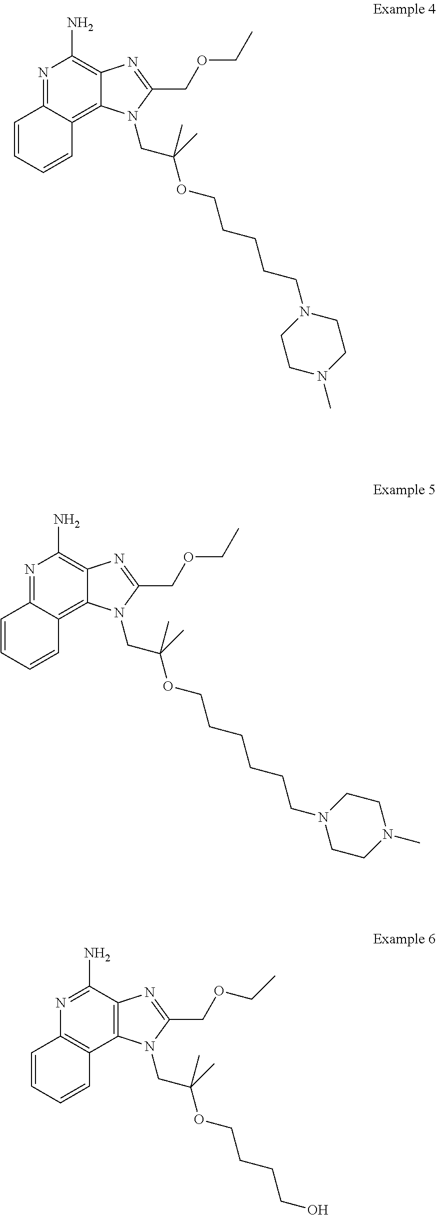 [SMILES: CCOCc1nc2c(N)nc3ccccc3c2n1CC(C)(C)OCCCCCN1CCN(C)CC1 ; CCOCc1nc2c(N)nc3ccccc3c2n1CC(C)(C)OCCCCCCN1CCN(C)CC1 ; CCOCc1nc2c(N)nc3ccccc3c2n1CC(C)(C)OCCCCO]

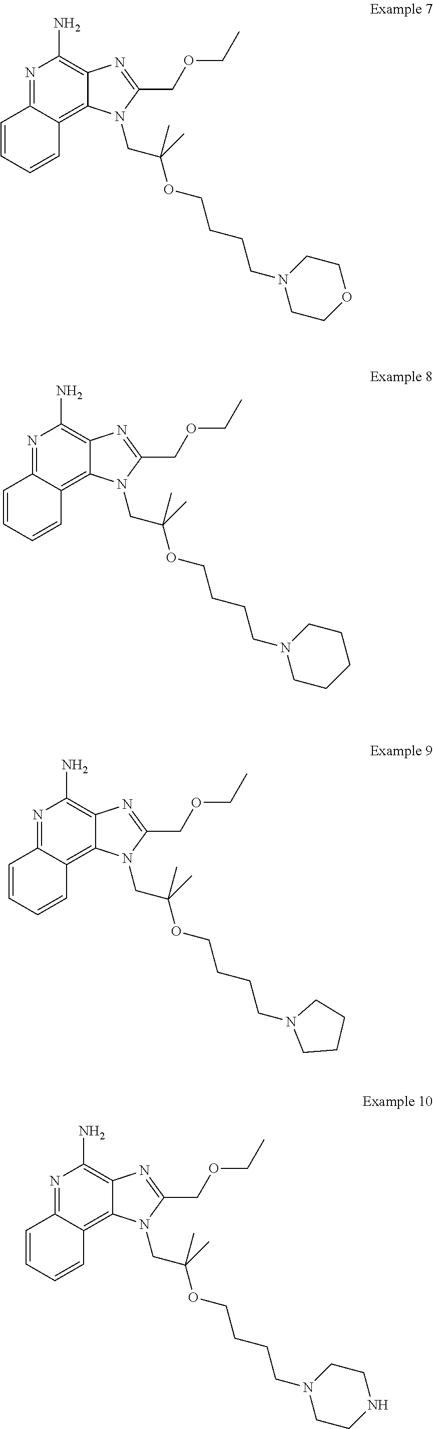 [SMILES: CCOCc1nc2c(N)nc3ccccc3c2n1CC(C)(C)OCCCCN1CCOCC1 ; CCOCc1nc2c(N)nc3ccccc3c2n1CC(C)(C)OCCCCN1CCCCC1 ; CCOCc1nc2c(N)nc3ccccc3c2n1CC(C)(C)OCCCCN1CCCC1 ; CCOCc1nc2c(N)nc3ccccc3c2n1CC(C)(C)OCCCCN1CCNCC1]

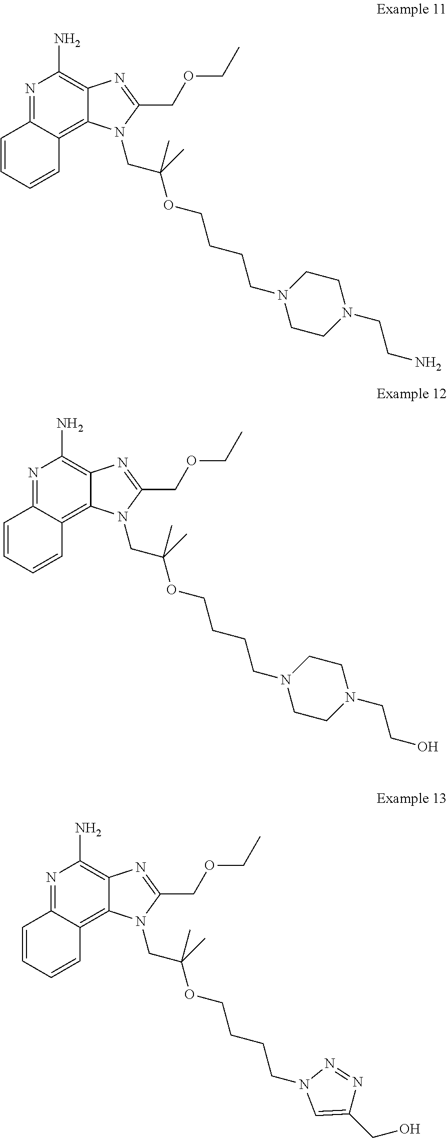 [SMILES: CCOCc1nc2c(N)nc3ccccc3c2n1CC(C)(C)OCCCCN1CCN(CCN)CC1 ; CCOCc1nc2c(N)nc3ccccc3c2n1CC(C)(C)OCCCCN1CCN(CCO)CC1 ; CCOCc1nc2c(N)nc3ccccc3c2n1CC(C)(C)OCCCCn1cc(CO)nn1]

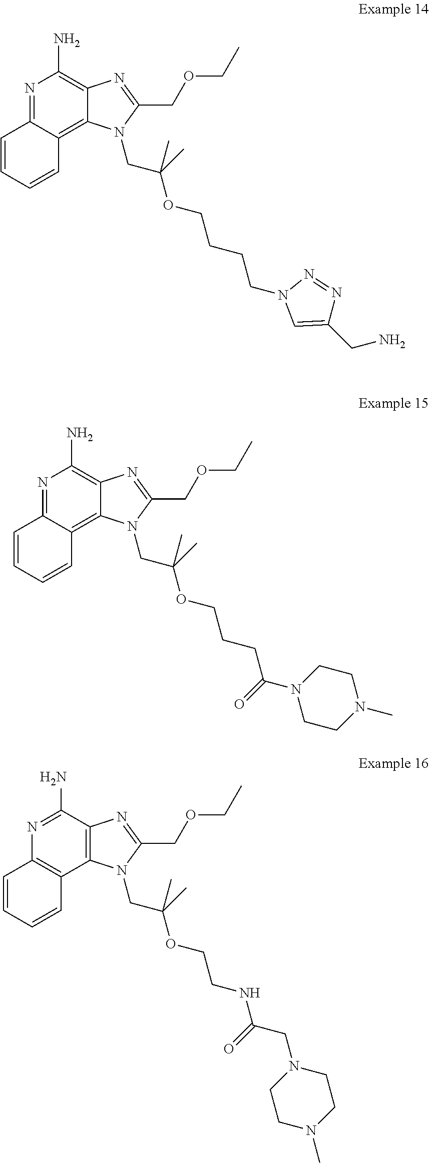 [SMILES: CCOCc1nc2c(N)nc3ccccc3c2n1CC(C)(C)OCCCCn1cc(CN)nn1 ; CCOCc1nc2c(N)nc3ccccc3c2n1CC(C)(C)OCCCC(=O)N1CCN(C)CC1 ; CCOCc1nc2c(N)nc3ccccc3c2n1CC(C)(C)OCCNC(=O)CN1CCN(C)CC1]

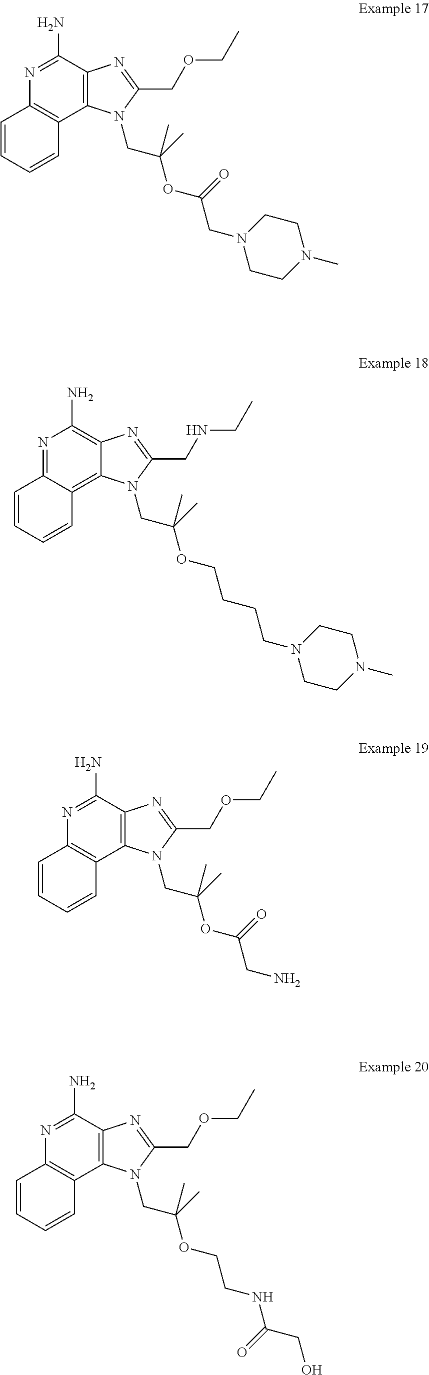 [SMILES: CCOCc1nc2c(N)nc3ccccc3c2n1CC(C)(C)OC(=O)CN1CCN(C)CC1 ; CCNCc1nc2c(N)nc3ccccc3c2n1CC(C)(C)OCCCCN1CCN(C)CC1 ; CCOCc1nc2c(N)nc3ccccc3c2n1CC(C)(C)OC(=O)CN ; CCOCc1nc2c(N)nc3ccccc3c2n1CC(C)(C)OCCNC(=O)CO]

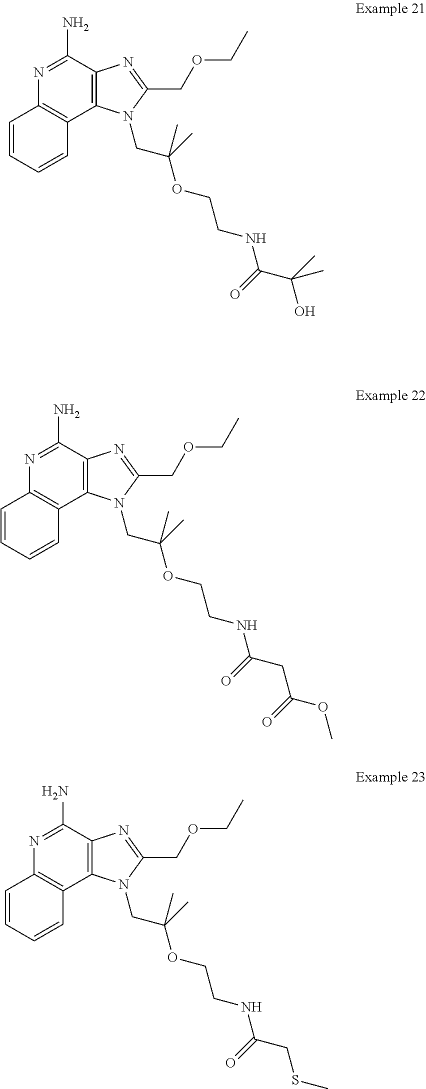 [SMILES: CCOCc1nc2c(N)nc3ccccc3c2n1CC(C)(C)OCCNC(=O)C(C)(C)O ; CCOCc1nc2c(N)nc3ccccc3c2n1CC(C)(C)OCCNC(=O)CC(=O)OC ; CCOCc1nc2c(N)nc3ccccc3c2n1CC(C)(C)OCCNC(=O)CSC]

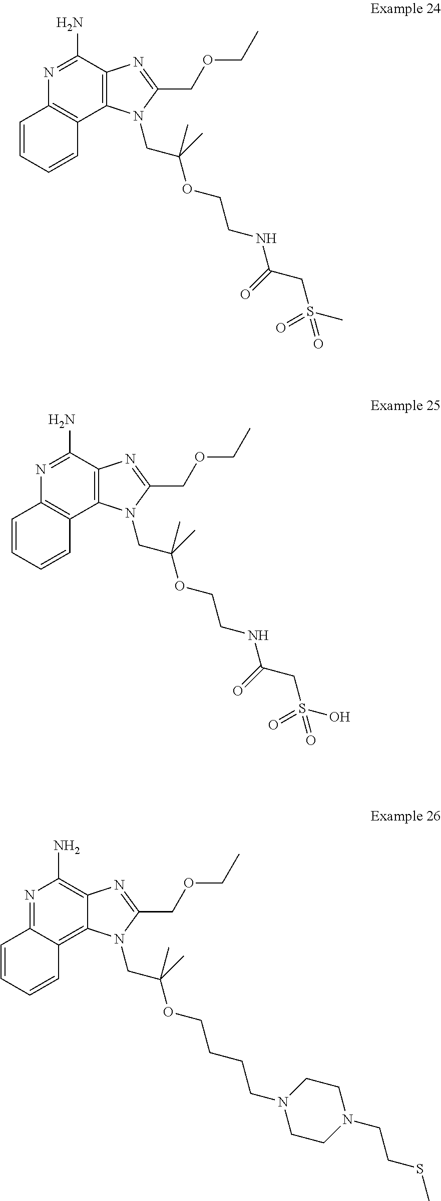 [SMILES: CCOCc1nc2c(N)nc3ccccc3c2n1CC(C)(C)OCCNC(=O)CS(C)(=O)=O ; CCOCc1nc2c(N)nc3ccccc3c2n1CC(C)(C)OCCNC(=O)CS(=O)(=O)O ; CCOCc1nc2c(N)nc3ccccc3c2n1CC(C)(C)OCCCCN1CCN(CCSC)CC1]

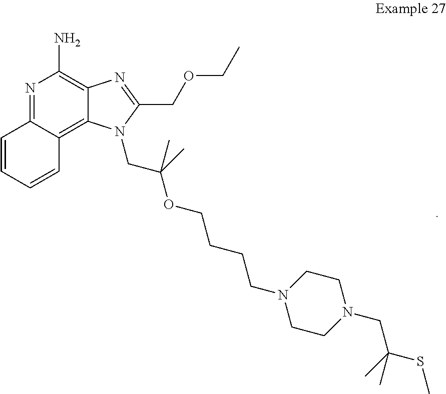 [SMILES: CCOCc1nc2c(N)nc3ccccc3c2n1CC(C)(C)OCCCCN1CCN(CC(C)(C)SC)CC1]

In preferred embodiments of the invention the compound of the invention is a compound according to Example 13 or 14 as outlined above, or according to Example 26 or 27 as outlined above.

In preferred embodiments the compound of the invention is a compound that is selected from the group consisting of compounds of Examples 1 through 6 as outlined above, or a pharmaceutically acceptable salt or derivative thereof. In most preferred embodiments the compound of the invention is a compound selected from the group consisting of Example 1, 3, 4 and 5 as outlined above, or a pharmaceutically acceptable salt or derivative thereof.

A further preferred compound of the invention relates to a compound wherein
X is NH;
L¹ is C2-C6 alkyl;
L² is a 5- or 6-membered heterocycle;
L³ is not present; and
R¹ is methyl or hydrogen; and
R² and R³ are each hydrogen.

Also preferred is a compound of the invention wherein the compound
(i) activates TLR7 more strongly than TLR8; and/or
(ii) induces IL-6, IL1-b and TNF-α production; and/or
(iii) induces upregulation of CD40 and/or CD86 in peripheral blood mononuclear cells (PBMC).

In the embodiment above, the average skilled person can test whether a compound activates TLR7 more strongly than TLR8 by carrying out the method provided below in Example 6.2.1. In the embodiment above, the average skilled person can test whether a compound induces TNF-α production by carrying out the method provided below in Example 6.2.2.

In the embodiment above, the average skilled person can test whether a compound induces upregulation of CD86 by carrying out the method provided below in Example 6.2.3.

Also preferred is a compound according to the invention, wherein the compound induces TNFα secretion from peripheral blood mononuclear cells (PBMC) if the compound contacts said cells. Also preferred is a compound according to the invention, wherein the compound has for TLR7 an EC50 of less than 10 μM and preferably less than 0.01 μM, preferably when tested in a test system described in Example 6.2.1 below.

A further aspect of the invention relates to a pharmaceutical composition comprising a compound of the invention.

The invention also relates to a pharmaceutical composition comprising the compound of the invention and preferably an additional compound such as imiquimod and/or resiquimod (R848). A further aspect of the invention relates to a compound of the invention for the use as a medicament.

A further aspect of the invention relates to a compound of the invention for the use in the treatment of a disease wherein said compound is used in said treatment in combination with the further compounds imiquimod and/or resiquimod (R848).

Preferred embodiments provide a compound of the invention for the use in the treatment of a condition selected from the group consisting of cancer, a viral infection, non-cancerous skin lesions, pre-cancerous skin lesions, cancerous skin lesions, bladder cancer, virally mediated skin disease, said composition optionally being formulated for enhanced penetration following topical administration, said composition preferably initiating a local specific inflammatory cytokine response while limiting undesirable erythema and other inflammatory reactions.

In preferred embodiments of the composition of the invention the compound of the invention is comprised in an amount of between 0.01%-1% wt/vol.

A further aspect relates to a composition of the invention for use in the treatment of bladder cancer, wherein the composition is formulated for intracystic administration.

In preferred embodiments of the composition of the invention the invention further comprises additional components of one of the groups i through xiii outlined below:
i. Oleic acid (50%) and isopropyl myristate (50%) in a solution containing 50 ml phosphate buffered saline and 50 ml ethanol at a total concentration of 1% wt/vol, or
ii. Isopropyl myristate (50%) and sodium lauryl sulfate (50%) in a solution containing 50 ml phosphate buffered saline and 50 ml ethanol at a total concentration of 0.5% wt/vol, or iii. Oleic acid (50%) and sodium lauryl sulfate (50%) in a solution containing 50 ml phosphate buffered saline and 50 ml ethanol at a total concentration of 0.5% wt/vol, or iv. Oleic acid (33%), isopropyl myristate (33%) and sodium lauryl sulfate (33%) in a solution containing 50 ml phosphate buffered saline and 50 ml ethanol at a total concentration of 0.5% wt/vol, or
v. Isopropyl palmitate (50%) and sodium oleate (50%) in a solution containing 50 ml phosphate buffered saline and 50 ml ethanol at a total concentration of 2% wt/vol, or
vi. Sodium lauryl sulfate (25%) and linoleic acid (75%) in a solution containing 50 ml phosphate buffered saline and 50 ml ethanol at a total concentration of 1.0% wt/vol, or vii. Palmitic acid (50%) and isopropyl laurate (50%) in a solution containing 50 ml phosphate buffered saline and 50 ml ethanol at a total concentration of 2.0% wt/vol, or
viii. Oleic acid (50%) and linoleic acid (50%) in a solution containing 50 ml phosphate buffered saline and 50 ml ethanol at a total concentration of 1.5% wt/vol, or
ix. Linoleic acid (25%), oleic acid (25%) and isopropyl linoleate (50%) in a solution containing 50 ml phosphate buffered saline and 50 ml ethanol at a total concentration of 0.5% wt/vol, or
x. Sodium oleate (33%), oleic acid (33%) and methyl palmitate (33%) in a solution containing 50 ml phosphate buffered saline and 50 ml ethanol at a total concentration of 2.0% wt/vol, or
xi. A solution containing 50 ml phosphate buffered saline and 50 ml ethanol, or
xii. Oleic acid (10%) in a solution containing 50 ml phosphate buffered saline and 50 ml ethanol, or
xiii. Oleic acid (2%) and sodium lauryl sulfate (5%) in a solution containing 50 ml phosphate buffered saline and 50 ml ethanol.

A further aspect of the invention relates to a method for the treatment of treatment of a cancer, comprising administering a therapeutically effective amount of the compound of the invention to the subject suffering from cancer.

Yet a further aspect of the invention provides a method for the treatment of skin tumor lesion and virus induced skin disease, comprising topical administration of the composition of the invention, said composition preferably being effective to reduce or eliminate said lesion or disease while limiting adverse skin reactions selected from erythema and inflammation, said composition preferably reducing tumor lesion penetration into surrounding tissue and metastasis to lymph nodes.

Yet a further aspect of the invention relates to a method for the treatment of bladder tumor, comprising intracystic administration of the composition of the invention, said composition preferably enhancing permeation and delivery of resiquimod to bladder epithelium while limiting irritation, said composition preferably reducing tumor invasion into surrounding muscular tissue and metastasis to lymph nodes.

In preferred embodiments of the method of the invention, said treatment further comprising systemic administration to said subject of at least one immune modulator selected from anti-PD1, anti-PD-L1, Anti-CTLA-4 antibody, anti-CD137 antibody, agonistic CD40 antibody, CD134 (Anti-OX40) agonist and PLX3397.

In preferred embodiments of the method of the invention, said treatment further comprising systemic administration to said subject of interferon gamma.

In preferred embodiments of the method of the invention, said treatment further comprising administration of local radiation with or without systemic anti-PD1 antibody.

In preferred embodiments of the method of the invention, said treatment further comprising of administration of photodynamic therapy.

In a further aspect the invention provides a method of activating TLR 7 and/or 8 in a biological sample, comprising contacting said biological sample with a compound according to the invention.

In a further aspect the invention provides a compound of the invention for use as a vaccine adjuvant or for use in the treatment of cancer in combination with an anti-cancer immunotherapy agent (preferably ipilimumab, nivolumab, or pembrolizumab).

### Preparation

The TLR7 agonist compounds of the invention can be prepared following the examples provided further below. Alternative methods of synthesizing the compounds of the invention can be also employed.

### Uses, Formulation and Administration

### Pharmaceutically Acceptable Compositions

Compounds of the invention will act as immune response modifiers, and will have antiviral and antitumour activity. For example, like resiquimod which is a toll-like receptor 7 (TLR7) agonist, it is expected that the compounds of the invention will, just as was shown for resiquimod in a clinical phase 2 study, reduce hepatitis C virus (HCV) infection. In a murine model of allergic asthma, resiquimod (i.n., 20 μg/mouse) reduces allergen induced airway reactivity and inflammation via reduction in Nrf2 signaling (Int J Biochem Cell Biol. 2016 April; 73:53-62. doi: 10.1016/j.biocel.2016.02.004. Epub 2016 Feb. 3). Resiquimod also modulates dendritic cells to augment cytomegalovirus- and HIV-1-specific T cell responses (J Immunol. 2003 Oct. 15; 171(8):4320-8. doi: 10.4049/jimmunol.171.8.4320). Resiquimod also induces the differentiation of myeloid-derived suppressor cells into macrophages and dendritic cells, and may improve cancer immunotherapy by reducing immunosuppressive MDSCs (see Arch Pharm Res. 2014; 37(9):1234-40. doi: 10.1007/s12272-014-0379-4. Epub 2014 Apr. 19.). In view of these multiple medical benefits that were shown for resiquimod as TLR7/8 agonist, it is expected that the compounds of the invention, which are likewise TLR7/8 agonists, will share the aforementioned medical uses and benefits.

As shown in the examples, the compounds of the invention are—just like resiquimod—also agonists for toll-like receptor 7 and 8. Accordingly, in a further aspect, the invention provides a composition comprising a compound of this invention or a pharmaceutically acceptable derivative thereof and a pharmaceutically acceptable carrier, adjuvant, or vehicle. In certain embodiments, the amount of a compound of the invention in compositions of this invention is such that is effective to measurably activate TLR7/8, or a mutant thereof, in a biological sample or in a patient. In certain embodiments, a composition of this invention is formulated for administration to a patient in need of such composition.

The term “patient” or “subject”, as used herein, means an animal, preferably a mammal, and most preferably a human.

The term “pharmaceutically acceptable carrier, adjuvant, or vehicle” includes nontoxic carriers, adjuvants, or vehicles that do not destroy the pharmacological activity of the compound with which it is formulated. Pharmaceutically acceptable carriers, adjuvants or vehicles that are used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention can be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention include aqueous or oleaginous suspension. These suspensions are formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that can be employed are water, Ringer's solution and isotonic sodium chloride solution. In addition, sterile, fixed oils can be conventionally employed as a solvent or suspending medium. A fixed oil that can be employed includes synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms can also be used for the purposes of formulation.

Pharmaceutically acceptable compositions of this invention can in one embodiment be orally administered in any orally acceptable dosage form. Exemplary oral dosage forms include capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient can be combined with emulsifying and suspending agents. If desired, certain sweetening, flavoring or coloring agents are optionally also added.

Alternatively, pharmaceutically acceptable compositions of this invention can be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches can also be used.

For topical applications, provided pharmaceutically acceptable compositions of the invention can be formulated in a suitable ointment containing the active compound of the invention suspended or dissolved in one or more carriers. Exemplary carriers for topical administration of compounds of this include mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions of this invention are optionally administered by nasal aerosol or inhalation. Such compositions can be prepared according to techniques well-known in the art of pharmaceutical formulation and are prepared as solutions in saline, employing benzyl alcohol or other suitable preservatives, absorption promoters to enhance bioavailability, fluorocarbons, and/or other conventional solubilizing or dispersing agents. As mentioned, pharmaceutically acceptable compositions of this invention can also be formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that are optionally combined with carrier materials to produce a composition in a single dosage form will vary depending upon the host treated, the particular mode of administration. Preferably, provided compositions can be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Immunocompromised patients are highly susceptible to development of severe infection which often progresses to the life-threatening condition of sepsis. Thus, immunotherapies aimed at boosting host immune defenses are highly attractive strategies to ward off infection and protect patients. Recently there has been mounting evidence that activation of the innate immune system can confer long-term functional reprogramming whereby innate leukocytes mount more robust responses upon secondary exposure to a pathogen for more efficient clearance and host protection, termed trained immunity. Toll-like receptor (TLR) agonists are a class of agents which have been shown to trigger the phenomenon of trained immunity through metabolic reprogramming and epigenetic modifications which drive profound augmentation of antimicrobial functions. Immunomodulatory TLR agonists are also highly beneficial as vaccine adjuvants. Furthermore, most cancer-associated antigens are self-antigens and require immunostimulant adjuvants in addition to cancer-targeting strategies. Thus, TLR7/8 agonists such as the compounds of the invention will also be useful in the treatment of cancer.

In one aspect, the invention provides a method for treating a subject suffering from a TLR7/8 related disorder, comprising administering to said subject an effective amount of a compound of the invention, preferably a compound of formula II and related formulae.

The compounds of the present invention are useful as anticancer agents for cancers that are responsive to TLR7 activation. In certain embodiments, the cancers include, but are not limited to cancer of the breast, bladder, bone, brain, central and peripheral nervous system, colon, endocrine glands, esophagus, endometrium, germ cells, head and neck, kidney, liver, lung, larynx and hypopharynx, mesothelioma, sarcoma, ovary, pancreas, prostate, rectum, renal, small intestine, soft tissue, testis, stomach, skin, ureter, vagina and vulva; inherited cancers, retinomblastoma and Wilms tumor; leukemia, lymphoma, non-Hodgkins disease, chronic and acute myeloid leukaemia, acute lymphoblastic leukemia, Hodgkins disease, multiple myeloma and T-cell lymphoma; myelodysplastic syndrome, plasma cell neoplasia, paraneoplastic syndromes, cancers of unknown primary site and AIDS related malignancies.

In certain embodiments, the compounds of the invention are used to treat cancers of the skin or kidney. Sensitivity of a given cancer to activation of TLR7 can be assessed by, but not limited to measurement of a decrease in primary or metastatic tumor load (minor, partial or complete regression), alterations in the hemogram, altered hormone or cytokine concentrations in the blood, inhibition of further increase of tumor load, stabilization of the disease in the patient, assessment of biomarkers or surrogate markers relevant for the disease, prolonged overall survival of a patient, prolonged time to disease progression of a patient, prolonged progression-free survival of a patient, prolonged disease-free survival of a patient, improved quality of life of a patient, or modulation of the co-morbidity of the disease (for example, but not limited to pain, cachexia, mobilization, hopitalization, altered hemogram, weight loss, wound healing, fever).

The compounds according to the present invention may further be useful as immune response modifiers that can modulate the immune response in a number of different ways, rendering them useful in the treatment of a variety of disorders.

Provided herein are methods of activating an immune response in an individual comprising administering to the individual an effective amount of an activator of TLR7 (e.g., TLR7 activator), using a compound as described herein. In some variations, the TLR activator activates a TLR7-dependent immune response. In some variations, the TLR activator activates a TLR7 and TLR8-dependent immune response. Unless otherwise noted, the term TLR activator refers to any one of the compounds of the invention disclosed herein. In some preferred embodiments, the individual is a human patient.

Methods of immunoregulation are provided by the present disclosure and include those that activate an immune response, including, but not limited to, an immune response. The present disclosure also provides methods for activating a TLR7 and/or TLR8 induced response (e.g., in vitro or in vivo). In some variations, the cell is contacted with the TLR activator in an amount effective to activate a response from the cell that contributes to an immune response.

Activation of TLR7 and/or TLR8 are useful for treating and/or preventing a variety of diseases or disorders that are responsive to cytokines.

Provided herein are methods of activating an immune response in an individual, the method comprising administering to the individual at least one TLR activator as disclosed herein in an amount effective to activate the immune response in the individual.

Provided herein are also methods of treating a viral disease or disorder e.g. an infection by HIV, the method comprising administering to the individual at least one compound of the invention in an amount effective to treat the viral disease or disorder.

In some embodiments of any of the methods involving administration of a compound of the invention to a subject the compound preferably has a therapeutically acceptable safety profile.

The compound/TLR activator may for example, have a therapeutically acceptable histological profile including an acceptably low, if any, toxicity of the liver, kidney, pancreas, or other organs. In some embodiments, a safety profile includes evaluation of toxicity, histological profile, and/or necrosis (e.g., liver, kidneys and/or heart). In some embodiments, the TLR activator has a therapeutically acceptable level of toxicity. In some embodiments, the TLR activator has a reduced level of toxicity as compared to another TLR activator. In some embodiments, the TLR activator induces a therapeutically acceptable reduction in body weight as compared to the initial body weight of a treated individual. In some embodiments, the TLR activator induces less than 5%, 7.5%, 10%, 12.5, or 15% reduction in total body weight. In some embodiments, the TLR activator has a therapeutically acceptable histology profile. In some embodiments, the TLR activator has a better (e.g., lower severity score) histology profile, for example, as compared to a reference TLR activator. In some embodiments, the TLR activator has a better (e.g., lower severity score) histology profile upon evaluation of the liver, kidneys and/or heart, for example. In some embodiments, the TLR activator has a therapeutically acceptable necrosis score. In some embodiments, the TLR activator has reduced necrosis and/or better (e.g., lower) necrosis score, for example, as compared to a reference TLR activator. In some embodiments, the TLR activator has reduced renal and/or hepatocellular necrosis and/or a better renal and/or hepatocellular necrosis score, for example, as compared to a reference TLR activator.

Accordingly, the invention provides a method of activating TLR7 in an animal, especially a mammal, preferably a human comprising administering an effective amount of a compound of the invention to the animal. An effective amount of a compound will vary according to factors known in the art but is expected to be a dose of about 0.1 to 10 mg/kg, 0.5 to 10 mg/kg, 1 to 10 mg/kg, 0.1 to 20 mg/kg, 0.1 to 20 mg/kg, or 1 to 20 mg/kg.

The invention also provides a method of treating a viral infection in an animal comprising administering an effective amount of a compound of the invention to the animal. An amount effective to treat or inhibit a viral infection is an amount that will cause a reduction in one or more of the manifestations of viral infection, such as viral lesions, viral load, rate of virus production, and mortality as compared to untreated control animals. The precise amount will vary according to factors known in the art but is expected to be a dose as indicated above with respect to the activation of TLR7, or a dose of about 100 ng/kg to about 50 mg/kg, preferably about 10 μg/kg to about 5 mg/kg.

The method of the invention can be performed either in-vitro or in-vivo. The susceptibility of a particular cell to treatment with the compounds according to the invention can be particularly determined by in-vitro tests, whether in the course of research or clinical application. Typically, a culture of the cell is combined with a compound according to the invention at various concentrations for a period of time which is sufficient to allow the active agents to inhibit TLR7/8 activity, usually between about one hour and one week. In-vitro treatment can be carried out using cultivated cells from a biopsy sample or cell line.

The host or patient can belong to any mammalian species, for example a primate species, particularly humans; rodents, including mice, rats and hamsters; rabbits; horses, cows, dogs, cats, etc. Animal models are of interest for experimental investigations, providing a model for treatment of human disease.

Moreover, the invention also provides the use of the compounds according to the invention and its derivatives for the production of a medicament for the prophylactic or therapeutic treatment of a disease that is caused, mediated and/or propagated by insufficient TLR7/8 activity. In certain embodiments, the invention provides the use of a compound according to the invention or physiologically acceptable salts or derivatives thereof, for the production of a medicament for the prophylactic or therapeutic treatment of a TLR7/8-mediated disorder. Compounds of the invention and/or a physiologically acceptable salt or derivate thereof can furthermore be employed as intermediate for the preparation of further medicament active ingredients. The medicament is preferably prepared in a non-chemical manner, e.g. by combining the active ingredient with at least one solid, fluid and/or semi-fluid carrier or excipient, and optionally in conjunction with a single or more other active substances in an appropriate dosage form.

The compounds of the invention can in one embodiment be administered before or following an onset of disease once or several times acting as therapy. The compounds are particularly used for the therapeutic treatment. A therapeutically relevant effect relieves to some extent one or more symptoms of a disorder, or returns to normality, either partially or completely, one or more physiological or biochemical parameters associated with or causative of a disease or pathological condition. Monitoring is considered as a kind of treatment provided that the compounds are administered in distinct intervals, e.g. in order to boost the response and eradicate the pathogens and/or symptoms of the disease completely. Either the identical compound or different compounds can be applied. The methods of the invention can also be used to reduce the likelihood of developing a disorder or even prevent the initiation of disorders associated with insufficient or reduced TLR7/8 activity (reduced compared to a healthy subject) in advance or to treat the arising and continuing symptoms.

In the meaning of the invention, prophylactic treatment is advisable if the subject possesses any preconditions for the aforementioned physiological or pathological conditions, such as a familial disposition, a genetic defect, or a previously incurred disease.

The invention furthermore relates in a further aspect to a medicament comprising at least one compound according to the invention and/or pharmaceutically usable derivatives, salts, solvates and stereoisomers thereof, including mixtures thereof in all ratios. In certain embodiments, the invention relates to a medicament comprising at least one compound according to the invention and/or physiologically acceptable salts thereof.

In various embodiments, the active ingredient may be administered alone or in combination with other treatments. A synergistic effect may be achieved by using more than one compound in the pharmaceutical composition, i.e. the compound of the invention is combined with at least another agent as active ingredient, which is either another compound of the invention or a compound of different structural scaffold. The active ingredients can be used either simultaneously or sequentially.

It was shown in the literature that the toll-like receptor 7 (TLR7) agonist, R848 in combination with radiation therapy (RT), leads to the longstanding clearance of tumor in T- and B-cell lymphoma bearing mice. Thus, in a further aspect the invention provides a method of treating a subject suffering from lymphoma by administering a compound of the invention in combination with treating the subject with radiation therapy.

This invention provides in a further aspect a method of treating cancer in a patient in need of such treatment comprising administering a therapeutically effective amount of a compound of the invention and administering a therapeutically effective amounts of at least one different antineoplastic agent selected from the group consisting of: (1) a taxane, (2) a platinum coordinator compound, (3) an epidermal growth factor (EGF) inhibitor that is an antibody, (4) an EGF inhibitor that is a small molecule compound, (5) a vascular endolithial growth factor (VEGF) inhibitor that is an antibody, (6) a VEGF kinase inhibitor that is a small molecule compound, (7) an estrogen receptor antagonist or a selective estrogen receptor modulator (SERMs), (8) an anti-tumor nucleoside derivative, (9) an epothilone, (10) a topoisomerase inhibitor, (11) a vinca alkaloid, (12) an antibody that is an inhibitors of αVβ3 integrins; (13) a small molecule compound that inhibits αVβ3 integrins; (14) a folate antagonist; (15) a ribonucleotide reductase inhibitor; (16) an anthracycline; (17) an anti-cancer biologic; (18) thalidomide (or related imid); and (19) Gleevec. Examples for these antineoplastic compounds are disclosed in US20060183765, incorporated herein by reference.

A compound of the invention can be administered in combination with further known therapeutic agents, including anticancer agents. As used here, the term “anticancer agent” relates to any agent which is administered to a patient with cancer for the purposes of treating the cancer.

The anti-cancer treatment may be applied as a monotherapy or may involve, in addition to the herein disclosed compounds of the invention, conventional surgery or radiotherapy or medicinal therapy. Such medicinal therapy, e.g. a chemotherapy or a targeted therapy, may include one or more, but preferably one, of the following anti-tumor agents:
Alkylating agents: such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, TH-302, VAL-083;
Platinum Compounds: such as carboplatin, cisplatin, eptaplatin, miriplatine hydrate, oxaliplatin, lobaplatin, nedaplatin, picoplatin, satraplatin; lobaplatin, nedaplatin, picoplatin, satraplatin;
DNA altering agents: such as amrubicin, bisantrene, decitabine, mitoxantrone, procarbazine, trabectedin, clofarabine; amsacrine, brostallicin, pixantrone, laromustine;
Topoisomerase Inhibitors: such as etoposide, irinotecan, razoxane, sobuzoxane, teniposide, topotecan; amonafide, belotecan, elliptinium acetate, voreloxin;
Microtubule modifiers: such as cabazitaxel, docetaxel, eribulin, ixabepilone, paclitaxel, vinblastine, vincristine, vinorelbine, vindesine, vinflunine; fosbretabulin, tesetaxel;
Antimetabolites: such as asparaginase, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur; doxifluridine, elacytarabine, raltitrexed, sapacitabine, tegafur, trimetrexate;
Anticancer antibiotics: such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin; aclarubicin, peplomycin, pirarubicin;
Hormones/Antagonists: such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol; acolbifene, danazol, deslorelin, epitiostanol, orteronel, enzalutamide;
Aromatase inhibitors: such as aminoglutethimide, anastrozole, exemestane, fadrozole, letrozole, testolactone; formestane;
Small molecule kinase inhibitors: such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib; afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifamib, tivantinib, tivozanib, trametinib, pimasertib, brivanib alaninate, cediranib, apatinib, cabozantinib S-malate, ibrutinib, icotinib, buparlisib, cipatinib, cobimetinib, idelalisib, fedratinib, XL-647;
Photosensitizers: such as methoxsalen; porfimer sodium, talaporfin, temoporfin;
Antibodies: such as alemtuzumab, besilesomab, brentuximab vedotin, cetuximab, denosumab, ipilimumab, ofatumumab, panitumumab, rituximab, tositumomab, trastuzumab, bevacizumab, pertuzumab; catumaxomab, elotuzumab, epratuzumab, farletuzumab, mogamulizumab, necitumumab, nimotuzumab, obinutuzumab, ocaratuzumab, oregovomab, ramucirumab, rilotumumab, siltuximab, tocilizumab, zalutumumab, zanolimumab, matuzumab, dalotuzumab, onartuzumab, racotumomab, tabalumab, abituzumab, nivolumab;
Cytokines: such as aldesleukin, interferon alfa, interferon alfa2a, interferon alfa2b; celmoleukin, tasonermin, teceleukin, oprelvekin, recombinant interferon beta-1a;
Drug Conjugates: such as denileukin diftitox, ibritumomab tiuxetan, iobenguane I123, prednimustine, trastuzumab emtansine, estramustine, gemtuzumab, ozogamicin, aflibercept; cintredekin besudotox, edotreotide, inotuzumab ozogamicin, naptumomab estafenatox, oportuzumab monatox, technetium (99mTc) arcitumomab, vintafolide;
Vaccines: such as sipuleucel; vitespe, emepepimut-S, oncoVAX, rindopepimut, tro Vax, MGN-1601, MGN-1703; and
Miscellaneous: alitretinoin, bexarotene, bortezomib, everolimus, ibandronic acid, imiquimod, lenalidomide, lentinan, metirosine, mifamurtide, pamidronic acid, pegaspargase, pentostatin, sipuleucel, sizofiran, tamibarotene, temsirolimus, thalidomide, tretinoin, vismodegib, zoledronic acid, vorinostat; celecoxib, cilengitide, entinostat, etanidazole, ganetespib, idronoxil, iniparib, ixazomib, lonidamine, nimorazole, panobinostat, peretinoin, plitidepsin, pomalidomide, procodazol, ridaforolimus, tasquinimod, telotristat, thymalfasin, tirapazamine, tosedostat, trabedersen, ubenimex, valspodar, gendicine4, picibanil4, reolysin4, retaspimycin hydrochloride1, trebananib, virulizin, carfilzomib, endostatin, immucothel, belinostat, MGN-1703.

Accordingly, aspects of the invention relate to the treatment of a subject by administering a compound of the invention together with one or more of the anti-tumor agnets listed above in suitable amounts to achieve a treatment of the cancer disease in said subject.

In some embodiments, the combination of a TLR inhibitor with one or more additional therapeutic agents reduces the effective amount (including, but not limited to, dosage volume, dosage concentration, and/or total drug dose administered) of the TLR activator and/or the one or more additional therapeutic agents administered to achieve the same result as compared to the effective amount administered when the TLR activator or the additional therapeutic agent is administered alone.

TLR activators also may be useful as a vaccine adjuvant for use in conjunction with any material that modulates either humoral and/or cell mediated immune response, such as, for example, live viral, bacterial, or parasitic immunogens; inactivated viral, tumor-derived, protozoal, organism-derived, fungal, or bacterial immunogens, toxoids, toxins; self-antigens; polysaccharides; proteins; glycoproteins; peptides; cellular vaccines; DNA vaccines; recombinant proteins; glycoproteins; peptides; and the like. In some aspects, the combination therapy of the invention includes but is not limited to the administration of a combination of a compound of the invention and a vaccine. In some aspects, the combination therapy of the invention includes but is not limited to the combination of a compound of the invention and a vaccine is used in the treatment of an infectious disease.

A further aspect of the invention relates to a kit comprising a compound of the invention as provided herein, and instructions for use in the methods of activating a TLR7- and/or TLR8-dependent immune response.

The kit may comprise one or more containers comprising a compound of the invention (or a formulation comprising said compound) as described herein, and a set of instructions, generally written instructions although electronic storage media (e.g., magnetic diskette or optical disk) containing instructions are also acceptable, relating to the use and dosage of the compound of the invention or formulation comprising said compound for the intended treatment (e.g., activation of TLR7 and/or TLR8, treating a virus infection, and/or treating and/or preventing one or more symptoms of a disease or disorder mediated by TLR7 and/or TLR8). The instructions included with the kit generally include information as to dosage, dosing schedule, and route of administration for the intended treatment. The containers for the TLR activator (or formulations comprising a TLR activator) may be unit doses, bulk packages (e.g., multi-dose packages) or sub-unit doses. The kits may further comprise a container comprising an adjuvant. In another aspect, the invention provides for a kit consisting of separate packs of an effective amount of a compound according to the invention and/or pharmaceutically acceptable salts, derivatives, solvates and stereoisomers thereof, including mixtures thereof in all ratios, and optionally, an effective amount of a further active ingredient. The kit comprises suitable containers, such as boxes, individual bottles, bags or ampoules. The kit may, for example, comprise separate ampoules, each containing an effective amount of a compound according to the invention and/or pharmaceutically acceptable salts, derivatives, solvates and stereoisomers thereof, including mixtures thereof in all ratios, and an effective amount of a further active ingredient in dissolved or lyophilized form.

Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention are administered orally or parenterally at dosage levels of about 0.01 mg/kg to about 100 mg/kg and preferably from about 1 mg/kg to about 50 mg/kg, of subject body weight per day, one or more times a day, to obtain the desired therapeutic effect. In certain embodiments, a therapeutically effective amount of a compound of the invention and related formulae and of the other active ingredient may depend on a number of factors, including, for example, the age and weight of the animal, the precise disease condition which requires treatment, and its severity, the nature of the formulation and the method of administration, and is ultimately determined by the treating doctor or vet. However, an effective amount of a compound is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as an individual dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound per se.

In certain embodiments, the pharmaceutical formulations can be administered in the form of dosage units, which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the disease condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process, which is generally known in the pharmaceutical art.

Liquid dosage forms for oral administration of a compound of the invention include, but are not limited to, pharmaceutically acceptable emulsions, microemulsions, solutions, suspensions, syrups and elixirs. In addition to the active compounds, the liquid dosage forms optionally contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions can be formulated according to the known art using suitable dispersing or wetting agents and suspending agents. The sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S. P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This can be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms can be made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration of a compound of the invention include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form also optionally comprises buffering agents.

Solid compositions of a similar type can also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type are also employed as fillers in soft and hard-filled gelatin capsules using such excipients as lactose or milk sugar as well as high molecular weight polethylene glycols and the like.

Compounds of the invention can also be formulated in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound of the invention may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms optionally also comprise buffering agents. They optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as required. Ophthalmic formulation, ear drops, and eye drops are also contemplated as being within the scope of this invention. Additionally, the present invention contemplates in preferred embodiments the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of activating TLR7/8 activity in a biological sample comprising the step of contacting said biological sample with a compound of this invention, or a composition comprising said compound. According to another embodiment, the invention relates to a method of activating TLR7/8, or a mutant thereof, activity in a biological sample, comprising the step of contacting said biological sample with a compound of this invention, or a composition comprising said compound.

The compounds of the invention are useful in-vitro as unique tools for understanding the biological role of TLR7/8, including the evaluation of the many factors thought to influence, and be influenced by, the production of TLR7/8 and the interaction of TLR7/8. The present compounds are also useful in the development of other compounds that interact with TLR7/8 since the present compounds provide important structure-activity relationship (SAR) information that facilitate that development. Compounds of the present invention that bind to TLR7/8 can preferably be used as reagents for detecting TLR7/8 in living cells, fixed cells, in biological fluids, in tissue homogenates, in purified, natural biological materials, etc. For example, by detectably labeling compounds of the invention, one can identify cells expressing TLR7/8. In addition, based on their ability to bind TLR7/8, compounds of the present invention can be used in in-situ staining, FACS (fluorescence-activated cell sorting), sodium dodecyl sulfate polyacrylamide gel electrophoresis ((SDS-PAGE), ELISA (enzyme-linked immunoadsorptive assay), etc., enzyme purification, or in purifying cells expressing TLR7/8 inside permeabilized cells. The compounds of the invention can also be utilized as commercial research reagents for various medical research and diagnostic uses. Such uses can include but are not limited to: use as a calibration standard for quantifying the activities of candidate TLR7/8 activators in a variety of functional assays; use as activating reagents in random compound screening, i.e. in looking for new families of TLR7/8 ligands; use in the co-crystallization with TLR7/8, i.e. the compounds of the present invention will allow formation of crystals of the compound bound to TLR7/8, enabling the determination of enzyme/compound structure by x-ray crystallography; other research and diagnostic applications, wherein TLR7/8 is preferably activated or such activation is conveniently calibrated against a known quantity of an TLR7/8 activator, etc.; use in assays as probes for determining the expression of TLR7/8 in cells; and developing assays for detecting compounds which bind to the same site as the TLR7/8 binding ligands. The compounds of the invention can be applied either themselves and/or in combination with physical measurements for diagnostics of treatment effectiveness. Pharmaceutical compositions containing said compounds and the use of said compounds to treat TLR7/8-mediated conditions is a promising, novel approach for a broad spectrum of therapies causing a direct and immediate improvement in the state of health, whether in human or in animal. The orally bioavailable and active new chemical entities of the invention improve convenience for patients and compliance for physicians.

The compounds of the invention, their salts, isomers, tautomers, enantiomeric forms, diastereomers, racemates, derivatives, prodrugs and/or metabolites are characterized by a high specificity and stability, low manufacturing costs and convenient handling. These features form the basis for a reproducible action, wherein the lack of cross-reactivity is included, and for a reliable and safe interaction with the target structure.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Modulation of TLR7/8, or a mutant thereof, activity in a biological sample is useful for a variety of purposes that are known to one of skill in the art. Examples of such purposes include, but are not limited to, blood transfusion, organ transplantation, biological specimen storage, and biological assays.

### Further Uses

In a further aspect, the present invention provides a pharmaceutical composition comprising a compound of the invention described herein and a pharmaceutically acceptable carrier, diluent, or excipient. In one embodiment, the pharmaceutical composition further comprises a therapeutically effective amount of a chemotherapeutic agent. In one aspect, the present invention provides a method for stimulating an immune response in a subject. The method includes administering a therapeutically effective amount of a compound of the invention described herein under conditions effective to stimulate an immune response. In some embodiments, the method is performed on a subject having cancer. In other embodiments, the cancer is selected from the group consisting of bladder cancer, breast cancer, cervical cancer, colon cancer, endometrial cancer, kidney cancer, lung cancer, esophageal cancer, ovarian cancer, prostate cancer, pancreatic cancer, skin cancer, gastric cancer, testicular cancer, biliary cancer, colorectal cancer, endometrial cancer, head/neck cancer, medullary thyroid cancer, renal cancer, eye cancer, neuroblastoma, Mycosis fungoides, glial tumor, other brain tumor, spinal cord tumor, liver cancer, leukemia, lymphoma, and any combination thereof. In certain embodiments, a compound of the invention can be comprised in a liquid pharmaceutical composition. The liquid composition of the invention can be administered into a tumor (e.g., intratumoral (IT) administration) to preferably induce an innate immune response and a cell-mediated immune response against the tumor antigens (e.g., shrink or stabilize the tumor). In a further embodiment, a compound of the invention is conjugated to a peptide. The conjugate comprising a peptide is not necessarily an antigen or immunogen, but a mechanism to reduce the solubility of the TLR7 and/or TLR8 agonist creating a depot that is retained at the site of administration, such as within a tumor or in the tumor microenvironment. A conjugated TLR7 and/or TLR8 agonist of the invention may stimulate immunosuppressive cells and may induce the immune response against the antigens present in the tumor. Moreover, mobilization of the immunosuppressive cells may induce an immune response against not only the tumor at the site of administration, but peripheral, nearby and/or distant tumors as well. In one embodiment, methods of stimulating an anti-tumor immune response in a subject are provided, where the methods comprise administering intratumorally or peritumorally a liquid form of the pharmaceutical composition of the invention into the subject, where the anti-tumor immune response will preferably be effective at a distant site from the site of administration of the pharmaceutical composition. The present invention also provides, in a further aspect, a method for inducing an anti-tumor immune response in a subject. The method includes administering a therapeutically effective amount of a compound of the invention described herein under conditions effective to induce an anti-tumor immune response. In some embodiments, the method is performed on a selected subject having a tumor. In some embodiments, the tumor is selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. The present invention provides, in a further aspect, a method for treating a tumor or abnormal cell proliferation in a subject. The method includes administering a therapeutically effective amount of a compound of the invention described herein under conditions effective to treat a tumor or abnormal cell proliferation. In some embodiments, the tumor or abnormal cell proliferation is cancer. In some embodiments, the cancer is selected from the group consisting of bladder cancer, breast cancer, cervical cancer, colon cancer, endometrial cancer, kidney cancer, lung cancer, esophageal cancer, ovarian cancer, prostate cancer, pancreatic cancer, skin cancer, gastric cancer, testicular cancer, biliary cancer, colorectal cancer, endometrial cancer, head and neck cancer, medullary thyroid cancer, renal cancer, eye cancer, neuroblastoma, Mycosis fungoides, glial tumor, other brain tumor, spinal cord tumor, liver cancer, leukemia, lymphoma, and any combination thereof. The present invention further provides, in yet another aspect, a method for treating an infectious disease in a subject. The method includes administering a therapeutically effective amount of a compound of the invention described herein under conditions effective to treat an infectious disease. In some embodiments, the infectious disease is a viral infection, a bacterial infection, a fungal infection, or any combination thereof. In some embodiments, the infectious disease is a viral infection, and the infectious disease is preferably selected from the group consisting of a coronavirus (including, but not limited to, Severe Acute Respiratory Syndrome (SARS), SARS-COV-2 (COVID-19), Middle East Respiratory Syndrome (MERS), and the common cold), Ebola, influenza, hepatitis, Hib disease, human immunodeficiency virus (HIV), human papillomavirus (HPV), meningococcal disease, pneumococcal disease, measles, mumps, norovirus, polio, respiratory syncytial virus (RSV), rotavirus, rubella virus, shingles, West Nile virus, rabies virus, enterovirus, cytomegalovirus, herpes virus, varicella, Yellow fever, Zika virus, and any combination thereof. In some embodiments, the infectious disease is a bacterial infection, and the infectious disease is preferably selected from the group consisting of streptococcal disease, staphylococcal disease, diphtheria, meningococcal disease, tetanus, pertussis, pneumococcal disease, bacterial food poisoning, a sexually transmitted infection, tuberculosis, Lyme disease, botulism, or any combination thereof. In some embodiments, the infectious disease is a fungal infection, and the infectious disease is candidiasis, histoplasmosis, dermatophytosis, tinea pedis, aspergillosis, cryptococcal meningitis, coccidioidomycosis, and any combination thereof.

### Topical, Intracystic and Intratumoral Compositions Comprising a Compound of the Invention for the Treatment of Diseases

A compound of the invention can be formulated together with other compounds such as a chemical penetration enhancer. Chemical penetration enhancers are often used in topical and transdermal formulations to enhance the absorption, uptake and delivery of active pharmaceutical ingredients (drug or drug substance) into the skin. Such formulations comprising a compound of the invention can be useful for example to treat skin cancer. Suitable formulations may also be employed to complement further permeation within a localized compartment of the tissue and/or to enhance an immune-related response in the tissue in which they are delivered. To enhance the permeation and uptake of the compound of the invention into the skin/tumor the formulation may comprise, but is not limited to, a combination of one or more chemical enhancers; one or more solvents or vehicles that improve portioning of the drug and chemical enhancers into the skin/tumor; and a gelling agent or matrix for their incorporation as a topical or intratumoral formulation. Several exemplars of chemicals under each category have been previously disclosed in the literature. However, the specific combination in which each ingredient is employed so that they match the physicochemical properties of the drug and improve its skin penetration are neither trivial nor singularly dependent on the specific properties of the individual chemicals. It is important to note that the effect of each ingredient on skin/tumor is dependent of the concentration employed in the formulation and there are synergistic effects to be anticipated from their combination in a single formulation. These effects can be additive, positively synergistic, or negatively synergistic. This then implies that the kinetics of skin/tumor penetration and cumulative effects of the drug delivered into and across the skin/tumor from these formulations depends on the specific formulation employed. It follows, therefore, that the physiological and biological and therapeutic endpoints of the drug is determined to a large extent by the formulation in addition to the drug properties. These endpoints include, but are not limited to, the pharmacokinetics/pharmacodynamics (PK/PD), cumulative absorption as determined by the area under the curve (AUC), bioequivalence, therapeutic index (TI), to highlight a few. Furthermore, the specific formulation of the drug affects not only its potency but also the tolerance/safety on the skin on application. This can include, but is not limited to, adverse effects such as irritation, skin toxicity, erythema amongst others.

A beneficial effect of chemical permeation enhancers in addition to enhancing the permeation of a compound of the invention across a tissue, or within a localized tissue compartment such as a tumor, is to provide an inflammatory effect that is influential in directing the immune response important for example in tumor therapy. Incorporation of agents in topical, intracystic and/or injectable formulations can therefore achieve additive or more than additive effects towards the therapeutic endpoint of tumor treatment.

Formulations which are suitable for administration include creams, ointments, solutions, gels, lotions, pastes, patches, foams or spray formulations containing such carriers as are known in the art to be appropriate. Dosage forms for the topical, intracystic or intratumoral administration of a compound of the invention and optionally other agents include powders, sprays, ointments, pastes, creams, ointments lotions, gels, solutions, and patches. The active component may be mixed under sterile conditions with a pharmaceutically-acceptable carrier, and with any preservatives, buffers, or propellants which may be required. The ointments, pastes, creams, lotions, solutions, foams and gels may contain, in addition to a therapeutic agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Injectable formulations comprising a compound of the invention may also be achieved by use of purified oils (soybean oil, safflower oil, triolein, and castor oil, fractionated coconut oil, miglyol810, 812, Neobee MS, Captex 300) and FDA approved dermal fillers (hydroxyl apatite, hyaluronic acid, Poly-L-Lactic Acid, collagen, hydrogel) as components of injectable formulation. Topical, intracystic and intratumoral formulations comprising a compound of the invention can be designed that are optimized by selecting the specific ingredients of the formulation including the enhancer, vehicle and the matrix (Karande 2004 Nature Biotechnology, Karande 2005 PNAS, and related references within and citing these works). Specific formulation can have a profound effect on efficacy of the drug and the target indication. The physicochemical properties of an active compound can be derived from its structure and chemical composition. Chemical descriptors for permeation enhancers can be estimated that allow to balance their potency and safety. Based on this combinations of chemical permeation enhancers can be identified that contain chemicals from the list below in the total concentration range of 0-2% wt/vol in weight fractions ranging between 0 and 100%. The formulation may contain 2 or 3 individual chemicals in a solvent to make the formulation. A compound of the invention can be combined in a formulation comprising one or more of the following additives: Sodium octyl sulfate, Sodium decyl sulfate, Sodium dodecyl sulfate, Sodium tetradecyl sulfate, Sodium heptadecyl sulfate, Sodium eicosyl sulfate, Sodium laureth sulfate, Nicotine sulfate, Sodium taurocholic sulfate, Dimethyl sulfoxide, Sodium tridecyl phosphate, ChemBetaine CAS, ChemBetaine Oleyl, ChemBetaine C, Hexadecyldimethyl ammonio propane sulfonate, Decyldimethyl ammonio propane sulfonate, Dodecyldimethyl ammonio propane sulfonate, Myristyldimethyl ammonio propane sulfonate, Benzyl pyridinium chloride, Dodecyl pyridinium chloride, Cetyl pyridinium chloride, Benzyldimethyl dodecyl ammonium chloride, Benzyldimethyl myristyl ammonium chloride, Benzyldimethyl stearyl ammonium chloride Octyltrimethyl ammonium bromide, Decyltrimethyl ammonium bromide, Dodecyltrimethyl ammonium bromide, Myristyltrimethyl ammonium chloride, Cetyltrimethyl ammonium bromide Sorbitan monolaurate, Sorbitan monopalmitate, Sorbitan monostearate, Sorbitan monooleate Polyoxyethylene sorbitan monolaurate, Polyoxyethylene sorbitan monopalmitate, Brij 97 Brij 30, Brij 56, Triton X-100, Hexanoic acid, Octanoic acid, Decanoic acid, Undecanoic acid Dodecanoic acid, Tridecanoic acid, Myristic acid, Palmitic acid, Stearic acid, Oleic acid, Elaidic acid, Linoleic acid, Linolenic acid, Cholic acid, Methyl hexanoate, Ethyl undecanoate, Methyl laurate, Methyl tridecanoate, Methyl myristate, Isopropyl myristate, Isopropyl palmitate, Palmityl palmitate, Diethyl sebaccate, Tetracaine, Glyceryl monolaurate, Glyceryl monooleate Ethylpiperazine carboxylate, N-Lauryl sarcosinate, Sodium caprylate, Sodium decanoate, Sodium palmitate, Sodium oleate, Octyl amine, Decyl amine, Dodecyl amine, Tetradecyl amine, Oleyl amine, Urea, Methyl pyrrolidone, Cyclohexyl pyrrolidone, Octyl pyrrolidone, Decyl pyrrolidone, Decyl methyl pyrrolidone, Methyl piperazine, Phenyl piperazine, Octanamide Hexadecanamide, Caprolactam, Carveol, Pinene oxide, Limonene, Menthol, Pulegone, Carvacrol Pinene, Menthone, Terpineol, Cineole, Fenchone, Triacetin, Trimethoxy propylene methyl benzene, Linalool, Geraniol, and Octyl dodecanol. Others include sulfoxides, alcohols, polyols, alkanes, fatty acids, esters, amines and amides, terpenes, surface-active agents, cyclodextrins, C2 or C3 alcohols and higher, a C3 or C4 diol or higher, DMSO, DMF, DMA and related solvents, 1-n-dodecyl-cyclazacycloheptan-2-one, N-methyl-pyrrolidone and N-(2-hydroxyethyl) pyrrolidone, and a broader class of azones, and mixtures (binary, ternary or higher)

A compound of the invention can be formulated with chemical enhancers that can improve transport of the compound. The optional chemical enhancers include long chain hydrocarbons with a polar head group such as surfactants, fatty acids and fatty esters.

Unsaturation (single or double) in the hydrocarbon chain can additionally aid the fluidization of skin lipid bilayers and permeation of a compound of the invention. Oleic acid, linoleic acid, linolinic acid, palmitic acid, myristic acid, etc. are exemplars of fatty acids. Esters of fatty acids such as palmitate, myristate, oleate, linoleate, etc. with a small alkyl group such as methyl, ethyl, propy or butyl are examples of fatty esters. Salts of fatty esters such as sodium lauryl sufate, sodium oleate, etc, are examples of surfactants. Short chain alcohols including ethanol, isopropanol, butanol, hexanol, etc. are examples of solvents. Combinations of chemicals and solvent can significantly enhance the permeation of a compound of the invention across the skin if the compound of the invention is formulated as topical formulation. Combinations of such exemplars are viable formulations with varying potency and safety with varying PK/PD profiles for the API which is a compound of the invention.

Examples of formulations that embody the chemicals noted above and their combinations are as follows: A compound of the invention in a concentration of 0.01, 0.02, 0.03, 0.04 to 1 wt/vol %, wherein the compound is preferably combined with one or more and preferably all of the components listed in i, ii, iii, iv, v, vi, vii, viii, ix, x, xi, xii or xiii below:
i. Oleic acid (50%) and isopropyl myristate (50%) in a solution containing 50 ml phosphate buffered saline and 50 ml ethanol at a total concentration of 1% wt/vol.
ii. Isopropyl myristate (50%) and sodium lauryl sulfate (50%) in a solution containing 50 ml phosphate buffered saline and 50 ml ethanol at a total concentration of 0.5% wt/vol.
iii. Oleic acid (50%) and sodium lauryl sulfate (50%) in a solution containing 50 ml phosphate buffered saline and 50 ml ethanol at a total concentration of 0.5% wt/vol.
iv. Oleic acid (33%), isopropyl myristate (33%) and sodium lauryl sulfate (33%) in a solution containing 50 ml phosphate buffered saline and 50 ml ethanol at a total concentration of 0.5% wt/vol.
v. Isopropyl palmitate (50%) and sodium oleate (50%) in a solution containing 50 ml phosphate buffered saline and 50 ml ethanol at a total concentration of 2% wt/vol.
vi. Sodium lauryl sulfate (25%) and linoleic acid (75%) in a solution containing 50 ml phosphate buffered saline and 50 ml ethanol at a total concentration of 1.0% wt/vol.
vii. Palmitic acid (50%) and isopropyl laurate (50%) in a solution containing 50 ml phosphate buffered saline and 50 ml ethanol at a total concentration of 2.0% wt/vol.
viii. Oleic acid (50%) and linoleic acid (50%) in a solution containing 50 ml phosphate buffered saline and 50 ml ethanol at a total concentration of 1.5% wt/vol.
ix. Linoleic acid (25%), oleic acid (25%) and isopropyl linoleate (50%) in a solution containing 50 ml phosphate buffered saline and 50 ml ethanol at a total concentration of 0.5% wt/vol.
x. Sodium oleate (33%), oleic acid (33%) and methyl palmitate (33%) in a solution containing 50 ml phosphate buffered saline and 50 ml ethanol at a total concentration of 2.0% wt/vol.
xi. A solution containing 50 ml phosphate buffered saline and 50 ml ethanol.
xii. Oleic acid (10%) in a solution containing 50 ml phosphate buffered saline and 50 ml ethanol.
xiii. Oleic acid (2%) and sodium lauryl sulfate (5%) in a solution containing 50 ml phosphate buffered saline and 50 ml ethanol.

### Medicaments and Further Uses of the Compounds and Compositions of the Invention

As mentioned aspect of the present invention include the provision of a compositions according to the invention for use as a medicament.

The dosage regimen will be determined by the attending physician and clinical factors. As is well known in the medical arts, dosage for any one patient depends upon many factors, including the patient's size, weight, body surface area, age, the particular compound to be administered, the activity of the employed compound, time and route of administration, general health, and combination with other therapies or treatments. Proteinaceous pharmaceutically active matter may be present in amounts between 1 g and 100 mg/kg body weight per dose; however, doses below or above this exemplary range are also envisioned. If the regimen is a continuous infusion, it may be in the range of 1 μg to 100 mg per kilogram of body weight per minute.

The specific amount(s) or doses to be administered can be determined by the clinician, again based on the factors cited above. Useful dosages of the compounds of the invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, depending on the specific disease, disorder or condition to be treated, the potency of the specific compound of the invention, the specific route of administration and the specific pharmaceutical formulation or composition used, the clinician will be able to determine a suitable dose.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. Preferably, the dose is administered once per week or even less frequent, such as once per two weeks, once per three weeks, once per month or even once per two months.

An administration regimen could include long-term treatment. By “long-term” is meant at least two weeks and preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed. 4), Mack Publishing Co., Easton, PA. The dosage can also be adjusted by the individual physician in the event of any complication.

Usually, in the above method, a compound of the invention will be used. It is however within the scope of the invention to use two or more different compounds of the invention in combination or a compound of the invention in combination with a further medicament.

Again, the clinician will be able to select such further compounds or medicament, as well as a suitable combined treatment regimen, based on the factors cited above and his expert judgment.

When two or more substances or principles are to be used as part of a combined treatment regimen, they can be administered via the same route of administration or via different routes of administration, at essentially the same time or at different times (e.g. essentially simultaneously, consecutively, or according to an alternating regime). When the substances or principles are to be administered simultaneously via the same route of administration, they may be administered as different pharmaceutical formulations or compositions or part of a combined pharmaceutical formulation or composition, as will be clear to the skilled person.

Also, when two or more active substances or principles are to be used as part of a combined treatment regimen, each of the substances or principles may be administered in the same amount and according to the same regimen as used when the compound or principle is used on its own, and such combined use may or may not lead to a synergistic effect. However, when the combined use of the two or more active substances or principles leads to a synergistic effect, it may also be possible to reduce the amount of one, more or all of the substances or principles to be administered, while still achieving the desired therapeutic action. This may for example be useful for avoiding, limiting or reducing any unwanted side-effects that are associated with the use of one or more of the substances or principles when they are used in their usual amounts, while still obtaining the desired pharmaceutical or therapeutic effect.

The effectiveness of the treatment regimen used according to the disclosure may be determined and/or followed in any manner known per se for the disease, disorder or condition involved, as will be clear to the clinician. The clinician will also be able, where appropriate and on a case-by-case basis, to change or modify a particular treatment regimen, so as to achieve the desired therapeutic effect, to avoid, limit or reduce unwanted side-effects, and/or to achieve an appropriate balance between achieving the desired therapeutic effect on the one hand and avoiding, limiting or reducing undesired side effects on the other hand.

Generally, the treatment regimen will be followed until the desired therapeutic effect is achieved and/or for as long as the desired therapeutic effect is to be maintained. Again, this can be determined by the clinician.

Thus, in a further aspect, the invention relates to a pharmaceutical composition that contains at least one compound of the invention and at least one suitable carrier, diluent or excipient (i.e., suitable for pharmaceutical use), and optionally one or more further active substances. In a particular aspect, the invention relates to a pharmaceutical composition that comprises a compound according to the invention (preferably at least one compound selected from examples 1 through 27 shown in the claims and more preferably a compound selected from example 1 through 6 of the list shown in the claims) and at least one suitable carrier, diluent or excipient (i.e., suitable for pharmaceutical use), and optionally one or more further active substances.

The subject to be treated may be any warm-blooded animal, but is in particular a mammal, and more in particular a human being. In veterinary applications, the subject to be treated includes any animal raised for commercial purposes or kept as a pet. As will be clear to the skilled person, the subject to be treated will in particular be a person suffering from, or at risk of, the diseases, disorders and conditions mentioned herein. Hence, in a preferred embodiment of the invention, the pharmaceutical compositions comprising a compound of the invention are for use in medicine or diagnostics. Preferably, the pharmaceutical compositions are for use in human medicine, but they may also be used for veterinary purposes.

Again, in such a pharmaceutical composition, the one or more compounds of the invention may also be suitably combined with one or more other active principles, such as those mentioned herein.

The invention also relates to a pharmaceutical composition for use, either in vitro (e.g. in an in vitro or cellular assay) or in vivo (e.g. in a single cell or multi-cellular organism, and in particular in a mammal, and more in particular in a human being, such as in a human being that is at risk of or suffers from a disease, disorder or condition of the invention).

It is to be understood that reference to treatment includes both treatment of established symptoms and prophylactic treatment, unless explicitly stated otherwise.

Generally, for pharmaceutical use, the compounds of the invention may be formulated as a pharmaceutical preparation or composition comprising at least one compound used in the invention and at least one pharmaceutically acceptable carrier, diluent or excipient and/or adjuvant, and optionally one or more pharmaceutically active polypeptides and/or compounds. By means of non-limiting examples, such a formulation may be in a form suitable for oral administration, for parenteral administration (such as by intravenous, intramuscular or subcutaneous injection or intravenous infusion), for topical administration (such as intra-articular administration), for administration by inhalation, by a skin patch, by an implant, by a suppository, etc., wherein the intra-articular administration is preferred. Such suitable administration forms—which may be solid, semi-solid or liquid, depending on the manner of administration—as well as methods and carriers for use in the preparation thereof, will be clear to the skilled person, and are further described herein. Such a pharmaceutical preparation or composition will generally be referred to herein as a “pharmaceutical composition”.

As exemplary excipients, disintegrators, binders, fillers, and lubricants may be mentioned. Examples of disintegrators include agar-agar, algins, calcium carbonate, cellulose, colloid silicon dioxide, gums, magnesium aluminium silicate, methylcellulose, and starch. Examples of binders include micro-crystalline cellulose, hydroxymethyl cellulose, hydroxypropylcellulose, and polyvinylpyrrolidone. Examples of fillers include calcium carbonate, calcium phosphate, tribasic calcium sulfate, calcium carboxymethylcellulose, cellulose, dextrin, dextrose, fructose, lactitol, lactose, magnesium carbonate, magnesium oxide, maltitol, maltodextrins, maltose, sorbitol, starch, sucrose, sugar, and xylitol. Examples of lubricants include agar, ethyl oleate, ethyl laureate, glycerin, glyceryl palmitostearate, hydrogenated vegetable oil, magnesium oxide, stearates, mannitol, poloxamer, glycols, sodium benzoate, sodium lauryl sulfate, sodium stearyl, sorbitol, and talc. Usual stabilizers, preservatives, wetting and emulsifying agents, consistency-improving agents, flavour-improving agents, salts for varying the osmotic pressure, buffer substances, solubilizers, diluents, emollients, colorants and masking agents and antioxidants come into consideration as pharmaceutical adjuvants.

Suitable carriers include but are not limited to magnesium carbonate, magnesium stearate, talc, sugar, lactose, pectin, dextrin, starch, gelatine, tragacanth, methylcellulose, sodium carboxymethyl-cellulose, a low melting-point wax, cocoa butter, water, alcohols, polyols, glycerol, vegetable oils and the like.

Generally, the compounds of the invention can be formulated and administered in any suitable manner known per se. Reference is for example made to the general background art cited above (and in particular to WO 04/041862, WO 04/041863, WO 04/041865, WO 04/041867 and WO 08/020079) as well as to the standard handbooks, such as Remington's Pharmaceutical Sciences, 18^{th} Ed., Mack Publishing Company, USA (1990), Remington, the Science and Practice of Pharmacy, 21st Edition, Lippincott Williams and Wilkins (2005); or the Handbook of Therapeutic Antibodies (S. Dubel, Ed.), Wiley, Weinheim, 2007 (see for example pages 252-255).

In a particular aspect, the invention relates to a pharmaceutical composition that comprises a compound according to the invention, and which further comprises at least one pharmaceutically acceptable carrier, diluent or excipient and/or adjuvant, and optionally comprises one or more further pharmaceutically active compound.

Suitable formulations and methods for preparing the same will be clear to the skilled person, and for example include preparations preferable for suitable for parenteral administration (e.g. intravenous, intraperitoneal, subcutaneous, intramuscular, intraluminal, intra-arterial, intrathecal intranasal or intrabronchial administration) but also for topical (e.g. intra-articular, transdermal or intradermal) administration.

Preparations for topical or parenteral administration may for example be sterile solutions, suspensions, dispersions or emulsions that are suitable for infusion or injection. Suitable carriers or diluents for such preparations for example include those mentioned on page 143 of WO 08/020079. Usually, aqueous solutions or suspensions will be preferred.

The invention also provides in a further aspect a kit comprising at least one compound according to the invention. It is contemplated that the kit may be offered in different forms.

The invention will now be further described by means of the following non-limiting preferred aspects, examples and figures.

### 6 EXAMPLES

The following examples illustrate the methods of the disclosure and products of the invention. A series of novel medicated TLR7 agonists were designed, chemically prepared and biologically evaluated as outlined below in more detail. Unexpectedly, compared to prior art compounds such as R484 and some recently reported TLR7/8 agonists (see FIG. 2), several of the novel TLR7 agonists not only gave low nanomolar activities in the cellular TLR7 experiments, but also exhibited a high-selectivity between TLR7 and TLR8. More surprisingly, these novel high-selective TLR7 agonists can initiate a high cytokine and chemokine (IL-6, IL1-b and TNF-α) production and induce strong upregulation of CD40 and CD86 in a cellular monocytes experiment.

### 6.1 Chemical Synthesis of TLR7 Agonists

### Chemical Synthesis of Example 1

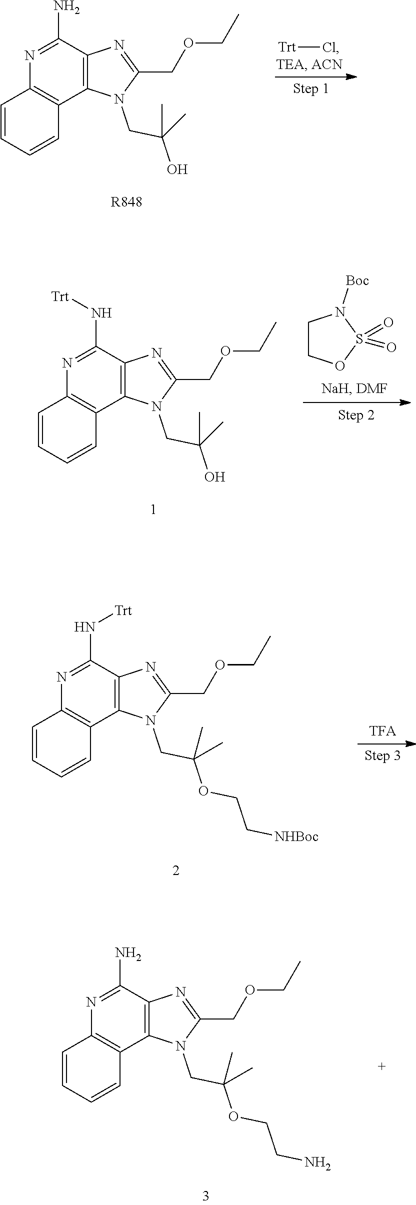

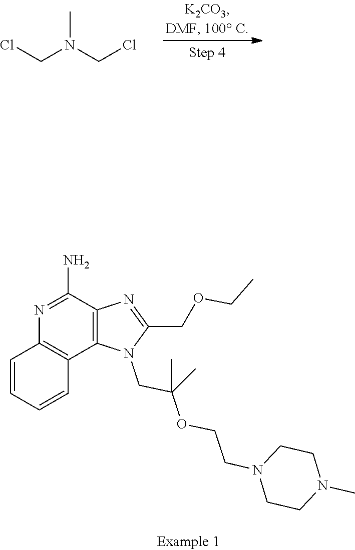

### Step 1

To a stirred solution of R848 (750.00 mg; 2.36 mmol; 1.00 eq.) in acetonitrile (15.00 ml; 20.00 V) was added to triethylamine (0.93 ml; 7.09 mmol; 3.00 eq.) followed by chloro-(diphenyl)methyl]benzene (1.33 g; 4.72 mmol; 2.00 eq.) under nitrogen atmosphere. The resultant suspension was irradiated in a microwave reactor at 100° C. for 1.15 h. The reaction was monitored by TLC and then solvent was removed under vacuum to get a crude. The crude product was purified by flash column chromatography in 100-200 silica gel with Ethyl acetate and Hexane. The required product was eluted in 25-30% of Ethyl acetate and Hexane and eluents were concentrated to afford compound 1 (1.30 g; 2.31 mmol; 97.8%; white solid; purified product).

Yield: 1.30 g (2.31 mmol), % Yield: 97.8.

### Analytical Data:

LCMS: COLUMN: Atlantis dC18 (50×4.6 mm, 5μ), +ve mode; Mobile phase: A: 0.1% HCOOH in H₂O B: ACN; Flow Rate: 1.5 ml/min

RT (min): 2.13; Purity: 98.94%; M+H: 557.10.

### Step 2:

To a stirred solution of sodium hydride (179.63 mg; 4.49 mmol; 2.50 eq.) in DMF (10.00 ml) was added a solution of compound 1 (1.00 g; 1.80 mmol; 1.00 eq.) dissolved in DMF (3 mL). The reaction mixture was stirred at 0° C. under nitrogen atmosphere for 30 min followed by addition of tert-butyl 1,2,3-oxathiazolidine-3-carboxylate 2,2-dioxide (441.12 mg; 1.98 mmol; 1.10 eq.). The reaction mixture was stirred at RT for 12 h and the reaction progress was monitored by LCMS. After completion, the reaction mixture was quenched with cold water and extracted with ethyl acetate (2×100 ml). Combined organic layers were washed with brine and dried over sodium sulphate, filtered and concentrated to get crude. The crude was purified by silica gel column chromatography. Desired product was eluted in 60% ethyl acetate in pet-ether. The combined pure fractions were concentrated under reduced pressure to get compound 2 (550.00 mg; 0.75 mmol; 41.9%; off-white gum; purified product).

Yield: 550.00 mg (0.75 mmol), % Yield: 41.9.

### Analytical Data:

LCMS: COLUMN: Atlantis dC18 (50×4.6 mm, 5μ), +ve mode; Mobile phase: A: 0.1% HCOOH in H₂O B: ACN; Flow Rate: 1.5 ml/min

RT (min): 2.60; Purity: 95.70%; M+H: 700.00.

### Step 3:

To a stirred solution of compound 2 (550.00 mg; 0.75 mmol; 1.00 eq.) in DCM (10.00 ml) was added TFA (0.08 ml; 1.03 mmol; 1.37 eq.) at 0° C. The reaction mixture was stirred at RT for 3 h. The reaction progress was monitored by LCMS. After completion, the reaction mixture was concentrated under reduced pressure. The crude was washed with diethyl ether (2×25 ml), dried under reduced pressure to get compound 3 as trifluoroacetic acid salt (360.00 mg; 0.73 mmol; 97.4%; pale white solid; purified product)

Yield: 360.00 mg (0.73 mmol), % Yield: 97.4.

### Analytical Data:

LCMS: COLUMN: Atlantis dC18 (50×4.6 mm, 5μ), +ve mode; Mobile phase: A: 0.1% HCOOH in H₂O B: ACN; Flow Rate: 1.5 ml/min

RT (min): 0.94; Purity: 95.90%; M+H: 358.00.

### Step 4:

To a stirred solution of compound 3 trifluoroacetic acid salt (200.00 mg; 0.41 mmol; 1.00 eq.) and bis(2-chloroethyl)(methyl)amine hydrochloride (0.16 g; 0.81 mmol; 2.00 eq.) in DMF (10.00 ml) was added potassium carbonate (112.45 mg; 0.81 mmol; 2.00 eq.). The reaction mixture was heated to 100° C. and stirred for 16 h. The reaction progress was monitored by LCMS. After completion, the reaction mixture was partitioned between water and ethyl acetate. The separated organic layer was washed with brine solution, filtered and the filtrate was concentrated under reduced pressure. Crude residue was purified by preparative HPLC using 0.1% NH₄OAc in water and acetonitrile as an eluent required fractions were lyophilized to get Example 1 (4.50 mg; 0.01 mmol; 2.4%; brown gummy solid; purified product).

Yield: 4.50 mg (0.01 mmol), % Yield: 2.4.

### Analytical Data:

LCMS: COLUMN: Atlantis dC18 (50×4.6 mm, 5μ), +ve mode; Mobile phase: A: 0.1% HCOOH in H₂O B: ACN; Flow Rate: 1.5 ml/min

RT (min): 1.76; Purity: 95.81%; M+H: 441.30.

HPLC: Column: XBridge C8, 3.5 μm, 4.6×50 mm; Solvent A: water+0.1% TFA; Solvent B: ACN+0.1% TFA; Flow: 2 ml/min; Gradient: 0 min: 5% B, 8 min: 100% B, 8.1 min: 100% B, 8.5 min: 5% B, 10 min 5% B.

RT (min): 1.72; Purity: 96.98% (max); Purity: 88.09% (220 nm).

1H NMR (400 MHz, DMSO-d6): 9.12 (s, 2H), 8.55 (d, J=8.00 Hz, 1H), 7.80 (d, J=8.00 Hz, 1H), 7.71 (t, J=8.00 Hz, 1H), 7.54 (t, J=7.20 Hz, 1H), 4.95 (s, 4H), 3.53 (m, 4H), 3.36 (m, 4H), 2.73-3.29 (m, 4H), 2.51 (s, 3H), 1.22 (s, 6H), 1.16 (t, J=6.80 Hz, 3H).

### Chemical Synthesis of Example 2

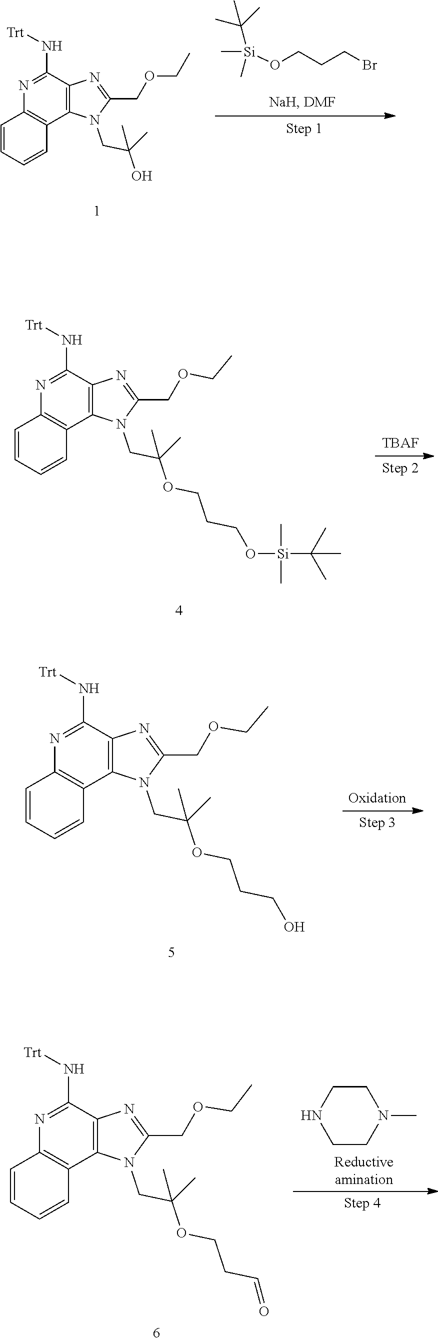

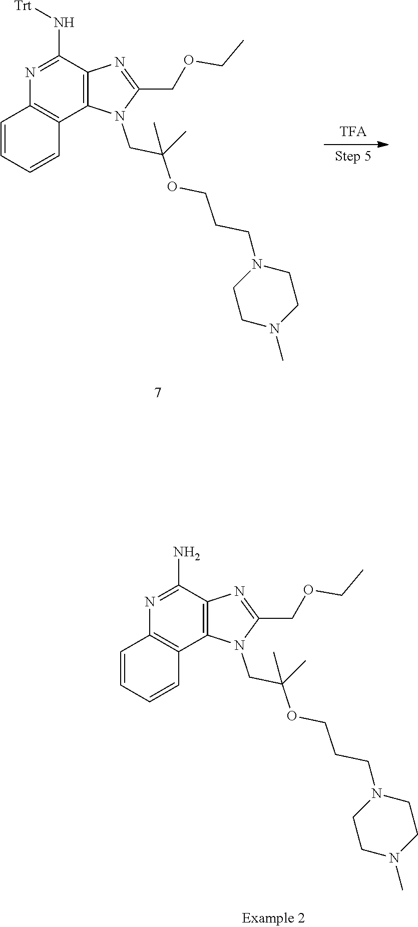

### Step 1:

To a stirred solution of sodium hydride (60%) (158.00 mg; 3.95 mmol; 2.59 eq.) in DMSO (9.00 ml; 10.00 V) at 0° C. under nitrogen atmosphere was added dropwise compound 1 (900.00 mg; 1.53 mmol; 1.00 eq.) in DMSO (5 ml) and reaction mixture was stirred at RT for 30 min. Then (3-bromopropoxy) (tert-butyl)dimethylsilane (0.78 ml; 4.77 mmol; 3.12 eq.) was added at 0° C. and reaction mixture was allow to stir at RT for 3 h under nitrogen atmosphere. The reaction mixture was monitored by LCMS. After completion, The reaction mixture was quenched with ice water at 0° C. and diluted with ethyl acetate. The organic layer was separated, dried over sodium sulphate, filtered and concentrated under vacuum. Crude residue was purified by silica gel chromatography. Required product was eluted with 15% ethyl acetate in hexane to afford compound 4 (215.00 mg; 0.28 mmol; 18.4%; gum; purified product).

Yield: 215.00 mg (0.28 mmol), % Yield: 18.4.

### Analytical Data:

LCMS: COLUMN: Atlantis dC18 (50×4.6 mm, 5μ), +ve mode; Mobile phase: A: 0.1% HCOOH in H₂O B: ACN; Flow Rate: 1.5 ml/min

RT (min): 3.59; Purity: 95.53%; M+H: 729.30.

### Step 2:

To a stirred solution of compound 4 (180.00 mg; 0.24 mmol; 1.00 eq.) in THF (10.00 ml; 55.56 V) was added TBAF (1.0M in THF) (1.00 ml; 1.00 mmol; 4.24 eq.) at 0° C. under nitrogen atmosphere. The reaction mixture was stirred at RT for 3 h and the completion of the reaction was confirmed by TLC. The reaction mixture was quenched with water and extracted with ethyl acetate. The organic layer was collected and concentrated under vacuum to obtain compound 5 (150.00 mg; 0.24 mmol; 100%, gum; purified product)

Yield: 150.00 mg (0.24 mmol), % Yield: 100.

### Analytical Data:

LCMS: COLUMN: Atlantis dC18 (50×4.6 mm, 5μ), +ve mode; Mobile phase: A: 0.1% HCOOH in H₂O B: ACN; Flow Rate: 1.5 ml/min

RT (min): 2.22; Purity: 99.51%; M+H: 615.00.

### Step 3:

To a stirred solution of compound 5 (160.00 mg; 0.26 mmol; 1.00 eq.) in DCM (3.30 ml; 20.63 V) was added 1,1,1-tris(acetyloxy)-1,1-dihydro-1,2-benziodoxol-3-(1H)-one (0.34 g; 0.77 mmol; 3.00 eq.) at 0° C. and the reaction mixture was stirred at RT for 30 min. The completion of the reaction was confirmed by TLC. The reaction mixture was washed with 10% NaHCO₃ solution. Organic layer was collected and concentrated under vacuum to obtained compound 6 (170.00 mg; 0.21 mmol; 81.8%; colourless liquid; crude product)

Yield: 170.00 mg (0.21 mmol), % Yield: 81.8.

### Analytical Data:

LCMS: COLUMN: Atlantis dC18 (50×4.6 mm, 5μ), +ve mode; Mobile phase: A: 0.1% HCOOH in H₂O B: ACN; Flow Rate: 1.5 ml/min

RT (min): 2.36; Purity: 75.96%; M+H: 613.00.

### Step 4:

To a stirred solution of compound 6 (170.00 mg; 0.21 mmol; 1.00 eq.) in 1,2-dichloroethane (10.00 ml; 58.82 V) was added 1-methyl piperazine (0.10 ml; 0.96 mmol; 4.55 eq.) at RT. The reaction mixture was stirred for 10 min at RT and sodium triacetoxyborohydride (300.00 mg; 1.34 mmol; 6.38 eq.) was added to the reaction mixture at 0° C. The reaction mixture was stirred at RT for 30 min. The completion of the reaction was confirmed by TLC. The reaction mixture was quenched with water and extracted with DCM. The organic layer was collected and concentrated under vacuum to obtained compound 7 (140.00 mg; 0.15 mmol; 71.7%; gum; crude product).

Yield: 140.00 mg (0.15 mmol), % Yield: 71.7.

### Analytical Data:

LCMS: COLUMN: Atlantis dC18 (50×4.6 mm, 5μ), +ve mode; Mobile phase: A: 0.1% HCOOH in H₂O B: ACN; Flow Rate: 1.5 ml/min

RT (min): 1.75; Purity: 75.26%; M+H: 697.00.

### Step 5:

To a stirred solution of compound 7 (100.00 mg; 0.11 mmol; 1.00 eq.) in DCM (5.00 ml; 50.00 V) was added TFA (0.90 ml; 11.60 mmol; 9.00 V) at 0° C. The resulting solution was warm to RT and stirred for 3 h. The reaction was monitored by LCMS and then solvent was removed under reduced pressure at below 35° C. to get crude. Crude was purified by preparative HPLC in HCOOH method and the required fractions were lyophilized to afford Example 2 as a formic acid salt (32.91 mg; 0.07 mmol; 60.7%; off-white gum; purified product).

Yield: 32.91 mg (0.07 mmol), % Yield: 60.7.

### Analytical Data:

LCMS: COLUMN: Atlantis dC18 (50×4.6 mm, 5μ), +ve mode; Mobile phase: A: 0.1% HCOOH in H₂O B: ACN; Flow Rate: 1.5 ml/min

RT (min): 1.03; M+H: 455.20.

HPLC: Column: XBridge C8, 3.5 μm, 4.6×50 mm; Solvent A: water+0.1% TFA; Solvent B: ACN+0.1% TFA; Flow: 2 ml/min; Gradient: 0 min: 5% B, 8 min: 100% B, 8.1 min: 100% B, 8.5 min: 5% B, 10 min 5% B.

RT (min): 9.52; Purity: 99.83% (220 nm).

¹HNMR (400 MHz, DMSO-d6): 8.36 (dd, J=8.00, Hz, 1H), 8.15 (s, 1H), 7.62 (dd, J=0.80, 8.20 Hz, 1H), 7.45 (t, J=7.20 Hz, 1H), 7.25 (t, J=6.80 Hz, 1H), 6.91 (s, 2H), 4.72 (s, 4H), 3.54-3.53 (m, 2H), 3.24-3.22 (m, 2H), 2.76-2.75 (m, 2H), 2.52-2.50 (m, 11H), 2.10 (m, 2H), 1.42-1.38 (m, 2H), 1.24-1.12 (m, 9H).

### Chemical Synthesis of Example 3

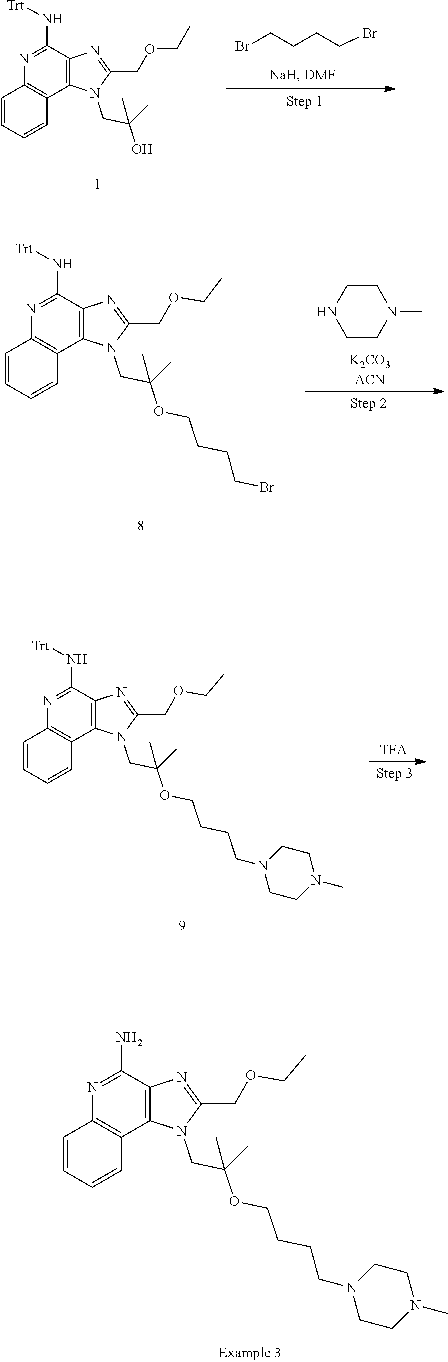

### Step 1:

To a stirred solution of sodium hydride (60%) (0.70 g; 17.50 mmol; 2.41 eq.) in DMSO (40.00 ml; 9.76 V) was added compound 1 (4.10 g; 7.26 mmol; 1.00 eq.) in DMSO (7 ml) at RT under nitrogen atmosphere. The reaction mixture was stirred at RT for 30 min. 1,4-dibromobutane (4.83 ml; 39.46 mmol; 5.43 eq.) was added at RT and reaction mixture was allow to stir at RT for 3 h under nitrogen atmosphere. The reaction progress was monitored by LCMS. The reaction mixture was quenched with ice water at 0° C. The reaction mixture was diluted with ethyl acetate. The organic layer was separated, dried with sodium sulphate, filtered, concentrated under vacuum to get a crude residue. The crude residue was purified by silica gel chromatography. Product was eluted with 15% ethyl acetate in hexane to afford compound 8 (145.00 mg; 0.14 mmol; 2.0%; white gum; purified product) and unreacted starting material was eluted with 45% ethyl acetate in pet-ether to afford compound 1 (3.20 g; 5.12 mmol; 70.4%; white solid; purified product).

Yield: 145 mg (0.14 mmol), % Yield: 2.0.

### Analytical Data:

LCMS: COLUMN: Atlantis dC18 (50×4.6 mm, 5μ), +ve mode; Mobile phase: A: 0.1% HCOOH in H₂O B: ACN; Flow Rate: 1.5 ml/min

RT (min): 2.75; Purity: 68.10%; M+H: 690.90.

### Step 2:

To a stirred solution of compound 8 (470.00 mg; 0.67 mmol; 1.00 eq.) and 1-methyl piperazine (0.08 ml; 0.74 mmol; 1.10 eq.) in acetonitrile (14.10 ml; 30.00 V) was added potassium carbonate (189.73 mg; 1.35 mmol; 2.00 eq.) at RT. The suspension was heated to 50° C. for 16 h and the reaction was monitored by TLC. After completion, solvent was removed to get a residue. The residue was dissolved in water and extracted with DCM (8 mL). The organic layer was washed with water. The organic layer was dried over sodium sulphate, filtered and concentrated to get a crude material. This material was purified by silica gel column chromatography. The desired product was eluted in 7% MeOH in DCM to get compound 9 (400.00 mg; 0.55 mmol; 82.1%; off-white powder; purified product).

Yield: 400 mg (0.55 mmol), % Yield: 82.1.

### Analytical Data:

LCMS: Column: Atlantis dC18 (50×4.6 mm) 3.5 μm; Solvent A; 0.1% HCOOH in H₂O:ACN (95:5) Solvent B; ACN; Flow Rate: 1.5 ml/min.

RT (min): 1.79; Purity: 98.20%.

### Step 3:

To a stirred solution of compound 9 (95.00 mg; 0.11 mmol; 1.00 eq.) in DCM (4.75 ml; 50.00 V) was added TFA (0.36 ml; 4.58 mmol; 40.00 eq.) at 0° C. The resulting solution was warm to RT and stirred for 3 h. The reaction was monitored by LCMS. After completion, solvent was removed under reduced pressure at below 35° C. to get crude. The crude was purified by preparative HPLC in HCOOH method and the required fractions were lyophilized to afford Example 3 as a formic acid salt (44.00 mg; 0.08 mmol; 74.1%; off-white gum; purified product).

Yield: 44.00 mg (0.08 mmol), % Yield: 74.1.

### Analytical Data:

LCMS: Column: Atlantis dC18 (50×4.6 mm) 3.5 μm; Solvent A; 0.1% HCOOH in H₂O:ACN (95:5) Solvent B; ACN; Flow Rate: 1.5 ml/min.

RT (min): 0.99; Purity: 99.05%; M+H: 469.10.

HPLC: Column: Phenomenex Gemini C18 (150*4.6) mm, 3.0 μm; Solvent A: water+10 mM Ammonium acetate: Solvent B: Acetonitrile; Flow Rate: 1.0 ml/min.

RT (min): 7.96; Purity: 99.22% (max); Purity: 97.91% (220 nm).

¹HNMR (400 MHz, DMSO-d6): 8.33 (d, J=8.40 Hz, 1H), 7.59 (d, J=8.40 Hz, 1H), 7.42 (t, J=Hz, 1H), 7.22 (t, J=Hz, 1H), 6.67 (s, 2H), 4.73 (s, 2H), 3.49-3.54 (m, 6H), 3.20 (t, J=9.20 Hz, 4H), 2.078-2.335 (m, 11H), 1.13-1.26 (m, 14H), (s, H), (s, H), (s, H), (s, H).

### Chemical Synthesis of Example 4

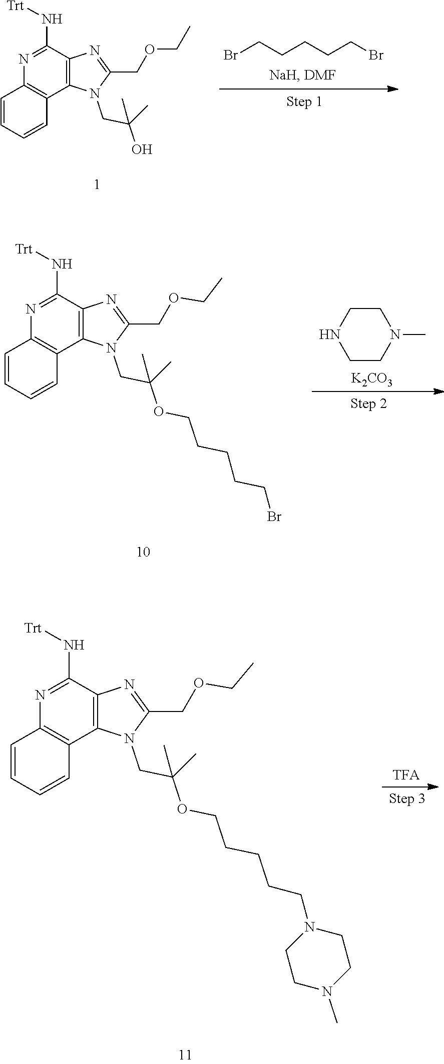

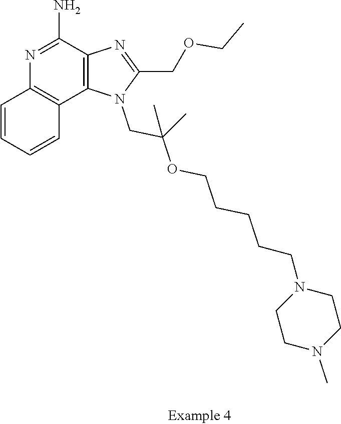 [SMILES: CCOCc1nc2c(N)nc3ccccc3c2n1CC(C)(C)OCCCCCN1CCN(C)CC1]

### Step 1:

To a stirred solution of sodium hydride (104.78 mg; 2.62 mmol; 3.00 eq.) in DMSO (10.00 ml; 20.00 V)) was added a solution of compound 1 (500.00 mg; 0.87 mmol; 1.00 eq.) in DMSO (5 ml) at 0° C. under nitrogen atmosphere. The reaction mixture was stirred for 30 min. Then 1,5-dibromopentane (614.64 mg; 2.62 mmol; 3.00 eq.) was added and resulting solution was stirred at RT for 16 h. The reaction was monitored by LCMS. After completion, reaction mixture was quenched with cold water and extracted with ethyl acetate (2×100 ml). The combined organic layers were washed with brine and dried over sodium sulphate, filtered and concentrated to get crude. The crude was purified by silica gel column chromatography. Desired product was eluted in 16% ethyl acetate in pet-ether. Combined pure fractions were concentrated under reduced pressure to get compound 10 (150.00 mg; 0.21 mmol; 23.7%; off-white gum; purified product).

Yield: 150.00 mg (0.21 mmol), % Yield: 23.7.

### Analytical Data:

LCMS: COLUMN: Atlantis dC18 (50×4.6 mm, 5μ), +ve mode; Mobile phase: A: 0.1% HCOOH in H₂O B: ACN; Flow Rate: 1.5 ml/min

RT (min): 3.32; Purity: 97.45%; M+H: 705.30.

### Step 2:

To a stirred solution of compound 10 (150.00 mg; 0.21 mmol; 1.00 eq.) in Acetonitrile (3.00 ml; 20.00 V) were added potassium carbonate (87.63 mg; 0.62 mmol; 3.00 eq.) and 1-methyl piperazine (0.03 ml; 0.31 mmol; 1.50 eq.) at RT. The suspension was heated to 50° C. and stirred for 16 h. The reaction was monitored by LCMS. After completion, reaction mixture was concentrated under reduced pressure to get a crude residue. The residue was dissolved in DCM (100 ml) and washed with brine (2×50 ml). The organic layer was concentrated under reduced pressure to get crude. The crude was purified by silica gel column chromatography. Desired product was eluted in 7% MeOH in DCM. Combined pure fractions were concentrated under reduced pressure to get compound 11 (70.00 mg; 0.09 mmol; 41.1%; off-white gum; purified product)

Yield: 70.00 mg (0.09 mmol), % Yield: 41.1.

### Analytical Data:

LCMS: COLUMN: Atlantis dC18 (50×4.6 mm, 5μ), +ve mode; Mobile phase: A: 0.1% HCOOH in H₂O B: ACN; Flow Rate: 1.5 ml/min

RT (min): 1.72; Purity: 88.13%; M+H: 725.20.

### Step 3:

To a stirred solution of compound 11 (70.00 mg; 0.08 mmol; 1.00 eq.) in DCM (3.50 ml; 50.00 V) was added TFA (0.26 ml; 3.32 mmol; 40.00 eq.) at 0° C. The resulting solution was stirred for 3 h. The reaction was monitored by LCMS. After completion, solvent was removed under reduced pressure at below 35° C. to get a crude material. This material was purified by preparative HPLC in HCOOH method. The required fractions were lyophilized to afford Example 4 as a formic acid salt (32.00 mg; 0.06 mmol; 72.6%; pale yellow gum; purified product).

Yield: 32.00 mg (0.06 mmol), % Yield: 72.6.

### Analytical Data:

LCMS: Column: Atlantis dC18 (50×4.6 mm) 3.5 μm; Solvent A; 0.1% HCOOH in H₂O:ACN (95:5) Solvent B; ACN; Flow Rate: 1.5 ml/min.

RT (min): 1.09; Purity: 99.24%; M+H: 483.10.

HPLC: Column: Phenomenex Gemini C18 (150*4.6) mm, 3.0 μm; Solvent A: 0.1% TFA in H₂O:ACN (95:5), Solvent B: ACN Flow Rate: 1.0 ml/min.

RT (min): 8.82; Purity: 99.42% (max); Purity: 98.26% (220 nm).

1HNMR (400 MHz, DMSO-d6): δ 8.57 (d, J=7.60 Hz, 1H), 8.37 (s, 1H), 7.71-7.79 (m, 1H), 7.67-7.71 (m, 1H), 7.52-7.56 (m, 1H), 4.90 (s, 4H), 3.64 (s, 4H), 3.28-3.33 (m, 8H), 2.86 (s, 3H), 2.43-2.47 (m, 2H), 1.21-1.32 (m, 12H), 0.96 (d, J=4.40 Hz, 2H).

### Chemical Synthesis of Example 5

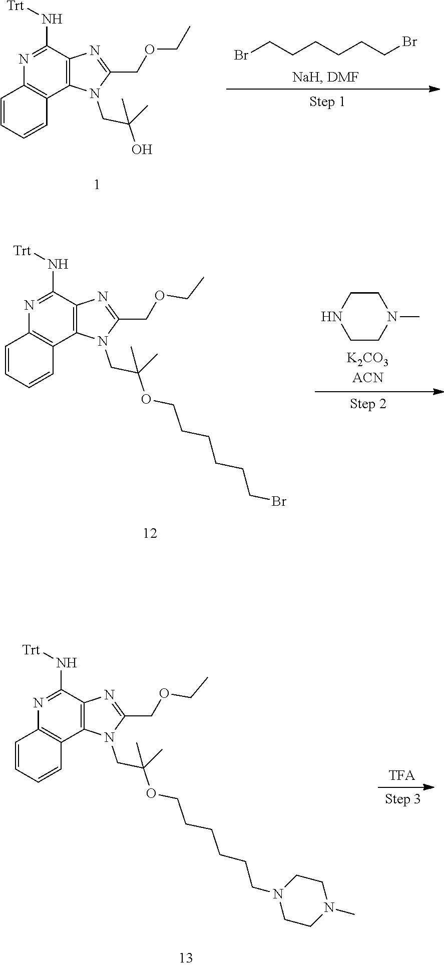

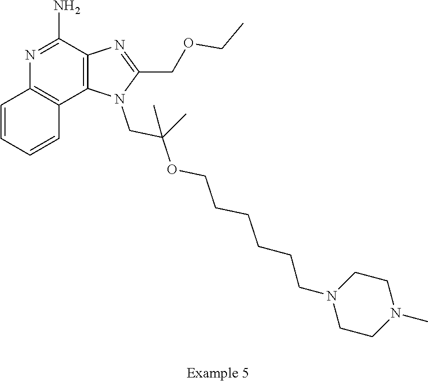 [SMILES: CCOCc1nc2c(N)nc3ccccc3c2n1CC(C)(C)OCCCCCCN1CCN(C)CC1]

### Step 1:

To a stirred solution of sodium hydride (60%) (122.25 mg; 3.06 mmol; 2.50 eq.) in DMSO (7.00 ml; 10.00 V) at 0° C. was added dropwise compound 1 (700.00 mg; 1.22 mmol; 1.00 eq.) in DMSO (5 ml) and the reaction mixture was stirred at RT for 30 min. Then 1,6-dibromohexane (0.58 ml; 3.67 mmol; 3.00 eq.) was added to the reaction mixture at 0° C. and the reaction mixture was allow to stir at RT for 16 h under nitrogen atmosphere. The reaction progress was monitored by LCMS. The reaction mixture was quenched with ice water at 0° C. The reaction mixture was diluted with ethyl acetate and organic layer was separated, dried over sodium sulphate, filtered, concentrated under vacuum. The crude residue was purified by silica gel chromatography. The required product was eluted with 15% ethyl acetate in hexane to afford compound 12 (200.00 mg; 0.21 mmol; 17.1%; off-white oil; purified product).

Yield: 200.00 mg (0.21 mmol), % Yield: 17.1.

### Analytical Data:

LCMS: COLUMN: Atlantis dC18 (50×4.6 mm, 5μ), +ve mode; Mobile phase: A: 0.1% HCOOH in H₂O B: ACN; Flow Rate: 1.5 ml/min

RT (min): 3.26; Purity: 75.40%; M+H: 719.00.

### Step 2:

To a stirred solution of compound 12 (200.00 mg; 0.21 mmol; 1.00 eq.) and 1-methyl piperazine (0.05 ml; 0.52 mmol; 2.50 eq.) in Acetonitrile (2.00 ml; 10.00 V) was added Potassium carbonate (88.64 mg; 0.63 mmol; 3.00 eq.) at RT. The suspension was heated to 50° C. and stirred for 16 h. The reaction was monitored by LCMS. After completion, solvent was removed to get residue. The residue was dissolved in water and extracted with DCM (20 ml). The organic layer was washed with water. The organic layer was dried over sodium sulphate, filtered and concentrated to get crude residue. The residue was purified by silica gel chromatography. Required product was eluted with 8% MeOH in DCM to obtained compound 13 (110.00 mg; 0.14 mmol; 67.6%; white solid; purified product).

Yield: 110.00 mg (0.14 mmol), % Yield: 67.6.

### Analytical Data:

LCMS: COLUMN: Atlantis dC18 (50×4.6 mm, 5μ), +ve mode; Mobile phase: A: 0.1% HCOOH in H₂O B: ACN; Flow Rate: 1.5 ml/min

RT (min): 1.79; Purity: 95.17%; M+H: 739.00.

### Step 3:

To a stirred solution of compound 13 (110.00 mg; 0.14 mmol; 1.00 eq.) in DCM (5.50 ml; 50.00 V) was added TFA (0.44 ml; 5.67 mmol; 40.00 eq.) at 0° C. The resulting solution was stirred for 3 h. The reaction progress was monitored by LCMS. After completion, solvent was removed under reduced pressure at below 35° C. to get crude. The crude was purified by Preparative HPLC in HCOOH method. The required fractions were lyophilized to afford Example 5 as a formic acid salt (60.00 mg; 0.11 mmol; 77.1%; white solid; purified product).

Yield: 60.00 mg (0.11 mmol), % Yield: 77.1.

### Analytical Data:

LCMS: Column: Atlantis dC18 (50×4.6 mm) 3.5 μm; Solvent A; 0.1% HCOOH in H₂O: ACN (95:5) Solvent B; ACN; Flow Rate: 1.5 ml/min.

RT (min): 1.17; Purity: 98.84%; M+H: 497.10.

HPLC: Column: XBridge C8, 3.5 μm, 4.6×50 mm; Solvent A: water+0.1% TFA; Solvent B: ACN+0.1% TFA; Flow: 2 ml/min; Gradient: 0 min: 5% B, 8 min: 100% B, 8.1 min: 100% B, 8.5 min: 5% B, 10 min 5% B.

RT (min): 5.39; Purity: 99.49% (max); Purity: 99.54% (220 nm).

¹HNMR (400 MHz, MeOD): δ 8.60 (d, J=8.40 Hz, 1H), 7.80-7.71 (m, 2H), 7.61-7.57 (m, 1H), 4.87-4.84 (m, 6H), 3.67-3.62 (m, 2H), 3.34-3.15 (m, 10H), 2.84-2.79 (m, 5H), 1.53-1.49 (m, 2H), 1.31-1.22 (m, 11H), 1.16-1.09 (m, 4H).

### Chemical Synthesis of Example 6

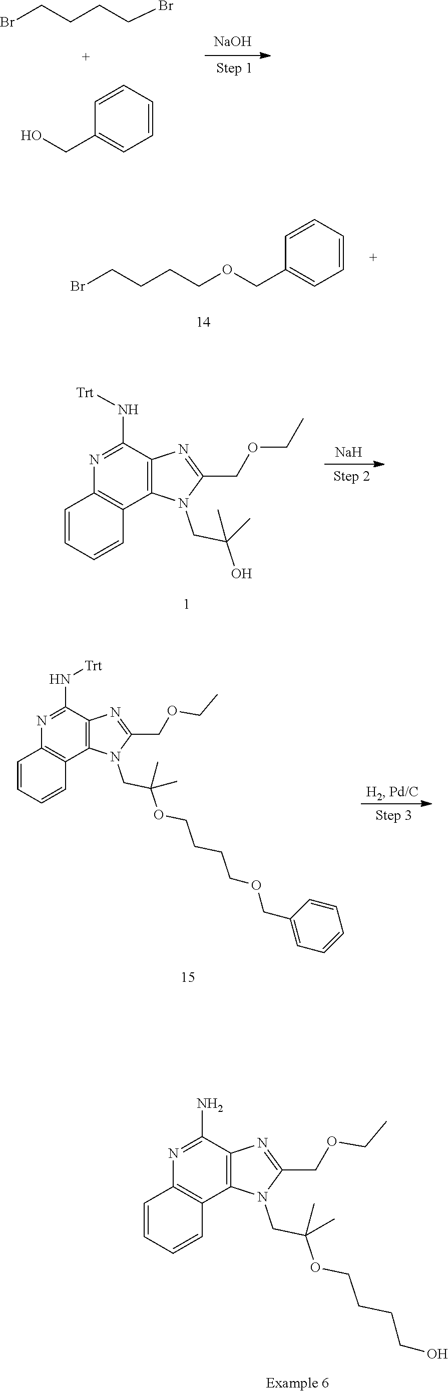

### Step 1:

To a stirred solution of benzyl alcohol (1.00 g; 9.20 mmol; 1.00 eq.) in sodium hydroxide 10% aqueous solution (0.92 g; 23.00 mmol; 2.50 eq.) dissolved in water (20.00 ml; 20.00 V) were added tetra butyl-ammonium; hydrogen sulfate (64.42 mg; 0.18 mmol; 0.02 eq.) followed by 1,4-dibromobutane (3.99 g; 18.4 mmol; 2 eq.) at 0° C. The resulting mixture was stirred at 70° C. for 4 h and the reaction was monitored by TLC. After completion, the reaction mixture was diluted with water and extracted with ethyl acetate. The combined organic layers were washed with brine and dried over sodium sulphate, filtered and concentrated to get a crude material. This material was purified by flash column chromatography in 100-200 silica gel with ethyl acetate and hexane. The required product was eluted in 1-5% of ethyl acetate and hexane and eluents were concentrated to afford compound 14 (1.00 g; 4.05 mmol; 44.0%; colorless oil; purified product)

Yield: 1 g (4.05 mmol), % Yield: 44.

### Analytical Data:

¹HNMR (400 MHz, DMSO-d6): δ 7.40-7.29 (m, 5H), 4.53 (s, 2H), 3.53 (t, J=−12.40 Hz, 2H), 3.46 (t, J=6.80 Hz, 2H), 2.04-1.79 (m, 4H).

### Step 2:

To a stirred suspension of sodium hydride (60%) (179.63 mg; 4.49 mmol; 2.50 eq.) in DMSO (20.00 ml; 20.00 V) at 0° C. under nitrogen atmosphere. was added dropwise compound 1 in DMSO (5 mL) and the reaction mixture was stirred at RT for 30 min. Then [(4-bromobutoxy)methyl]benzene (882.33 mg; 3.59 mmol; 2.00 eq.) was added at 0° C. and reaction mixture was stirred at RT for 16 h under nitrogen atmosphere. The reaction mixture was monitored by TLC. After completion, the reaction mixture was quenched with ice water and extracted with ethyl acetate. The combined organic layers were washed with brine and dried over sodium sulphate, filtered and concentrated to get a crude material. This material was purified by flash column chromatography in 100-200 silica gel with ethyl acetate and hexane. The required product was eluted in 10-15% of ethyl acetate and hexane and eluents were concentrated to afford compound 15 (330.00 mg; 0.45 mmol; 24.8%; off-white gummy; purified product)

Yield: 330 mg (0.45 mmol), % Yield: 24.8.

### Analytical Data:

LCMS: COLUMN: XBridge C8 (50×4.6 mm) 3.5 μm; Solvent A: 0.1% TFA in H₂O:ACN (95:5), Solvent B: ACN; Flow Rate: 1.5 ml/min.

RT (min): 3.35; Purity: 97.21%; M+H: 719.3.

### Step 3:

To a stirred solution of compound 15 (100.00 mg; 0.14 mmol; 1.00 eq.) in ethanol (5.00 ml) and AcOEt (5.00 ml) was added palladium on carbon (100.00 mg; 0.09 mmol; 0.70 eq.) at RT. The resulting reaction mixture was stirred at RT under 5 kg hydrogen pressure in autoclave. After completion, the reaction mixture was filtered through celite. The filtrate was concentrated under reduced pressure to get a crude material. This material was purified by preparative HPLC using 0.1% HCOOH in water and ACN as eluents. The combined pure fractions were lyophilized to get Example 6 as a formic acid salt (20.00 mg; 0.05 mmol; 37.9%; off white solid; purified product).

Yield: 20 mg (0.05 mmol), % Yield: 37.9.

### Analytical Data:

LCMS: Column: Atlantis dC18 (50×4.6 mm) 3.5 μm; Solvent A: 0.1% HCOOH in H₂O:ACN (95:5) Solvent B; ACN; Flow Rate: 1.5 ml/min.

RT (min): 1.46; Purity: 99.69%; M+H: 387.1

HPLC: Column: Atlantis dC18 (50×4.6 mm) 3.5 μm; Solvent A: 0.1% HCOOH in H₂O:ACN (95:5) Solvent B; ACN

RT (min): 7.01; Purity (max): 99.86%; Purity (220 nm): 99.32%.

¹HNMR (400 MHz, DMSO-d6): δ 0.00 (s, 1H), 7.58-7.60 (m, 1H), 7.39-7.43 (m, 1H), 7.20-7.24 (m, 1H), 4.77 (s, 5H), 3.52 (d, J=7.20 Hz, 2H), 3.35 (t, J=6.00 Hz, 2H), 3.21 (s, 1H), 1.32 (q, J=2.00 Hz, 4H), 1.26 (q, J=10.80 Hz, 4H), 1.14 (q, J=7.20 Hz, 4H).

### Chemical Synthesis of Example 7

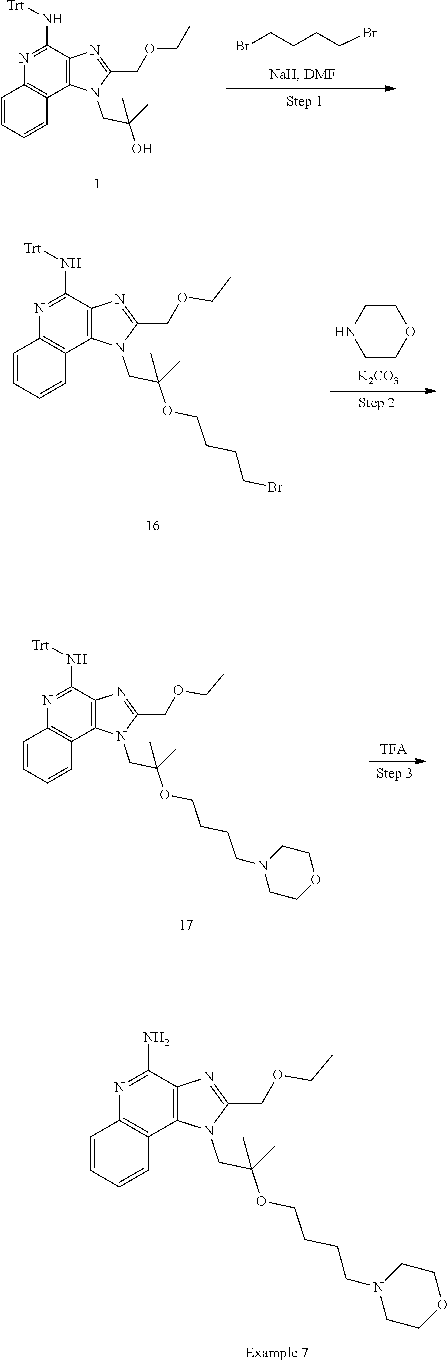

### Step 1:

To a suspension of sodium hydride (60%) (0.21 g; 5.31 mmol; 2.30 eq.) in DMSO (13.00 ml; 10.00 V), a solution of compound 1 (1.30 g; 2.31 mmol; 1.00 eq.) in DMSO (13.00 ml; 10.00 V) was added dropwise at 0° C. under nitrogen atmosphere. It was then stirred at rt for 30 min. 1,4-dibromobutane (0.76 g; 3.46 mmol; 1.50 eq.) was added at 0° C. and the reaction mixture was stirred at rt for additional 1 h. After completion, the reaction mixture was quenched with ice water and diluted with ethyl acetate and the organic layer was separated, dried over sodium sulphate and the solvent was evaporated under reduced pressure. The crude residue was purified by flash chromatography (15% ethyl acetate in hexane) to afford compound 16 (120.00 mg; 0.17 mmol; 7.3%; white solid; purified product).

Yield: 120.00 mg (0.17 mmol), % Yield: 7.3.

### Analytical Data:

LCMS-COLUMN: XBridge C8, 3.5 μm, 4.6×50 mm; Mobile phase: A: 0.1% TFA in H₂O:ACN (95:5); Mobile phase B: 0.1% TFA in ACN; Flow Rate: 1.5 ml/min.

RT (min): 3.28; Purity (max): 97.18%; M+H: 691.20

### Step 2:

Potassium carbonate (35.67 mg; 0.25 mmol; 3.00 eq.) was added to a solution of compound 16 (3, 60.00 mg; 0.08 mmol; 1.00 eq.) and morpholine (22.26 mg; 0.25 mmol; 3.00 eq.) in acetonitrile (1.80 ml; 30.00 V) at rt. Then the suspension was heated to 70° C. and stirred for 20 h and the progress of the reaction was monitored by TLC. After completion, the solvent was evaporated and the residue was dissolved in water. It was then extracted with dichloromethane. The organic layer was washed with water and dried over sodium sulphate and then the solvent was evaporated under reduced pressure. The crude was purified by flash chromatography (60% EtOAc in pet ether) to afford compound 17 (60.00 mg; 0.08 mmol; 97.8%; gum; purified product).

Yield: 60.00 mg (0.08 mmol), % Yield: 97.8.

### Analytical Data:

LCMS-COLUMN: ATLANTIS dC18 (50×4.6 mm) 5 μm; Mobile Phase A: 0.1% HCOOH in Water; B: 0.1% HCOOH in ACN; Flow Rate: 0.8 ml/min.

RT (min): 1.88; Purity (max): 95.95%; M+H: 698.00

### Step 3:

To a stirred solution of compound 17 (5, 60.00 mg; 0.08 mmol; 1.00 eq.) in dichloromethane (3.00 ml; 50.00 V) was added TFA (0.20 ml; 2.58 mmol; 3.33 V) at 0° C. The resulting solution was warmed to rt and stirred for another 3 h. The progress of the reaction was monitored by TLC. After completion, the reaction mixture was concentrated under reduced pressure and the crude product was purified by flash column chromatography (10% methanol in dichloromethane) to afford Example 7 (34.00 mg; 0.07 mmol; 89.5%; off-white gum; Purified Product).

Yield: 34.00 mg (0.07 mmol), % Yield: 89.5.

### Analytical Data:

LCMS-COLUMN: ATLANTIS dC18 (50×4.6 mm) 5 μm; Mobile Phase A: 0.1% HCOOH in Water; B: 0.1% HCOOH in ACN; Flow Rate: 0.8 ml/min.

RT (min): 1.15; Purity (max): 98.94%; M+H: 456.10.

HPLC-Column: ATLANTIS dC18 (50×4.6 mm) 5 mm; Mobile Phase A: 0.1% TFA in MilliQ water; Mobile Phase B: Acetonitrile; Flow Rate: 1.0 ml/min.

RT (min): 9.25; Purity (max): 95.25%; M+H: 456.10; Purity (220 nm): 96.41%.

### Chemical Synthesis of Example 8

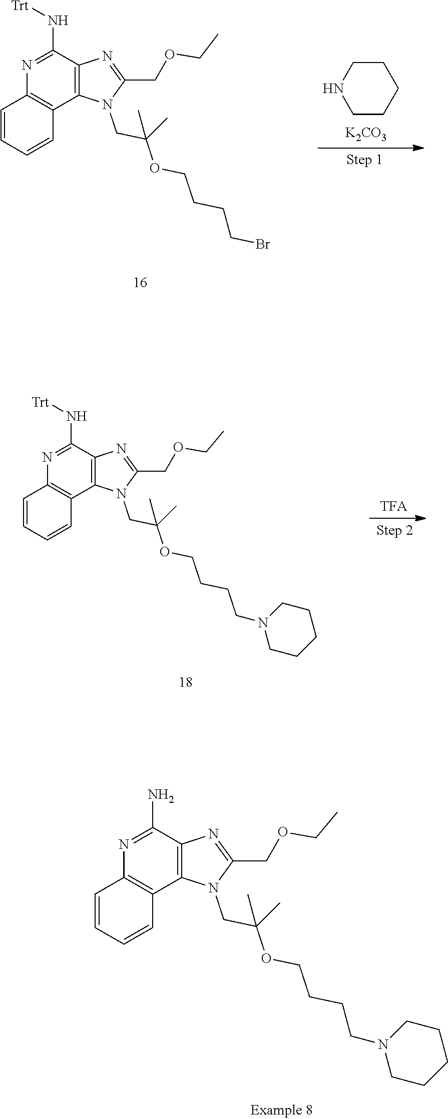

### Step 1:

To a stirred solution of compound 16 (60.00 mg; 0.08 mmol; 1.00 eq.) and piperidine (18.54 mg; 0.21 mmol; 2.50 eq.) in acetonitrile (1.80 ml; 30.00 V) was added potassium carbonate (30.09 mg; 0.21 mmol; 2.50 eq.) at RT. The suspension was heated to 70° C. with stirring for 20 h. The progress of the reaction was monitored by TLC. After completion of the reaction, the solvent was evaporated and the residue was subjected to flash chromatography (5% MeOH in dichloromethane) to afford compound 18 (40.00 mg; 0.06 mmol, 66.9%; gum, purified product).

Yield: 40.00 mg (0.06 mmol), % Yield: 66.9.

### Analytical Data:

LCMS-COLUMN: ATLANTIS dC18 (50×4.6 mm) 5 μm; Mobile Phase: A 0.1% HCOOH in H2O:ACN (95:5), B: ACN Flow Rate: 1.5 ml/min.

RT (min): 2.01; Purity (max): 99.36%; M+H: 696.1

### Step 2:

To a stirred solution of compound 18 (40.00 mg; 0.06 mmol; 1.00 eq.) in dichloromethane (2.00 ml; 50.00 V) was added TFA (0.15 ml; 1.93 mmol; 3.75 V) at 0° C. The reaction mixture was warmed to RT and stirred for another 3 h. The progress of the reaction was monitored by TLC. After completion of the reaction it was concentrated under reduced pressure and the crude was purified by flash column chromatography (15% methanol in dichloromethane) to afford Example 8 (16.00 mg; 0.03 mmol; 61.0%; light brown gum; purified product).

Yield: 16.00 mg (0.03 mmol), % Yield: 61.0.

### Analytical Data:

LCMS-COLUMN: ATLANTIS dC18 (50×4.6 mm) 5 μm; Mobile Phase: A 0.1% HCOOH in H₂O:ACN (95:5), B: ACN Flow Rate: 1.5 ml/min.

RT (min): 1.24; Purity (max): 98.78%; M+H: 454.10.

### Chemical Synthesis of Example 9

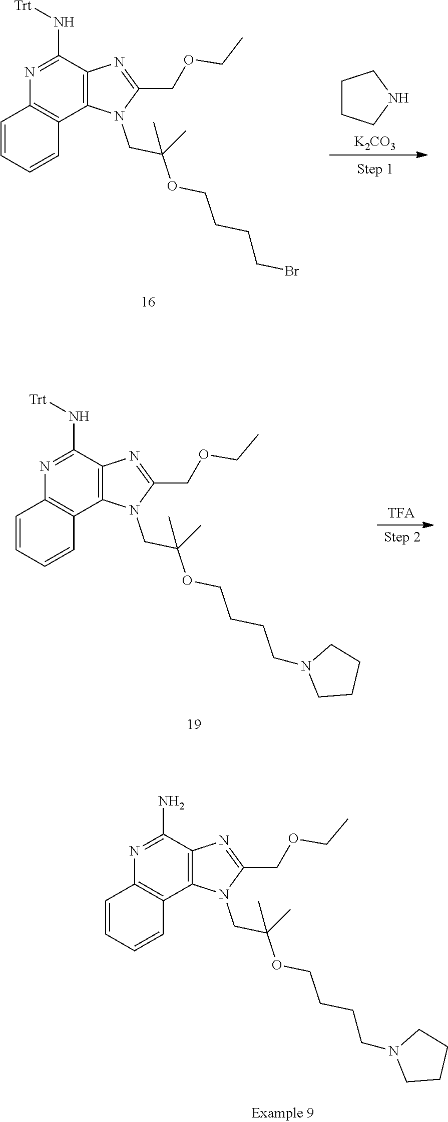

### Step 1:

To a stirred solution of compound 16 (60.00 mg; 0.09 mmol; 1.00 eq.) and pyrrolidine (19.17 mg; 0.26 mmol; 3.00 eq.) in acetonitrile (1.20 ml; 20.00 V) was added potassium carbonate (35.39 mg; 0.26 mmol; 3.00 eq.) at RT. The suspension was heated to 70° C. and stirred for 20 h and the reaction was monitored by TLC. After completion of the reaction, the solvent was removed and the residue was dissolved in water and then extracted with DCM. The organic layer was washed with water and dried over sodium sulphate. The solvent was then evaporated under reduced pressure and the crude product was purified by flash chromatography (60% EtOAc in pet ether) to afford compound 19 (60.00 mg; 0.09 mmol; 99.8%; gum; purified product).

Yield: 60.00 mg (0.09 mmol), % Yield: 99.8.

### Analytical Data:

LCMS-COLUMN: Column: ATLANTIS dC18 (50×4.6 mm) 5 mm; Mobile Phase: A 0.1% HCOOH in H2O:ACN (95:5), B: CAN; Flow Rate: 1.5 ml/min.

RT (min): 1.99; Purity (max): 96.78%; M+H: 682.00.

### Step 2:

To a stirred solution of compound 19 (60.00 mg; 0.08 mmol; 1.00 eq.) in dichloromethane (3.00 ml; 50.00 V) was added TFA (0.15 ml; 1.93 mmol; 2.50 V) at 0° C. The resulting solution was warmed to RT and stirred for 3 h. The reaction was monitored by TLC. After completion, the reaction mixture was concentrated under reduced pressure and the crude product was purified by column chromatography (10% methanol in dichloromethane) to afford Example 9 (20.00 mg; 0.05 mmol; 54.3%; off-white gum; purified product).

Yield: 20.00 mg (0.05 mmol), % Yield: 54.3.

### Analytical Data:

LCMS-COLUMN: ATLANTIS dC18 (50×4.6 mm) 5 mm; Mobile Phase: A 0.1% HCOOH in H₂O:ACN (95:5), B: ACN Flow Rate: 1.5 ml/min.

RT (min): 1.20; Purity (max): 99.56%; M+H: 440.10.

HPLC-Column: ATLANTIS dC18 (50×4.6 mm) 5 mm; Mobile Phase A: 0.1% TFA in MilliQ water; Mobile Phase B: Acetonitrile; Flow Rate: 1.0 ml/min.

RT (min): 5.76; Purity (max): 99.55%; M+H: 456.10; Purity (220 nm): 99.42%.

### Chemical Synthesis of Example 10

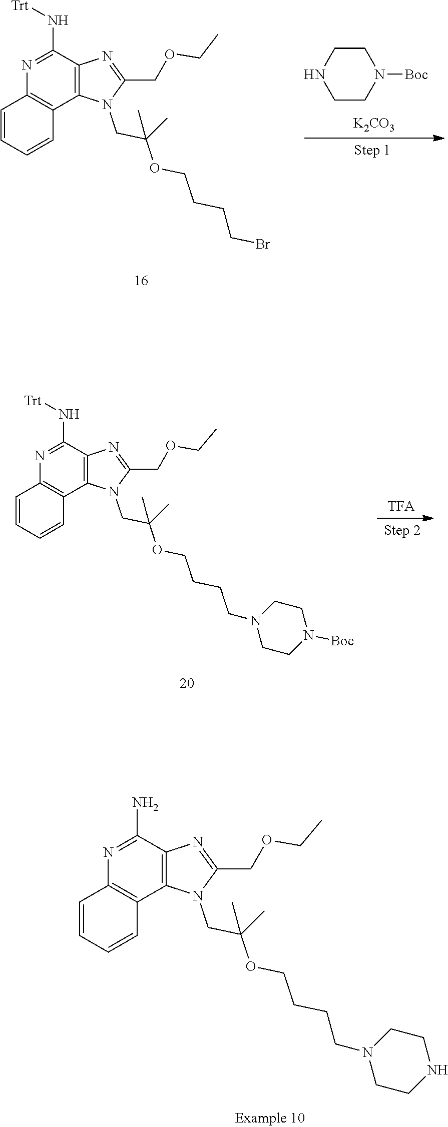

### Step 1:

To a stirred solution of compound 16 (50.0 mg; 0.07 mmol; 1.00 eq.) and piperazine-1-carboxylic acid tert-butyl ester (34.0 mg; 0.18 mmol; 2.50 eq.) in acetonitrile (1.50 ml; 30.00 V) was added potassium carbonate (25.23 mg; 0.18 mmol; 2.50 eq.) at RT. The suspension was heated to 70° C. and stirred for 20 h. The progress of the reaction was monitored by TLC. After completion of the reaction, the solvent was removed and the residue was re-dissolved in water and then extracted with dichloromethane. The organic layer was washed with water and then it was dried over anhydrous sodium sulphate. The solvent was concentrated under reduced pressure and the crude product was purified by flash column chromatography (60% EtOAc in pet ether) to afford compound 20 (45 mg; 0.06 mmol; 78.5%; gum, purified product).

Yield: 45.00 mg (0.06 mmol), % Yield: 78.5.

### Analytical Data:

LCMS-Column: Atlantis dC18 (50×4.6 mm, 5 μm), +ve mode; 0.1% HCOOH in H₂O, B: ACN; Flow Rate: 1.5 ml/min.

RT (min): 2.07; Purity (max): 99.44%; M+H: 797.00

### Step 2:

TFA (0.15 ml; 1.93 mmol; 3.33 V) was added to a stirred solution of compound 20 (3, 45.00 mg; 0.06 mmol; 1.00 eq.) in dichloromethane (2.25 ml; 50.00 V) at 0° C. The resulting solution was warmed to RT and stirred for 3 h. The reaction was monitored by LCMS. After completion, the solvent was removed under reduced pressure at below 35° C. and the crude product was purified by preparative HPLC. The product fractions were lyophilized to afford Example 10 as a formic acid salt (25.00 mg; 0.05 mmol; 88.1%; off-white gum; purified product).

Yield: 25.00 mg (0.05 mmol), % Yield: 88.1.

### Analytical Data:

LCMS-Column: Atlantis C18 (50×4.6 mm, 5 μm); Solvent A: water+0.1% TFA; Solvent B: ACN+0.1% TFA; Flow: 2 ml/min.

RT (min): 9.46; Purity (max): 98.13%; M+H: 455.30.

HPLC-Column: Atlantis dC18 (50×4.6 mm, 5 μm), +ve mode; Solvent A: water+0.1% TFA; Solvent B: ACN+0.1% TFA; Flow: 2 ml/min; Gradient: 0 min: 5% B, 8 min: 100% B, 8.1 min: 100% B, 8.5 min: 5% B, 10 min 5% B., Flow Rate: 0.8 mL/min.

RT (min): 8.47; Purity (max): 99.05%; Purity (220 nm): 98.46%.

¹H NMR (400 MHz, MeOD): □□8.56 (d, J=8.40 Hz, 1H), 8.44 (s, 1H), 7.78 (d, J=0.52 Hz, 1H), 7.69-7.65 (m, 1H), 7.53-7.49 (m, 1H), 4.91 (m, 4H), 3.67-3.61 (m, 2H), 3.18-3.15 (m, 4H), 2.50 (m, 4H), 2.15-2.06 (m, 2H), 1.32 (m, 6H), 1.28-1.25 (m, 5H), 1.14-1.11 (m, 2H).

### Chemical Synthesis of Example 11

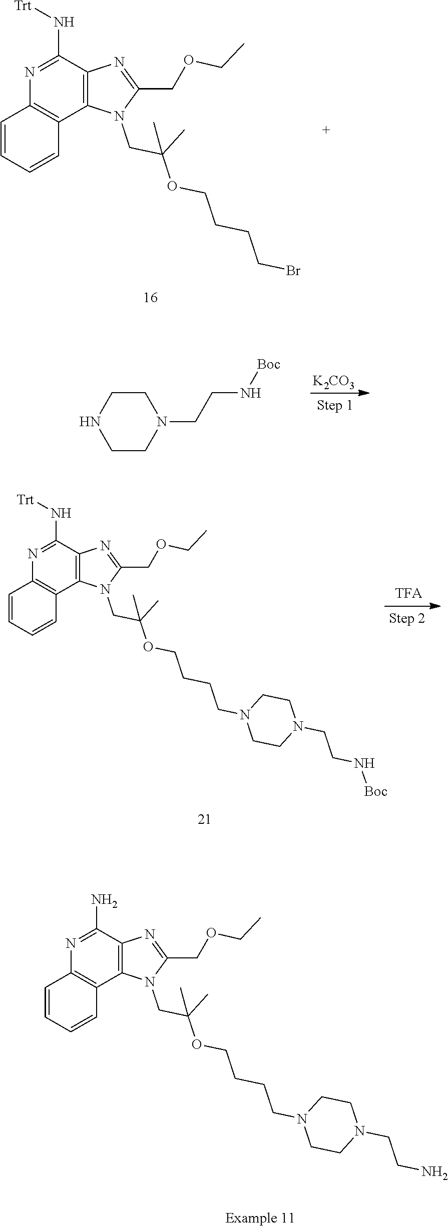

### Step 1:

To a stirred solution of compound 16 (145.00 mg; 0.14 mmol; 1.00 eq.) and (2-piperazin-1-yl-ethyl)-carbamic acid tert-butyl ester (136.40 mg; 0.57 mmol; 4.00 eq.) in acetonitrile (4.35 ml; 30.00 V) was added potassium carbonate (60.40 mg; 0.43 mmol; 3.00 eq.) at RT. The suspension was heated to 80° C. and stirred for 16 h. The reaction was monitored by LCMS. After completion, the reaction mixture was concentrated under reduced pressure and the residue was purified by flash column chromatography in methanol and DCM. The required product was eluted in 4-8% of methanol and DCM. The fractions were concentrated to get compound 21 (105.00 mg; 0.12 mmol; 84.4%; off-white powder; purified product).

Yield: 105.00 mg (0.12 mmol), % Yield: 84.4

### Analytical Data:

LCMS: Column: ATLANTIS dC18 (50×4.6 mm) 5 μm; Mobile phase: A: 0.1% HCOOH in H₂O B: ACN; Flow Rate: 1.5 ml/min RT (min): 2.04; Purity: 96.44%; M+H: 840.10

### Step 2:

To a stirred solution of compound 21 (100.00 mg; 0.11 mmol; 1.00 eq.) in DCM (5.00 ml; 50.00 V) was added TFA (0.36 ml; 4.59 mmol; 40.00 eq.) at 0° C. The resulting solution was warm to RT and stirred for 3 h. The reaction was monitored by LCMS. After completion, the reaction mixture was concentrated under reduced pressure and the crude reaction mixture was purified by preparative HPLC using 0.1% HCOOH in H₂O and ACN. The product fractions were lyophilized to afford Example 11 as a formic acid salt (49.00 mg; 0.09 mmol; 77.9%; brown gum; purified product).

Yield: 49 mg (0.09 mmol), % Yield: 77.9

### Analytical Data:

LCMS: Column: XBridge C8, 3.5 μm, 4.6×50 mm; Mobile phase: A: 0.1% TFA in in H₂O B: ACN;

Flow Rate: 1.5 ml/min.

RT (min): 0. 1.88; Purity: 99.42%; M+H: 498.30

HPLC: Column: ATLANTIS dC18 (50×4.6 mm) 5 μm; Solvent A: water+0.1% TFA; Solvent B: ACN+0.1% TFA; Flow: 2 ml/min; Gradient: 0 min: 5% B, 8 min: 100% B, 8.1 min: 100% B, 8.5 min: 5% B, 10 min 5% B. RT (min): 4.09; Purity (max): 99.14%; Purity (220 nm): 99.53%.

¹HNMR (400 MHz, DMSO-d6): δ 8.44 (d, J=8.40 Hz, 1H), 8.16 (s, 1H), 7.51-7.67 (m, 5H), 7.34 (t, J=7.60 Hz, 1H), 4.75 (s, 4H), 3.52 (t, J=6.80 Hz, 3H), 3.38 (s, 2H), 3.21 (s, 2H), (s, 3H), 2.66 (s, 2H), 2.51 (d, J=2.00 Hz, 3H), 2.46 (d, J=36.00 Hz, 3H), 1.13-1.20 (m, 13H), (s, H).

### Chemical Synthesis of Example 12

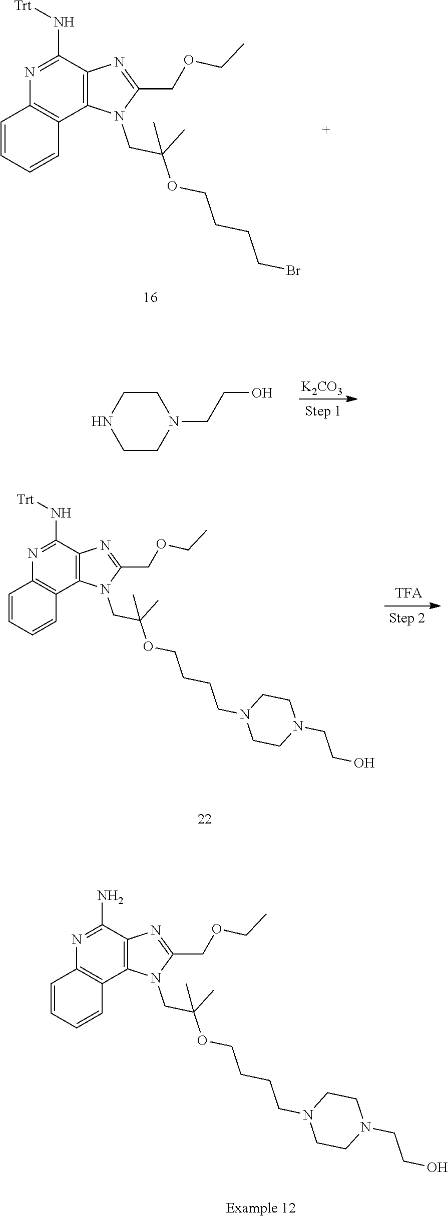

### Step 1:

To a stirred solution of compound 16 (60.00 mg; 0.08 mmol; 1.00 eq.) and 2-(piperazin-1-yl)ethan-1-ol (20.30 mg; 0.15 mmol; 2.00 eq.) in acetonitrile (1.80 ml; 30.00 V) was added potassium carbonate (32.33 mg; 0.23 mmol; 3.00 eq.) at RT. The suspension was heated to 80° C. and stirred for 16 h. The reaction was monitored by LCMS. After completion, the reaction mixture was partitioned between water and ethyl acetate, the separated organic layer was washed with brine solution, filtered and the filtrate was concentrated under reduced pressure to get a crude product of compound 22 (47.00 mg; 0.06 mmol; 80.3%; off-white powder; crude product).

Yield: 47 mg (0.06 mmol), % Yield: 80.3

### Analytical Data:

LCMS: Column: XBridge C8, 3.5 μm, 4.6×50 mm; Mobile phase: A: 0.1% TFA in in H₂O B: ACN; Flow Rate: 1.5 ml/min.

RT (min): 2.60; Purity: 96.73%; M+H: 741.30

### Step 2:

To a stirred solution of compound 22 (45.00 mg; 0.06 mmol; 1.00 eq.) in DCM (2.25 ml; 50.00 V) was added TFA (0.18 ml; 2.35 mmol; 40.00 eq.) at 0° C. The resulting solution was warm to RT and stirred for 3 h. The reaction was monitored by LCMS. After completion, the reaction mixture was concentrated under reduced pressure. The crude product was purified by preparative HPLC using 0.1% HCOOH in H₂O and ACN. The product fractions were lyophilized to afford Example 12 as a formic acid salt (19.00 mg; 0.03 mmol; 56.5%; off-white gum; purified product).

Yield: 19 mg (0.03 mmol), % Yield: 56.5

### Analytical Data:

LCMS: Column: ATLANTIS dC18 (50×4.6 mm) 5 μm; Mobile phase: A: 0.1% HCOOH in H₂O B: ACN; Flow Rate: 1.5 ml/min

RT (min): 0.98; Purity: 97.32%; M+H: 499.10

HPLC: Column: ATLANTIS dC18 (50×4.6 mm) 5 μm; Solvent A: water+0.1% TFA; Solvent B: ACN+0.1% TFA; Flow: 2 ml/min; Gradient: 0 min: 5% B, 8 min: 100% B, 8.1 min: 100% B, 8.5 min: 5% B, 10 min 5% B. RT (min): 9.14; Purity (max): 95.04%; Purity (220 nm): 92.88%.

¹HNMR (400 MHz, DMSO-d6): δ 8.54 (d, J=0.80 Hz, 2H), 7.76 (d, J=0.80 Hz, 1H), 7.65-7.74 (m, 1H), 7.45-7.49 (m, 1H), 3.77 (t, J=11.20 Hz, 2H), 3.65 (t, J=14.00 Hz, 3H), 2.81-2.89 (m, 6H), 2.69 (s, 4H), 2.36 (s, 2H), 2.00 (s, 3H), 1.15-1.33 (m, 14H), (s, H), (s, H), (s, H),

### Chemical Synthesis of Example 13

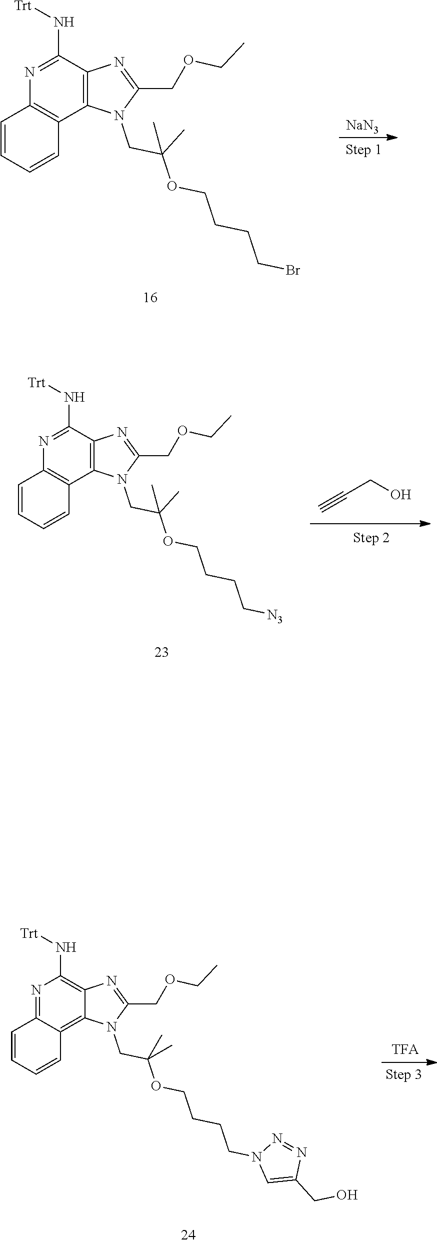

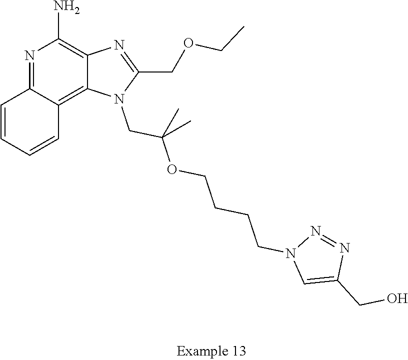 [SMILES: CCOCc1nc2c(N)nc3ccccc3c2n1CC(C)(C)OCCCCn1cc(CO)nn1]

### Step 1:

To a stirred solution of compound 16 (180.00 mg; 0.25 mmol; 1.00 eq.) and sodium azide (20.00 mg; 0.30 mmol; 1.20 eq.) in DMF (3.60 ml; 20.00 V). The suspension was stirred for 3 h at RT. The reaction was monitored by TLC. After completion, the reaction mixture was concentrated under reduced pressure. The residue was dissolved in water and extracted with DCM (8 mL). The organic layer was washed with water. The organic layer was dried over sodium sulphate, filtered and concentrated to get a crude product of compound 23 (150.00 mg; 0.15 mmol; 57.7%; off-white powder; crude product).

Yield: 150 mg (0.15 mmol), % Yield: 57.7

### Analytical Data:

LCMS: Column: XBridge C8, 3.5 μm, 4.6×50 mm; Mobile phase: A: 0.1% TFA in in H₂O; B: ACN;

Flow Rate: 1.5 ml/min

RT (min): 3.24; Purity: 63.86%; M+H: 654.30.

### Step 2:

To a solution of compound 23 (70.00 mg; 0.07 mmol; 1.00 eq.) and prop-2-yn-1-ol (3.91 mg; 0.07 mmol; 1.00 eq.) in THF (1.40 ml; 20.00 V) were added copper (I) iodide (7.90 mg; 0.04 mmol; 0.60 eq.) and ethyl-diisopropyl-amine (0.01 ml; 0.07 mmol; 1.00 eq.). The reaction mixture was stirred at RT for 3 h. The reaction was monitored by TLC. After completion, the reaction mixture was diluted with ethyl acetate and washed with water and brine solution. The organic layer was dried over sodium sulphate, filtered and concentrated to get a crude product.

This crude product was purified by column chromatography by using methanol/DCM as eluent desired product was eluted in 8% methanol in DCM. Required fractions were evaporated to get compound 24 (40.00 mg; 0.05 mmol; 70.6%; white solid; purified product).

Yield: 40 mg (0.05 mmol), % Yield: 70.6

### Analytical Data:

LCMS: Column: ATLANTIS dC18 (50×4.6 mm) 5 μm; Mobile phase: A: 0.1% HCOOH in H₂O B: ACN; Flow Rate: 1.5 ml/min.

RT (min): 2.23; Purity: 85.71%; M+H: 710.00

### Step 3:

To a stirred solution of compound 24 (40.00 mg; 0.05 mmol; 1.00 eq.) in DCM (2.00 ml; 50.00 V) was added TFA (0.30 ml; 3.87 mmol; 7.50 V) at 0° C. The resulting solution was warm to RT and stirred for 3 h. The reaction was monitored by TLC. After completion, the reaction mixture was concentrated under reduced pressure. The residue was purified by column chromatography by using methanol/DCM as eluent desired product was eluted in 15% methanol in DCM. Required fractions were evaporated to get Example 13 (18.00 mg; 0.04 mmol; 76.5%; light brown gum; purified product)

Yield: 18 mg (0.04 mmol), % Yield: 76.5.

### Analytical Data:

LCMS: Column: XBridge C8, 3.5 μm, 4.6×50 mm; Mobile phase: A: 0.1% TFA in in H₂O B: ACN;

Flow Rate: 1.5 ml/min

RT (min): 2.10; Purity: 97.88%; M+H: 468.20

HPLC: Column: Phenomenex Gemini C18 (150*4.6) mm, 3.0 μm; Mobile Phase A: 10 mM Ammonium acetate in milli-q water; Mobile phase B: Acetonitrile; Flow rate: 1.0 ml\min.

RT (min): 8.10; Purity (max): 95.26%; Purity (220 nm): 90.72%

### Chemical Synthesis of Example 14

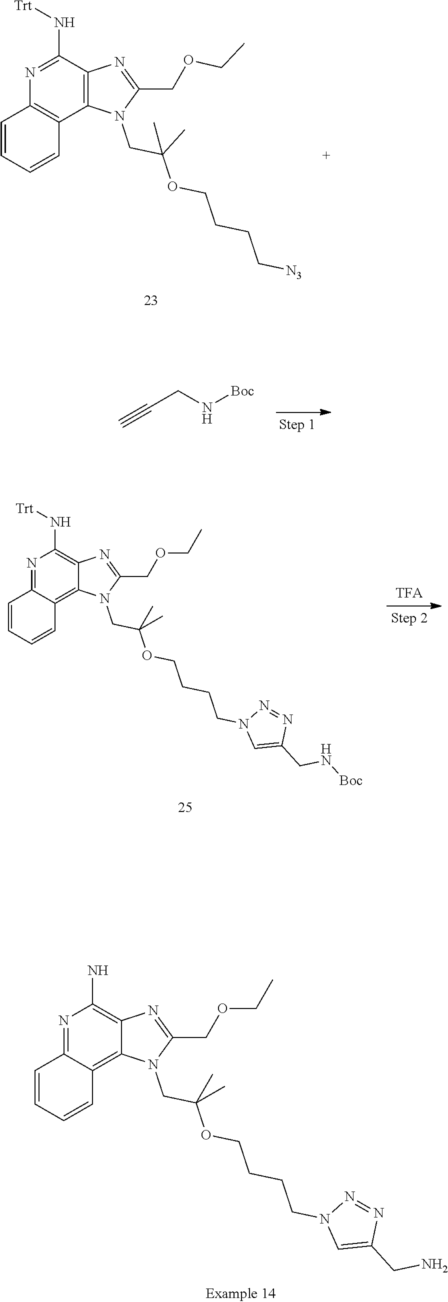

### Step 1:

To a solution of compound 23 (90.00 mg; 0.13 mmol; 1.00 eq.) and tert-butyl N-(prop-2-yn-1-yl) carbamate (20.99 mg; 0.13 mmol; 1.00 eq.) in THF (1.80 ml; 20.00 V) were added copper (I) iodide (15.30 mg; 0.08 mmol; 0.60 eq.) and ethyl diisopropyl amine (0.05 ml; 0.27 mmol; 2.00 eq.). The reaction mixture was stirred at RT for 3 h. The reaction was monitored by TLC.

After completion, the reaction mixture was diluted with ethyl acetate and washed with water and brine solution. Organic layer was dried over sodium sulphate, filtered and concentrated to get a crude compound. This crude product was purified by column chromatography by using methanol/DCM as eluent desired product was eluted in 8% methanol in DCM. Required fractions were evaporated to get compound 25 (50.00 mg; 0.06 mmol; 45.2%; white solid; purified product).

Yield: 50 mg (0.06 mmol), % Yield: 45.2.

### Analytical Data:

LCMS: Column: ATLANTIS dC18 (50×4.6 mm) 5 mm; Mobile Phase A: 0.1% HCOOH in water; B: 0.1% HCOOH in ACN; Flow Rate: 0.8 ml/min.

RT (min): 2.51; Purity: 96.95%; M+H: 809.00

### Step 2:

To a stirred solution of compound 25 (50.00 mg; 0.07 mmol; 1.00 eq.) in DCM (3.00 ml; 60.00 V) was added TFA (0.30 ml; 3.87 mmol; 6.00 V) at 0° C. The resulting solution was warm to RT and stirred for 3 h. The reaction was monitored by TLC. After completion, the reaction mixture was concentrated under reduced pressure to get a crude product. This crude product was purified by column chromatography by using methanol/DCM as eluent desired product was eluted in 15% methanol in DCM. Required fractions were evaporated to get Example 14 (15.00 mg; 0.03 mmol; 46.2%; off-white gum; purified product).

Yield: 15 mg (0.03 mmol), % Yield: 46.2

### Analytical Data:

LCMS: Column: XBridge C8, 3.5 μm, 4.6×50 mm; Mobile phase: A: 0.1% TFA in H₂O:ACN (95:5); Mobile phase B: 0.1% TFA in ACN; Flow Rate: 1.5 ml/min.

RT (min): 1.98; Purity: 99.94%; M+H: 467.20

HPLC: Column: ATLANTIS dC18 (50×4.6 mm) 5 mm; Mobile Phase A: 0.1% TFA in MilliQ water; Mobile Phase B: Acetonitrile; Flow Rate: 1.0 ml/min.

RT (min): 5.42; Purity (max): 99.86%; Purity (220 nm): 99.30%.

### Chemical Synthesis of Example 15

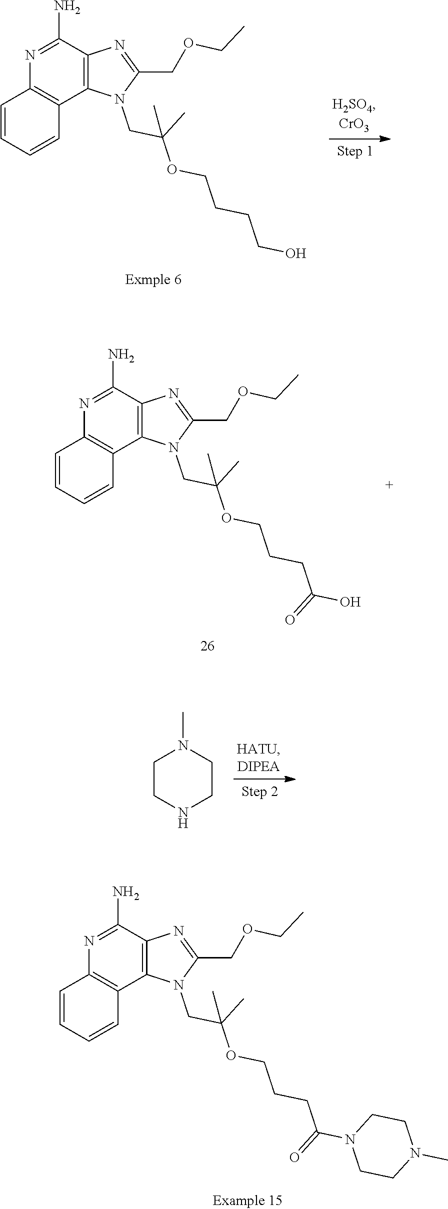

### Step 1:

To a stirred solution of Example 6 (5, 50.00 mg; 0.13 mmol; 1.00 eq.) in acetone (2.00 ml; 40.00 V) was added sulfuric acid and trioxochromium (0.10 ml) at 0° C. Resulting reaction mixture was stirred at RT for 1 h. After completion of the reaction, the reaction mixture was filtered, and the filtrate was concentrated under reduced pressure. The resulting crude was washed with diethyl ether (10 mL) to afford compound 26 (45.00 mg; 0.10 mmol; 81.1%; blue gum; crude product). The crude was directly used for the next step.

Yield: 45.00 mg (0.10 mmol), % Yield: 81.1.

### Analytical Data:

LCMS-Column: Atlantis dC18 (50×4.6 mm) 3.5 □m; Solvent A; 0.1% HCOOH in H₂O:ACN (95:5) Solvent B; ACN; Flow Rate: 1.5 mL/min.

RT (min): 1.47; Purity (max): 92.18%; M+H: 401.10.

### Step 2:

To a stirred solution of compound 26 (40.00 mg; 0.09 mmol; 1.00 eq.) in DMF (0.40 ml; 10.00 V) was added [Dimethylamino-([1,2,3]triazolo[4,5-b]pyridin-3-yloxy)-methylene]-dimethyl-ammonium; hexafluoro phosphate (67.06 mg; 0.17 mmol; 2.00 eq.). It was then added with ethyl-diisopropyl-amine (0.05 ml; 0.26 mmol; 3.00 eq.) and 1-methyl piperazine (0.02 ml; 0.17 mmol; 2.00 eq.) at 0° under nitrogen atmosphere. The reaction mixture was then stirred at RT for additional 2 h. The progress of the reaction was monitored by LCMS. After completion, the reaction mixture was lyophilized, and the crude product was purified by preparative HPLC. The product fractions were lyophilised to get Example 15 as a formic acid salt (13.00 mg; 0.02 mmol; 26.6%; brown gum; purified product).

Yield: 13.00 mg (0.02 mmol), % Yield: 26.6.

### Analytical Data:

LCMS-Column: Atlantis dC18 (50×4.6 mm) 3.5 μm; Solvent A; 0.1% HCOOH in H₂O:ACN (95:5) Solvent B; ACN; Flow Rate: 0.8 mL/min.

RT (min): 8.48; Purity (max): 94.47%; M+H: 483.20.

HPLC-Column: Atlantis dC18 (50×4.6 mm) 3.5 μm; Solvent A: 0.1% TFA in H₂O:ACN (95:5), Solvent B: ACN, Flow Rate: 0.8 mL/min.

RT (min): 5.21; Purity (max): 94.95%; Purity (220 nm): 92.87%.

¹H-NMR (400 MHz, CD3OD-d6): δ 8.83 (d, J=8.00 Hz, 1H), 7.72 (d, J=8.40 Hz, 1H), 7.58 (t, J=7.60 Hz, 1H), 7.41 (t, J=7.20 Hz, 1H), 4.88 (s, 4H), 3.65 (t, J=6.80 Hz, 2H), 3.61 (s, 2H), 3.50 (d, J=3.20 Hz, 2H), 3.15 (d, J=4.00 Hz, 2H), 2.34 (s, 6H), 1.91 (t, J=6.80 Hz, 2H), 1.58 (q, J=6.00 Hz, 2H), 1.27 (q, J=7.20 Hz, 11H).

### Chemical Synthesis of Example 16

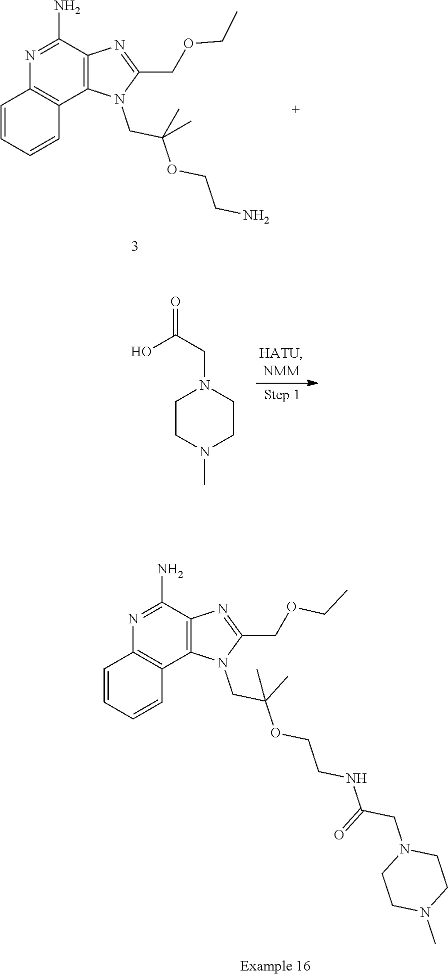 [SMILES: CCOCc1nc2c(N)nc3ccccc3c2n1CC(C)(C)OCCN ; CN1CCN(CC(=O)O)CC1 ; CCOCc1nc2c(N)nc3ccccc3c2n1CC(C)(C)OCCNC(=O)CN1CCN(C)CC1]

### Step 1:

(4-Methyl-piperazin-1-yl) acetic acid (10,000 mg; 1.00 eq.) was dissolved in DMF (1,000 ml) and cooled to 0° C. Then 4-Methylmorpholine (17,374 μl; 2.50 eq.), [Dimethylamino-([1,2,3]triazolo[4,5-b]pyridin-3-yloxy)-methylene]-dimethyl-ammonium; hexafluoro phosphate (26,439 mg; 1.10 eq.) and finally compound 3 (23,222 mg; 1.00 eq.) were added. The reaction mixture was allowed to warm up to room temperature and was stirred for 1 h. The reaction solution was concentrated under reduced pressure and the residue was purified by preparative HPLC eluted with water/acetonitrile (0.1% TFA). The product containing fractions were combined and lyophilized overnight to afford Example 16 as a trifluoroacetic acid salt (31.00 mg; 0,050 mmol; 79%) as a transparent solid.

### Analytical Data:

HPLC-MS: column: Chromolith HR C18 5.0 μm; 50-4.6 mm; Method Info (Chromolith): A: H₂O+0.05% HCOOH|B: MeCN+0.04% HCOOH|T: 40° C.|Flow: 3.3 ml/min|MS: 100-2000 amu positive|1%->100% B: 0->2.0 min|100% B: 2.0->2.5 min

RT (min): 0.98; Purity (max): 98.3%; M+H: 498.6.

### Chemical Synthesis of Example 17

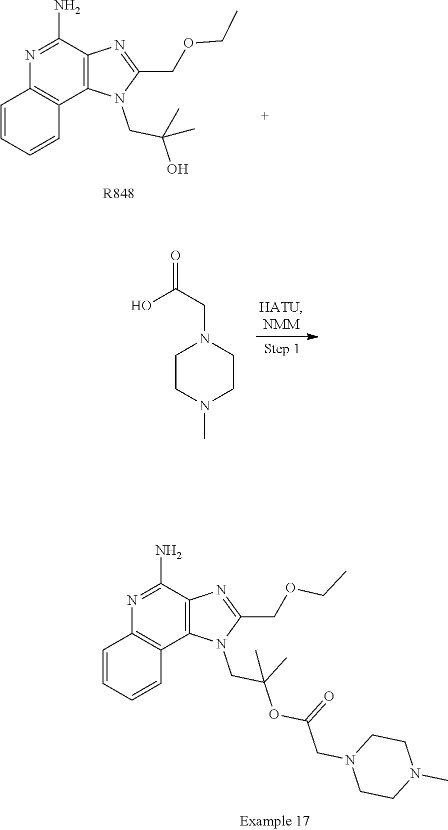

### Step 1:

R848 (20.00 mg; 0,062 mmol; 1.0 eq.), 2-(4-methylpiperazin-1-yl) acetic acid (29,588 mg; 0,187 mmol; 3.0 eq.) and 4-(pyrrolidin-1-yl)pyridine (13,860 mg; 0,094 mmol; 1.5 eq.) were combined with dichloromethane (2,000 ml) to give a white suspension. N,N′-Diisopropylcarbodiimide (0,029 ml; 0,187 mmol; 3.0 eq.) was added. The reaction mixture was heated at 50° C. overnight. After completion, the solution was directly injected onto a preparative HPLC eluted with water/acetonitrile (0.1% TFA). The fractions containing the product were joined together and lyophilized overnight to afford Example 17 as a trifluoroacetic acid salt (8.5 mg; 22.5%, transparent oil, purified product)

### Analytical Data:

HPLC-MS: column: Chromolith HR C18 5.0 μm; 50-4.6 mm; Method Info (Chromolith): A: H2O+0.05% HCOOH|B: MeCN+0.04% HCOOH|T: 40° C.|Flow: 3.3 ml/min|MS: 100-2000 amu positive|1%->100% B: 0->2.0 min|100% B: 2.0->2.5 min

RT (min): 0.97; Purity (max): 94.0%; M+H=455

### Chemical Synthesis of Example 18

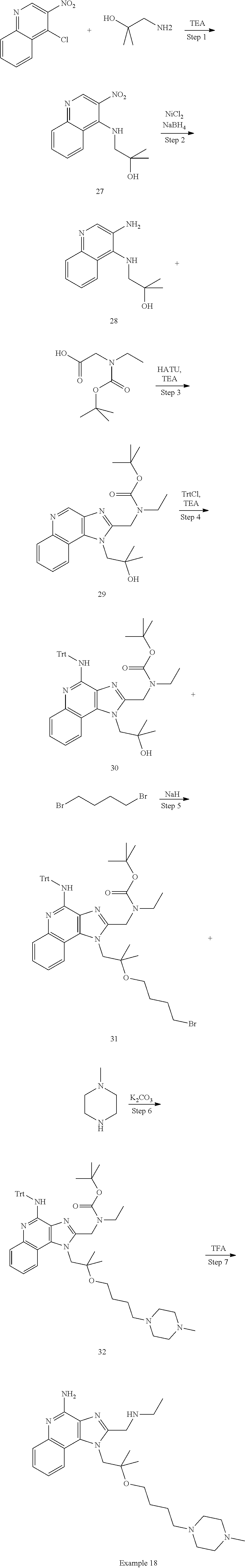

### Step 1:

To solution of 4-chloro-3-nitroquinoline (4.50 g; 21.14 mmol; 1.00 eq.) dissolved in DCM (45.00 ml; 10.00 V) were added triethyl amine (9.00 ml; 63.42 mmol; 3.00 eq.) followed by 1-amino-2-methylpropan-2-ol (3.81 g; 42.28 mmol; 2.00 eq.) at RT. The reaction mixture was refluxed for 3 h. The reaction monitored by TLC. After completion, the reaction mixture was concentrated under vacuum and water was added, the solid was formed and filtered under vacuum to obtain compound 27 (5.30 g; 19.93 mmol; 94.3%; yellow solid; purified product).

Yield: 5.30 g (19.93 mmol), % Yield: 94.3.

### Analytical Data:

LCMS-Column: Atlantis dC18 (50×4.6 mm) 3.5 μm; Solvent A; 0.1% HCOOH in H₂O:ACN (95:5) Solvent B; ACN; Flow Rate: 0.8 mL/min.

RT (min): 0.436; Purity (max): 98.27%; M+H: 260.20.

### Step 2:

To a solution of compound 27 (5.30 g; 20.28 mmol; 1.00 eq.) in a solvent of water (4.58 ml; 0.86 V) and methanol (50.00 ml; 9.43 V) were added nickel (II) chloride hexahydrate (0.19 g; 0.80 mmol; 0.04 eq.) followed by sodium borohydride (1.57 g; 40.67 mmol; 2.00 eq.) at 0° C.

The reaction mixture was stirred at RT for 2 h. The reaction was monitored by TLC. After completion, the black solution was filtered through celite and the filtrate was concentrated to get a crude product. Then the crude product was purified by flash column chromatography (3% MeOH in DCM) to obtain compound 28 (4.20 g; 16.55 mmol; 81.6%; orange powder; purified product).

Yield: 4.20 g (16.55 mmol), % Yield: 81.6.

### Analytical Data:

LCMS-Column: Atlantis dC18 (50×4.6 mm) 3.5 μm; Solvent A; 0.1% HCOOH in H₂O:ACN (95:5) Solvent B; ACN; Flow Rate: 0.8 mL/min.

RT (min): 0.37; Purity (max): 91.11%; M+H: 232.20.

### Step 3:

To a stirred solution of compound 28 (4.20 g; 18.16 mmol; 1.00 eq.) in DMF (42.00 ml; 10.00 V) were added 2-{[(tert-butoxy)carbonyl](ethyl)amino}acetic acid (5.59 g; 27.24 mmol; 1.50 eq.), [Dimethylamino-([1,2,3]triazolo[4,5-b]pyridin-3-yloxy)-methylene]-dimethyl-ammonium; hexafluoro phosphate (20.92 g; 54.48 mmol; 3.00 eq.), triethyl amine (7.14 ml; 54.48 mmol; 3.00 eq.) and 4-Dimethylaminopyridine (0.22 g; 1.82 mmol; 0.10 eq.). The reaction mixture was stirred at RT for 3 h. The reaction was monitored by LCMS. After completion, the reaction mixture was concentrated under vacuum to get a residue. The residue was diluted with DCM and washed with water, the organic layer was collected and concentrated under vacuum to get an intermediate, which was dissolved in ethanol (84.00 ml; 20.00 V) and sodium hydroxide (1.02 g; 2.54 mmol; 0.14 eq.) in water (2.10 ml; 0.50 V) was added at RT. The cyclisation reaction was refluxed for 6 h and the reaction was monitored by LCMS. After completion, the reaction mixture was concentrated under vacuum to get a crude product. The crude product was purified by silica gel chromatography using 7% MeOH in DCM to afford as compound 29 (4.90 g; 9.27 mmol; 51.0%; brown light solid; purified product).

Yield: 4.90 g (9.27 mmol), % Yield: 51.0.

### Analytical Data:

LCMS-Column: Atlantis dC18 (50×4.6 mm) 3.5 μm; Solvent A; 0.1% HCOOH in H₂O:ACN (95:5) Solvent B; ACN; Flow Rate: 0.8 mL/min.

RT (min): 0.67; Purity (max): 82.73%; M+H: 399.20.

### Step 4:

To a stirred solution of compound 29 (0.80 g; 1.92 mmol; 1.00 eq.) in Acetonitrile (24.00 ml; 30.00 V) was added to triethyl amine (1.36 ml; 9.58 mmol; 5.00 eq.) followed by [chloro(diphenyl)methyl]benzene (1.08 g; 3.83 mmol; 2.00 eq.) under nitrogen atmosphere. The resultant suspension was heated to 100° C. and stirred for 16 h. The reaction was monitored by TLC. After completion, the solvent was removed to get a crude product. The crude product was purified by flash column chromatography in 60-120 silicagel with ethylacetate and pet ether. The product was eluted in 40-100% of Ethylacetate and Pet ether and eluents were concentrated to afford compound 30 (800.00 mg; 1.22 mmol; 63.6%; white solid; purified product).

Yield: 800.00 mg (1.22 mmol), % Yield: 63.6.

### Analytical Data:

LCMS-Column: Atlantis dC18 (50×4.6 mm) 3.5 μm; Solvent A; 0.1% HCOOH in H₂O:ACN (95:5) Solvent B; ACN; Flow Rate: 0.8 mL/min.

RT (min): 1.14; Purity (max): 93.25%; M+H: 656.20.

### Step 5:

To a stirred solution of NaH (60%) (0.05 g; 1.19 mmol; 1.50 eq.) in DMSO (5.60 ml; 10.00 V) at 0° C. under nitrogen atmosphere was added dropwise compound 30 (0.56 g; 0.80 mmol; 1.00 eq.) in DMSO (5.60 ml; 10.00 V) and reaction mixture was stirred at RT for 30 min. Then 1,4-dibromobutane (0.53 g; 2.39 mmol; 3.00 eq.) was added at 0° C. and the reaction mixture was allow to stir at RT for 1 h under nitrogen atmosphere. The reaction was monitored by TLC. After completion, the reaction mixture was quenched with ice water at 0° C. The reaction mixture was diluted with ethyl acetate and organic layer was separated, dried with sodium sulphate, filtered, and concentrated under vacuum. The crude product was purified by silica gel chromatography and product was eluted with 15% ethyl acetate in hexane. The required fractions were evaporated to get compound 31 (23.00 mg; 0.03 mmol; 3.6%; white gum; purified product).

Yield: 23.00 mg (0.03 mmol), % Yield: 3.6.

### Analytical Data:

HPLC-Column: Atlantis dC18 (50×4.6 mm) 3.5 μm; Solvent A: 0.1% TFA in H₂O:ACN (95:5), Solvent B: ACN, Flow Rate: 0.8 mL/min.

RT (min): 3.09; Purity (max): 96.95%; M+H: 790.30.

### Step 6:

To a stirred solution of compound 31 (80.00 mg; 0.08 mmol; 1.00 eq.) and 1-methyl piperazine (0.01 ml; 0.09 mmol; 1.10 eq.) in acetonitrile (2.40 ml; 30.00 V) was added potassium carbonate (23.91 mg; 0.17 mmol; 2.00 eq.) at RT. The suspension was heated to 50° C. and stirred for 16 h. The reaction was monitored by LCMS. After completion, the solvent was removed to get a crude product. The crude product was dissolved in water and extracted with DCM (8 mL). The organic layer was washed with water. The organic layer was dried over sodium sulphate, filtered, and concentrated to obtain compound 32 (55.00 mg; 0.06 mmol; 67.4%; off-white gum; purified product).

Yield: 55.00 mg (0.06 mmol), % Yield: 67.4.

### Analytical Data:

LCMS-Column: Atlantis dC18 (50×4.6 mm) 3.5 μm; Solvent A; 0.1% HCOOH in H₂O:ACN (95:5) Solvent B; ACN; Flow Rate: 0.8 mL/min.

RT (min): 1.70; Purity (max): 84.18%; M+H: 810.40.

### Step 7:

To a stirred solution of compound 32 (55.00 mg; 0.04 mmol; 1.00 eq.) in DCM (2.75 ml; 50.00 V) was added TFA (0.13 ml; 1.70 mmol; 40.00 eq.) at 0° C. The resulting solution was warm to RT and stirred for 3 h. The reaction was monitored by LCMS. After completion, the solvent was removed under reduced pressure at below 35° C. to get a crude product. The crude product was purified by preparative HPLC in TFA method and product fraction was lyophilized to afford Example 18 as a trifluoroacetic acid salt (13.10 mg; 0.02 mmol; 51.6%; white gum; purified product).

Yield: 13.10 mg (0.02 mmol), % Yield: 51.6.

### Analytical Data:

HPLC-Column: Atlantis dC18 (50×4.6 mm) 3.5 μm; Solvent A: 0.1% TFA in H₂O:ACN (95:5), Solvent B: ACN, Flow Rate: 0.8 mL/min.

RT (min): 8.32; Purity (max): 97.35%; M+H: 468.30.

¹H-NMR (400 MHz, DMSO-d6): δ 9.23 (s, br, 2H), 8.99 (s, br, 2H), 8.61 (d, J=8.00 Hz, 1H), 7.83 (d, J=8.00 Hz, 1H), 7.75 (t, J=15.20 Hz, 1H), 7.55 (t, J=15.20 Hz, 1H), 4.68 (s, br, 6H), 3.19-2.22 (m, 15H), 1.20-1.31 (m, 13H).

### Chemical Synthesis of Example 19

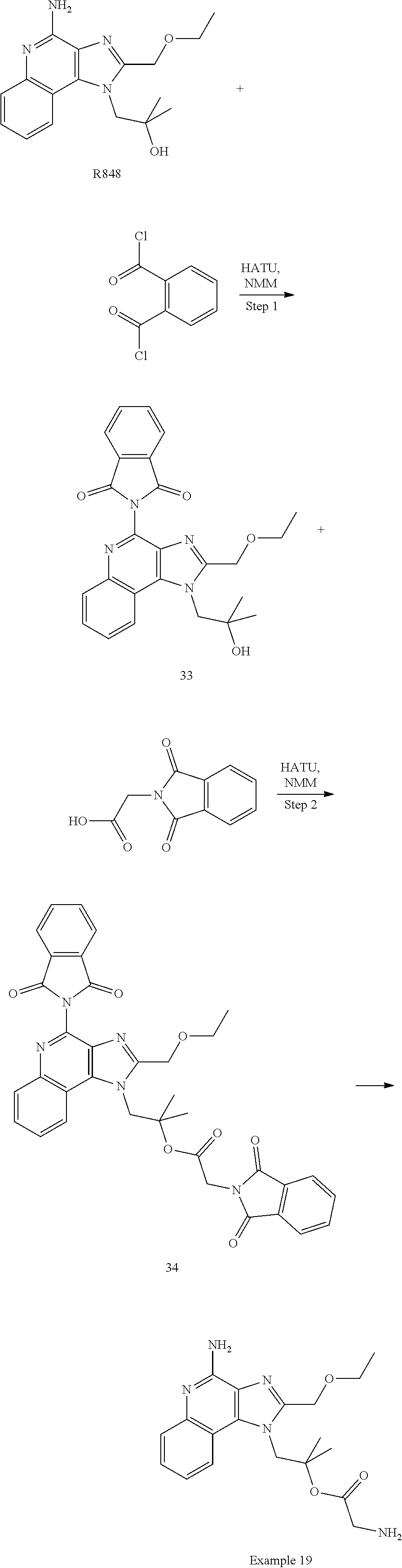

### Step 1:

R848 (18.00 mg; 0.057 mmol; 1.0 eq.) was added to toluene (2.00 ml) to give a white suspension. 1,4-Diazabicyclo[2.2.2]octane (0.017 ml; 0.170 mmol; 3.0 eq.) and phthaloyl dichloride (0.010 ml; 0.068 mmol; 1.2 eq.) were added. The reaction mixture was stirred at 110° C. for 2 h and monitored by LCMS. After completion, the mixture was cooled and then diluted with ethyl acetate and washed with 1N HCl. The organic phase was separated and the water layer was extracted with ethyl acetate. The organic layers were combined, dried over magnesium sulphate, filtered, and concentrated in vacuo to give a crude product of compound 33 (25.20 mg, 0.070 mmol, white gum; crude product), which was used without further purification in the next reaction.

### Analytical Data:

HPLC-MS: column: Chromolith HR C18 5.0 μm; 50-4.6 mm; Method Info (Chromolith): A: H₂O+0.05% HCOOH|B: MeCN+0.04% HCOOH|T: 40° C.|Flow: 3.3 ml/min|MS: 100-2000 amu positive|1%->100% B: 0->2.0 min|100% B: 2.0->2.5 min

RT (min): 1.50; Purity (max): 30.81%; M+H=445

### Step 2:

Compound 33 obtained from the step 1 as a crude material (18.00 mg; 0.040 mmol; 1.0 eq.), (1,3-Dioxo-1,3-dihydro-isoindol-2-yl)-acetic acid (24.93 mg; 0.121 mmol; 3.0 eq.) and 4-(pyrrolidin-1-yl)pyridine (9.00 mg; 0.061 mmol; 1.5 eq.) were combined with DCM (2.00 ml) to give a white suspension. N,N′-diisopropylcarbodiimide (0.019 ml; 0.121 mmol; 3.0 eq.) and molecular sieves were added. The reaction mixture was heated to 50° C. overnight and monitored by LCMS. After completion, the reaction mixture was purified by a preparative RP-HPLC eluted with water/acetonitrile (0.1% TFA). The fractions containing the product were joined together and lyophilized overnight to afford compound 34 (16.70 mg, 0.02 mmol, 61.0%, white powder, purified product).

Yield: 16.70 mg (0.02 mmol), % Yield: 61.0.

### Analytical Data:

HPLC-MS: column: Chromolith HR C18 5.0 μm; 50-4.6 mm; Method Info (Chromolith): A: H₂O+0.05% HCOOH|B: MeCN+0.04% HCOOH|T: 40° C.|Flow: 3.3 ml/min|MS: 100-2000 amu positive|1%->100% B: 0->2.0 min|100% B: 2.0->2.5 min

RT (min): 1.725; Purity (max): 93.5%; M+H=631.7.

### Step 3:

Compound 34 (16.700 mg; 0.026 mmol; 1.0 eq.) was combined with THF (2.00 ml) to give a white suspension. To this suspension was added hydrazine solution in water (35%) (0.057 ml; 0.624 mmol; 23.6 eq.) and the solution was stirred at room temperature for 4 h and monitored by LCMS. After completion, the reaction mixture was directly injected onto a preparative HPLC eluted with water/acetonitrile (0.1% TFA). The fractions containing the product were joined together and lyophilized overnight to Example 19 as a trifluoroacetic acid salt (3.70 mg; 0.01 mmol; 29.0%; white powder; purified product).

Yield: 3.70 mg (0.01 mmol), % Yield: 29.0.

### Analytical Data:

HPLC-MS: column: Chromolith HR C18 5.0 μm; 50-4.6 mm; Method Info (Chromolith): A: H₂O+0.05% HCOOH|B: MeCN+0.04% HCOOH|T: 40° C.|Flow: 3.3 ml/min|MS: 100-2000 amu positive|1%->100% B: 0->2.0 min|100% B: 2.0->2.5 min

RT (min): 0.902; Purity (max): 100%; M+H=371.9.

### Chemical Synthesis of Example 20

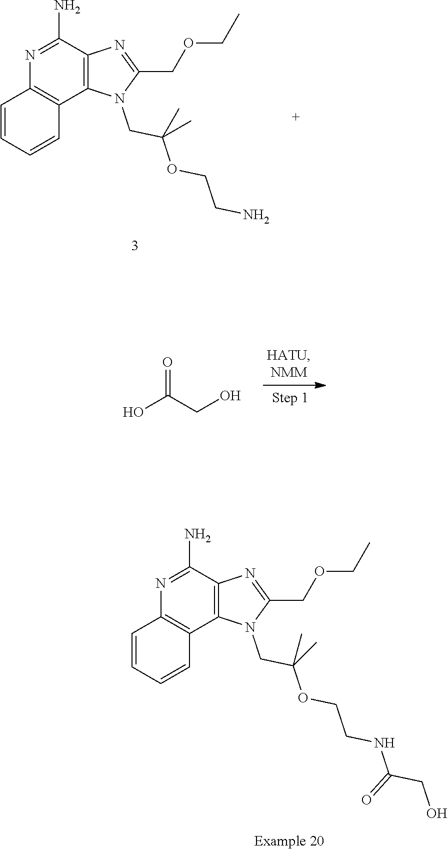 [SMILES: CCOCc1nc2c(N)nc3ccccc3c2n1CC(C)(C)OCCN ; O=C(O)CO ; CCOCc1nc2c(N)nc3ccccc3c2n1CC(C)(C)OCCNC(=O)CO]

Glycolic acid (6.482 mg; 1.00 eq.) was dissolved in DMF (2.00 ml), then 4-methylmorpholine (0.023 ml; 2.50 eq.) and [Dimethylamino-([1,2,3]triazolo[4,5-b]pyridin-3-yloxy)-methylene]-dimethyl-ammonium; hexafluoro phosphate (35.294 mg; 1.10 eq.) were added and stirred for 10 minutes. Finally was added Compound 3 (31.00 mg; 1.00 eq.) and the mixture was stirred at room temperature overnight and monitored by LCMS. After overnight reaction the starting material was not completely converted into the product, thus, to the mixture was added a solution of glycolic acid (6.482 mg; 1.00 eq.), [Dimethylamino-([1,2,3]triazolo[4,5-b]pyridin-3-yloxy)-methylene]-dimethyl-ammonium; hexafluoro phosphate (35,294 mg; 1.10 eq.) and 4-Methylmorpholine (0.023 ml; 2.50 eq.) in DMF (0.500 ml) and it was stirred overnight again and monitored by LCMS. After completion, the reaction solution was concentrated under reduced pressure and the residue was purified by preparative HPLC eluted with water/acetonitrile (0.1% TFA). The product containing fractions were combined and lyophilized overnight to afford Example 20 (17.10 mg; 0.041 mmol; 48.8%; off-white solid; purified product).

Yield: 17.10 mg (0.041 mmol), % Yield: 48.8.

### Analytical Data:

HPLC-MS: column: Chromolith HR C18 5.0 μm; 50-4.6 mm; Method Info (Chromolith): A: H₂O+0.05% HCOOH|B: MeCN+0.04% HCOOH|T: 40° C.|Flow: 3.3 ml/min|MS: 100-2000 amu positive|1%->100% B: 0->2.0 min|100% B: 2.0->2.5 min

RT (min): 1.10; Purity (max): 99.9%; M+H=416.1.

### Chemical Synthesis of Example 21

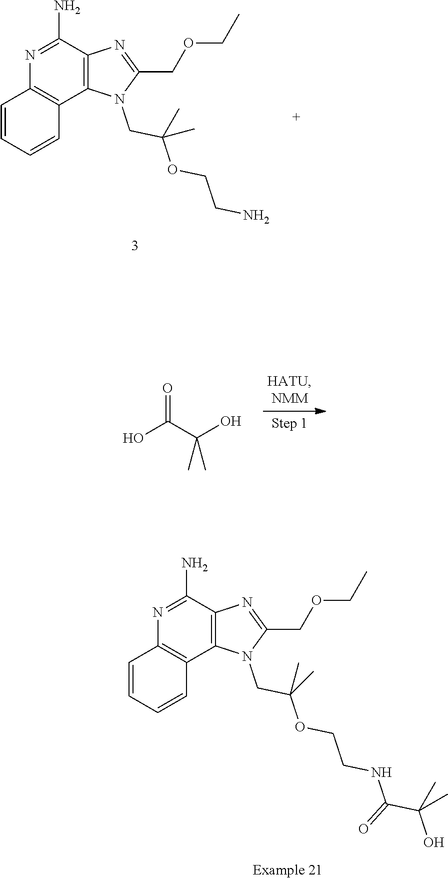

2-hydroxy isobutyric acid (8.675 mg; 1.00 eq.) was dissolved in DMF (2.000 ml), then 4-methylmorpholine (0.018 ml; 2.00 eq.) and [Dimethylamino-([1,2,3]triazolo[4,5-b]pyridin-3-yloxy)-methylene]-dimethyl-ammonium; hexafluoro phosphate (31.050 mg; 1.00 eq.) were added and stirred for 10 minutes. Finally was added compound 3 (30.000 mg; 1.00 eq.) and the mixture was stirred at room temperature overnight and monitored by LCMS. The reaction solution was concentrated under reduced pressure and the residue was purified by preparative HPLC eluted with water/acetonitrile (0.1% TFA). The product containing fractions were combined and lyophillized overnight to afford Example 21 as a trifluoroacetic acid salt (29.000 mg; 0.052 mmol; 63.7%; white solid, purified product).

Yield: 29.000 mg (0.052 mmol), % Yield: 63.7.

### Analytical Data:

HPLC-MS: column: Chromolith HR C18 5.0 μm; 50-4.6 mm; Method Info (Chromolith): A: H₂O+0.05% HCOOH|B: MeCN+0.04% HCOOH|T: 40° C.|Flow: 3.3 ml/min|MS: 100-2000 amu positive|1%->100% B: 0->2.0 min|100% B: 2.0->2.5 min

RT (min): 1.103; Purity (max): 100%; M+H=444.0.

### Chemical Synthesis of Example 22

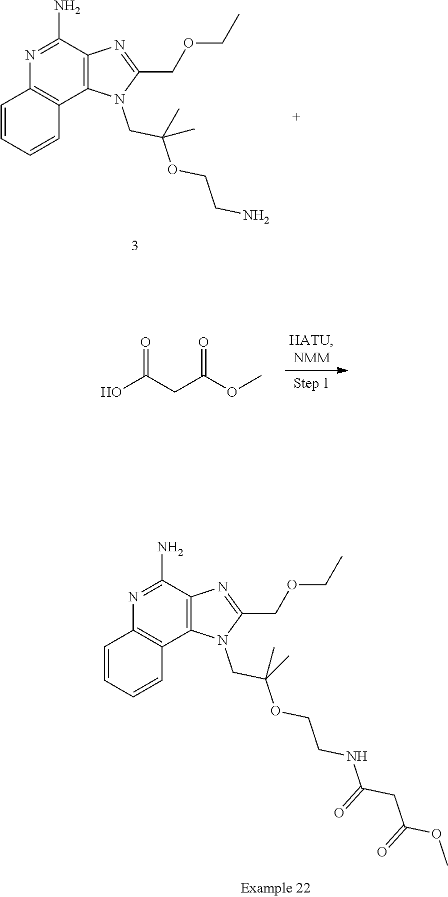

Malonic acid monomethyl ester (14.248 μl; 1.00 eq.) was dissolved in DMF (2,000 ml), then 4-methylmorpholine (0.030 ml; 2.00 eq.) and [Dimethylamino-([1,2,3]triazolo[4,5-b]pyridin-3-yloxy)-methylene]-dimethyl-ammonium; hexafluoro phosphate (51.750 mg; 1.00 eq.) were added and stirred for 10 minutes. Finally was added compound 3 (50.000 mg; 1.00 eq.) and the mixture was stirred at room temperature overnight and monitored by LCMS. The reaction solution was concentrated under reduced pressure and the residue was purified by preparative HPLC eluted with water/acetonitrile (0.1% TFA). The product containing fractions were combined and lyophilized overnight to afford Example 22 as a trifluoroacetic acid salt (52.300 mg; 0.084 mmol; 61.9%; transparent glass solid; purified product).

Yield: 52.300 mg (0.084 mmol), % Yield: 61.9.

### Analytical Data:

HPLC-MS: column: Chromolith HR C18 5.0 μm; 50-4.6 mm; Method Info (Chromolith): A: H₂O+0.05% HCOOH|B: MeCN+0.04% HCOOH|T: 40° C.|Flow: 3.3 ml/min|MS: 100-2000 amu positive|1%->100% B: 0->2.0 min|100% B: 2.0->2.5 min

RT (min): 1.11; Purity (max): 92.0%; M+H=458.0.

### Chemical Synthesis of Example 23

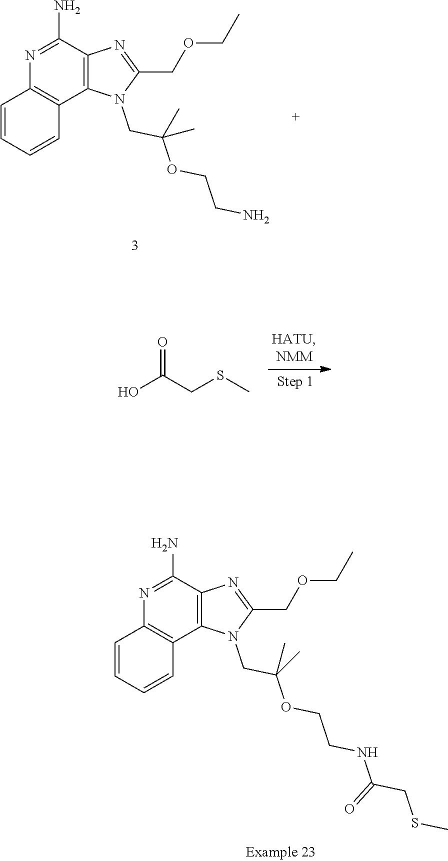

Mehtyl thio acetic acid (8.203 μl; 1.00 eq.) was dissolved in DMF (2.000 ml). N-ethyl diisopropyl amine (79.306 μl; 5.00 eq.) and [Dimethylamino-([1,2,3]triazolo-[4,5-b]pyridin-3-yloxy)-methylene]-dimethyl-ammonium; hexafluoro phosphate (70.927 mg; 2.00 eq.) were added and stirred for 20 min followed by addition of compound 3 (33.339 mg; 1.00 eq.) was added and the reaction mixture was stirred at room temperature for 2 h and monitored by LCMS. After completion, the reaction mixture was purified by prep. HPLC over a Sunfire column eluted with water/acetonitrile (0.1% TFA). The fractions containing product were combined, concentrated and lyophilized to obtain Example 23 as a trifluoroacetic acid salt (16.500 mg; 0.028 mmol; 30.3%; beige solid; purified product).

Yield: 16.500 mg (0.028 mmol), % Yield: 30.3.

### Analytical Data:

HPLC-MS: column: Chromolith HR C18 5.0 μm; 50-4.6 mm; Method Info (Chromolith): A: H₂O+0.05% HCOOH|B: MeCN+0.04% HCOOH|T: 40° C.|Flow: 3.3 ml/min|MS: 100-2000 amu positive|1%->100% B: 0->2.0 min|100% B: 2.0->2.5 min

RT (min): 1.177; Purity (max): 95.9%; M+H=446.2.

### Chemical Synthesis of Example 24

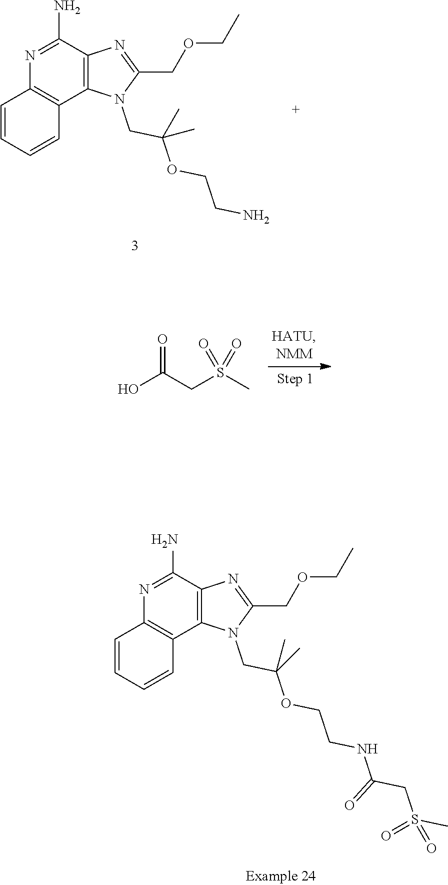

Methane sulfonyl acetic acid (12.058 mg; 0.087 mmol; 1.2 eq.) was dissolved in DMF (2.000 ml). 4-methyl morpholine (15.994 μl; 0.145 mmol; 2.0 eq.) and [Dimethylamino-([1,2,3]triazolo[4,5-b]pyridin-3-yloxy)-methylene]-dimethyl-ammonium; hexafluoro phosphate (33.188 mg; 0.087 mmol; 1.2 eq.) were added and stirred for 10 min followed by addition of compound 3 (26.000 mg; 0.073 mmol; 1.0 eq.). The reaction mixture was stirred overnight and monitored by LCMS. After completion, the reaction mixture was purified by a preparative HPLC eluted with water/acetonitrile (0.1% TFA). The fractions containing the product were collected and lyophilized overnight to obtain Example 24 as a trifluoroacetic acid salt (9.10 mg; 0.01 mmol; 20.5%; light beige solid; purified product).

Yield: 9.10 mg (0.01 mmol), % Yield: 20.5.

### Analytical Data:

HPLC-MS: column: Chromolith HR C18 5.0 μm; 50-4.6 mm; Method Info (Chromolith): A: H₂O+0.05% HCOOH|B: MeCN+0.04% HCOOH|T: 40° C.|Flow: 3.3 ml/min|MS: 100-2000 amu positive|1%->100% B: 0->2.0 min|100% B: 2.0->2.5 min

RT (min): 1.14; Purity (max): 97.0%; M+H=478.1.

### Chemical Synthesis of Example 25

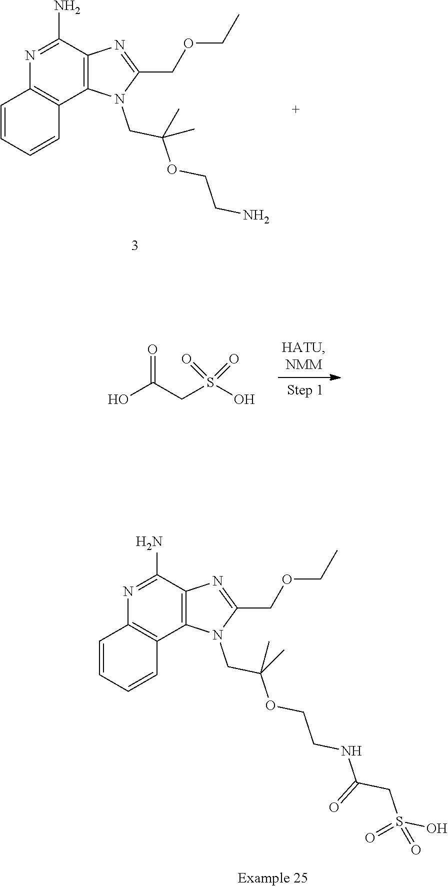 [SMILES: CCOCc1nc2c(N)nc3ccccc3c2n1CC(C)(C)OCCN ; O=C(O)CS(=O)(=O)O ; CCOCc1nc2c(N)nc3ccccc3c2n1CC(C)(C)OCCNC(=O)CS(=O)(=O)O]

2-Sulfoacetic acid (12.740 mg; 0.087 mmol; 1.2 eq.) was dissolved in DMF (2,000 ml). 4-methylmorpholine (15.994 μl; 0.145 mmol; 2.0 eq.) and [Dimethylamino-([1,2,3]triazolo[4,5-b]pyridin-3-yloxy)-methylene]-dimethyl-ammonium; hexafluoro phosphate (33.188 mg; 0.087 mmol; 1.2 eq.) were added and stirred for 10 min followed by addition of compound 3 (26.000 mg; 0.073 mmol; 1.0 eq.). The reaction mixture was stirred at room temperature overnight and monitored by LCMS. After completion, the reaction mixture was purified by a preparative HPLC eluted with water/acetonitrile (0.1% TFA). The fractions containing the product were collected and lyophilized overnight to obtain Example 25 as a trifluoroacetic acid salt (26.3 mg; 0.04 mmol; 60.3%; white powder; purified product).

Yield: 26.3 mg (0.04 mmol), % Yield: 60.3.

### Analytical Data:

HPLC-MS: column: Chromolith HR C18 5.0 μm; 50-4.6 mm; Method Info (Chromolith): A: H₂O+0.05% HCOOH|B: MeCN+0.04% HCOOH|T: 40° C.|Flow: 3.3 ml/min|MS: 100-2000 amu positive|1%->100% B: 0->2.0 min|100% B: 2.0->2.5 min

RT (min): 1.108; Purity (max): 99%; M+H=480.1.

### Chemical Synthesis of Example 26

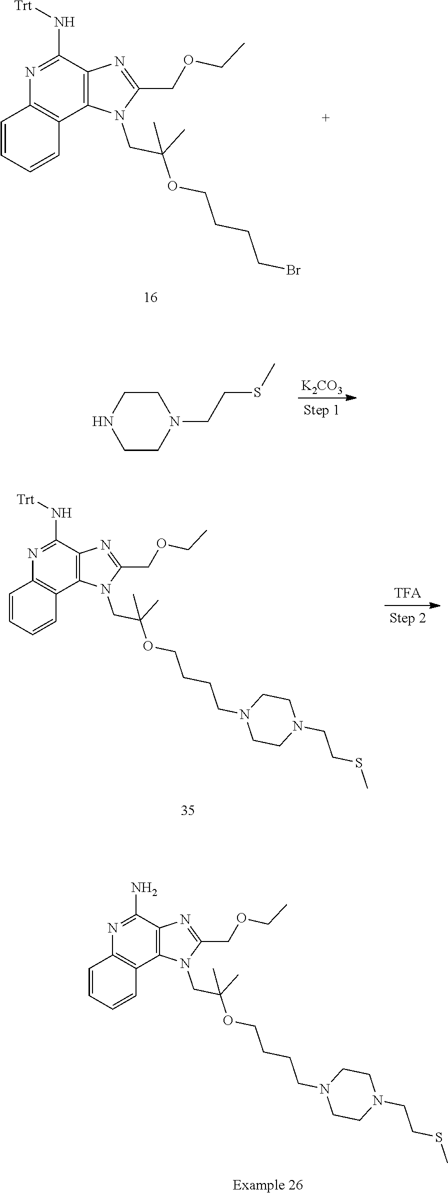

### Step 1:

To a solution of compound 16 (200.00 mg; 0.29 mmol; 1.00 eq.) and 1-[2-(methylsulfanyl)ethyl]piperazine (69.52 mg; 0.43 mmol; 1.50 eq.) in acetionitrile (4.000 ml), potassium carbonate (122.33 mg; 0.87 mmol; 3.00 eq.) was added and stirred at 60° C. overnight. The reaction was monitored by TLC. After completion of the reaction, the reaction mixture was concentrated and purified the crude residue by silica gel chromatography using 10% methanol: DCM to afford compound 35 (152.73 mg; 0.19 mmol; 67.3%; Purified Product).

Yield: 152.73 mg (0.19 mmol), % Yield: 67.3.

### Analytical Data:

HPLC: Column: XBridge C8, 3.5 μm, 4.6×50 mm; Solvent A: water+0.1% TFA; Solvent B: ACN+0.1% TFA; Flow: 2 ml/min; Gradient: 0 min: 5% B, 8 min: 100% B, 8.1 min: 100% B, 8.5 min: 5% B, 10 min 5% B.

RT (min): 2.59; Purity (max): 98.2%; M+H=771.40.

### Step 2:

To a solution of compound 35 (175.00 mg; 0.23 mmol; 1.00 eq.) in DCM (1.75 ml), trifluoro-acetic acid (0.18 ml; 2.27 mmol; 10.00 eq.) was added dropwise and stirred overnight at RT. The reaction was monitored by TLC. After completion of the reaction, reaction mixture was concentrated under reduced pressure to give the crude residue, which was purified by silica gel chromatography using 10% methanol and DCM to afford Example 26 (21.4 mg; 0.04 mmol; 17.5%; purified product)

Yield: 21.4 mg (0.04 mmol), % Yield: 17.5.

### Analytical Data:

HPLC: Column: XBridge C8, 3.5 μm, 4.6×50 mm; Solvent A: water+0.1% TFA; Solvent B: ACN+0.1% TFA; Flow: 2 ml/min; Gradient: 0 min: 5% B, 8 min: 100% B, 8.1 min: 100% B, 8.5 min: 5% B, 10 min 5% B.

RT (min): 1.96; Purity (max): 97.4%; M+H=529.20.

¹H-NMR (400 MHz, DMSO-d6): δ 8.58 (d, J=8.00 Hz, 1H), 7.81 (d, J=7.60 Hz, 1H), 7.71 (t, J=15.60 Hz, 1H), 7.54 (t, J=15.60 Hz, 1H), 5.03 (s, br, 3H), 4.70 (s, br, 3H), 3.55-3.57 (m, 15H), 3.20 (s, br, 4H), 2.51-2.52 (m, 3H), 2.11 (s, 3H), 1.14-1.22 (m, 9H).

### Chemical Synthesis of Example 27

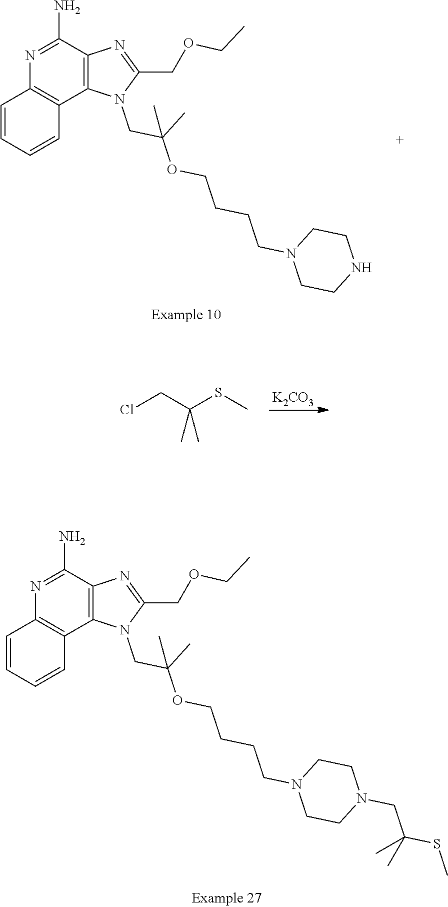

To a solution of Example 10 (100.00 mg; 0.22 mmol; 1.00 eq.) dissolved in acetonitrile (2.00 ml) 1-chloro-2-methyl-2-(methylsulfanyl) propane (91.50 mg; 0.66 mmol; 3.00 eq.) and potassium carbonate (46.53 mg; 0.33 mmol; 1.50 eq.) were added and stirred at 50° C. overnight. The reaction was monitored by TLC. After completion of the reaction, the reaction mixture was diluted with ethyl acetate and washed with water and the organic layer was dried with sodium sulphate, filtered and reduced under pressure to get crude residue. The crude residue was purified by using preparative HPLC using 0.1% TFA in water and ACN to afford Example 27 (5.80 mg; 0.01 mmol; 4.7%; purified product).

Yield: 5.80 mg (0.01 mmol), % Yield: 4.7.

### Analytical Data:

LCMS-COLUMN: ATLANTIS dC18 (50×4.6 mm) 5 μm; Mobile Phase: A 0.1% HCOOH in H2O:ACN (95:5), B: ACN Flow Rate: 1.5 ml/min.

RT (min): 2.00; Purity (max): 99.48%; M+H: 557.40.

### 6.2. Biological Evaluation of TLR7 Agonist

### 6.2.1 TLR7/8 Reporter Assays

### Material and General Procedure

HEK-Blue™ TLR7 and TLR8 cell lines were obtained from InvivoGen (San Diego, CA, USA). HEK-Blue cells were cultured in DMEM medium hi glucose (Gibco-10569010) supplemented with 10% HI-FBS (Aus origin) (Gibco-10100-147), 1% penicillin/streptomycin (Gibco-15140-122-100 ml) as per manufacturer's recommendation. In addition, specific antibiotic mixtures were added to the culture. 100 μg/ml Zeocin (11006-33-0), 10 μg/ml Blasticidin (ant-bl-05), 50 μg/ml Normocin (ANT-NR-1-500 MG) for HEK-Blue™ hTLR7 and 100 μg/ml Zeocin, 30 μg/ml Blasticidin, 50 μg/ml Normocin for HEK-Blue™ hTLR8 cells (all from InvivoGen).

### General Procedure for Screening Agonists In Vitro

All the compounds were prepared in 100% DMSO (Sigma-D2650). The compounds were serially diluted in 100% DMSO (3 fold) up to 10 point dose response curve starting with 30 μM; final concentration of DMSO in the plate was 0.3%. Resiquimod and TL8-506 (InvivoGen-tlrl-t18506) were used as controls. 0.3% DMSO and 10 μM Resiquimod were used as min and max respectively, and % activation was calculated using following formula:

% Activation: {100−(max absorbance−absorbance)/(max absorbance−min absorbance)}×100.

For stimulation with TLR7/8 agonists, cells were resuspended in complete media and 40 μl of cell suspension (10,000 cells/well) was seeded on day 1 in 384 well assay plate (Thermo Scientific™ Nunc-164688) having pre-dispensed compounds with Echo Liquid handler. The plate was spun at 100 g for 1 min to settle down the cells and incubated at 37° C. with 5% CO₂ for 24 hours. For measuring production of SEAP after 24 hrs of incubation, 10 μl of Quanti-Blue™ solution (InvivoGen rep-qbs2) prepared according to manufacturer's instructions was added in dark to the assay plate and incubated at 37° C. with 5% CO₂ for 30 min. Post incubation, the absorbance was measured at 620 nm in Tecan spark (Spark control Magellan). EC₅₀ were determined using Graph Pad Prism Statistical software (Version 7.05), with individual data points represent the absorbance values of duplicates. Table 1 describes compounds of the disclosure with an EC₅₀ value of less than 0.01 μM as “A” activity, between 0.01 μM and 0.1 μM as “B” activity, between 0.1 μM and 1 μM as “C” activity, between 1 μM and 10 μM as “D” activity and more than 10 μM as “E” activity.

TABLE 1
In vitro screening of TLR7/8 agonists in HEKBlue cells

| Compound # | TLR7 EC₅₀ [μM] | TLR8 EC₅₀ [μM] |
|---|---|---|
| Benchmark 1 | C | C |
| Benchmark 2 | E | E |
| Benchmark 3 | D | E |
| Benchmark 4 | D | E |
| Benchmark 5 | C | E |
| Benchmark 6 | B | B |
| Benchmark 7 | B | D |
| Example 1 | A | D |
| Example 2 | B | D |
| Example 3 | A | D |
| Example 4 | A | C |
| Example 5 | A | C |
| Example 6 | B | D |
| Example 7 | B | C |
| Example 8 | B | D |
| Example 9 | B | C |
| Example 10 | B | D |
| Example 11 | C | D |
| Example 12 | B | D |
| Example 13 | B | E |
| Example 14 | B | D |
| Example 15 | B | D |
| Example 16 | C | D |
| Example 17 | C | C |
| Example 18 | D | E |
| Example 19 | C | B |
| Example 20 | E | E |
| Example 21 | C | E |
| Example 22 | C | E |
| Example 23 | C | E |
| Example 24 | C | C |
| Example 25 | D | E |
| Example 26 | C | D |
| Example 27 | B | D |

In general, most of our examples have much higher potent on the TLR7 than the benchmarks. Moreover, our examples

6.2.2 TNFα Secretion from PBMCs

### Material and General Procedure

Peripheral blood mononuclear cells (PBMC) were isolated from healthy human volunteers and banked in liquid nitrogen. Vial of frozen PBMC was thawed in 37° C. water bath for no more than 2 minutes. The contents of the tube were aseptically transferred to pre-warmed complete media containing RPMI basal media (Gibco-21875034) supplemented with 10% HI-FBS (Aus origin) (Gibco-10100-147) and 1% penicillin/streptomycin (Gibco-15140-122-100 ml) followed by centrifugation. Cells were resuspended in complete media (3 ml) and counted using haemocytometer.

### General Procedure for Screening Agonists In Vitro

All selected compounds (Five benchmark and 15 example molecules) were prepared in 100% DMSO (Sigma-D2650) (10 mM stock). The compounds were serially diluted in 100% DMSO (4 fold) up to 8 point DRC in U-bottom 96 well plate (Cellstar-650180). 3 μl of this serially diluted DRC was mixed with 97 μl of completed media (1:33.33) as intermediate dilution (300 μM) 3% DMSO. 11 μl of compounds from intermediate dilution was added to the PBMC plate (1:10) such that final concentration of DMSO in the culture plate is 0.3% and starting concentration of the compounds is 30 μM. Resiquimod (30 μM) and DMSO (0.3%) were used as positive and negative controls respectively.

The plate was incubated at 37° C. with 5% CO₂ for 18 hours. Post incubation, the plate was spun at 2000 rpm for 5 min and supernatant was gently aspired and collected in protein low binding V-bottom 96 well plates (Thermo Fisher-249944). The supernatant plates were sealed, labelled appropriately and stored at −80° C. till cytokine analysis (<1 month).

MILLIPLEX MAP Human Cytokine/Chemokine Magnetic Bead Panel kit (HCYTOMAG-60K) was used for measuring TNF alpha as per the kit's recommendation.

EC₅₀ were determined using Graph Pad Prism Statistical software (Version 7.05), with individual data points represent the absorbance values of duplicates. Table 2 describes compounds of the disclosure with an EC50 value of less than 0.1 μM as “A” activity, between 0.1 μM and 1 μM as “B” activity, between 1 μM and 10 μM as “C” activity, and more than 10 μM as “D” activity.

TABLE 2
In vitro screening of selected TLR7/8 agonists in PBMCs

| Compound # | TNFα EC₅₀ [μM] |
|---|---|
| Benchmark 1 | C |
| Benchmark 2 | D |
| Benchmark 3 | C |
| Benchmark 5 | C |
| Benchmark 7 | D |
| Example 2 | B |
| Example 3 | A |
| Example 4 | A |
| Example 7 | B |
| Example 8 | C |
| Example 9 | D |
| Example 10 | B |
| Example 11 | C |
| Example 13 | B |
| Example 14 | B |
| Example 18 | C |
| Example 19 | A |
| Example 27 | C |

### 6.2.3 Expression of the Activation Marker CD86 in PBMCs

### Material and General Procedure

PBMCs were commercially procured from HemaCare Corporation and cultured in RPMI (Gibco-11875093) containing 10% HI-FBS. For stimulation, 50 μl of cell suspension containing 2λ10\hat{ }5 cells was seeded in the 96 well assay plate flat bottom white lid (Greiner Bio one-655180). Agonists were screened using freshly cultured PBMCs. FITC anti-human CD14 (Bioscience, 11-0149-42), PE anti-human CD123 (Biolegend, 396704), BV421 anti-human CD11c (Biolegend 301628) and APC anti-human CD86 (Biolegend, 374208) antibodies were used for flow cytometry staining.

General Procedure for Testing Agonists In Vitro All the compounds were made at 2-fold higher concentration to yield an intermediate stock (2 μM) using culture media. The compounds were serially diluted in culture media using 10-fold dilution for a total of 4-point DRC (2-0.002 μM). The intermediate stock (50 μl) and the serially diluted stock were added to the assay plate to gain a final concentration of 1-0.001 μM. Unstimulated cells or stimulated with 0.02% DMSO were used as control. The plate was incubated at 37° C. at 5% CO₂ for 21 hours.

The next day, cells were harvested using ice cold FACS buffer (DPBS w/o cations and containing 2% HI-FBS) and transferred to a separate 96 well V-bottom plate (Greiner, 650201). The cells were washed once using FACS buffer by spinning at 1400 rpm for 2 minutes at 4° C. A subsequent washing was done in PBS followed by live-dead staining of the cells using a zombie yellow stain (Biolegend, 423103; 1:100) diluted in PBS as per manufacturer protocol. The cells were washed twice in FACS buffer and stained using anti-CD14 FITC, anti-CD11c BV421, anti-CD123 PE and anti-CD86 APC antibodies (2 μl/test of each antibody) in FACS buffer for 1 hour at 4° C. in dark conditions. Following staining, the cells were washed twice using FACS buffer at 1400 rpm for 2 minutes at 4° C. The cells suspended in FACS buffer (100 μl/well) were analyzed using ACEA NovoCyte flow cytometer after setting up a compensation matrix using compensation beads except for live-dead stain for which the PBMCs stained with zombie yellow dye were used.

The PBMCs were gated using FSC-H and SSC-H which then were further gated for single cells using FSC-H and FSC-A. The singlets were gated for viability using live-dead stain captured in pacific orange channel. The viable singlets were gated for CD14+ (monocytes) population, CD14-CD11c+ (cDC population) and CD14-CD11c-CD123+ (pDC) population. Each population was further assessed for CD86+ expression in the respective channel. The negative and positive gates was assigned using respective FMO control using NovoExpress Software. An X-Y scatter plot was generated for each stimulant by plotting concentration vs median fluorescence intensity of CD86 stain within cDC population (FIG. 4A) and pDC population (FIG. 4B) and monocytes (FIG. 4C) using Graph Pad Prism9 software.

1. A compound according to formula I 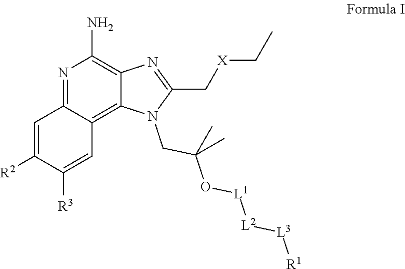
or a pharmaceutically acceptable salt thereof,
wherein X is an oxygen atom, C1-C5-alkyl or NH;
wherein R² and R³ are each independently selected from the group consisting of hydrogen, a C1-C5-alkyl, a C4-C7-cycloalkyl, a C4-C7-heterocycloalkyl, aryl and heteroaryl;
and wherein L¹, L², L³ and R¹ are as defined below under (a) or (b) or (c):
(a) L¹ is a C2-C6 alkyl;
L² is a 5- or 6-membered heterocycle or OH;
L³ is absent or selected from the group consisting of hydrogen, a C1-C2 alkyl and CH₂C(CH₃)₂; and
R₁ is absent or selected from the group consisting of hydrogen, NH₂, OH and SCH₃;
(b) L¹ is a C2-C6 alkyl;
L² is selected from the group consisting of C(O), NHC(O)CH₂ and NHC(O)C(CH₃)₂;
L³ is selected from the group consisting of a 6-membered heterocycle, OH, C(O)O, S, SO₂ and SO₃H; and
R¹ is absent or methyl or hydrogen;
(c) L¹ is C(O)CH₂;
L² is a 6-membered heterocycle or NH₂;
L³ is not present; and
R¹ is methyl or hydrogen or absent;
and wherein, if L³ is not present, then L² is directly bound via a covalent bond to R¹.

2. The compound according to claim 1, wherein R² and R³ are hydrogen.

3. The compound according to claim 1, wherein X is an oxygen atom.

4. The compound according to claim 3, wherein the compound has a structure according to formula II 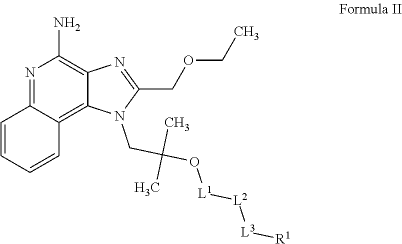
wherein L¹, L², L³ and R¹ are as defined below under (d), (e) or (f)
(d) L¹ is a C2-C6 alkyl;
L² is a 5- or 6-membered heterocycle, or OH; L³ is absent or selected from the group consisting of hydrogen, a C1-C2 alkyl and CH₂C(CH₃)₂; and
R¹ is absent or selected from the group consisting of hydrogen, NH₂, OH and SCH₃;
(e) L¹ is a C2-C3 alkyl;
L² is selected from the group consisting of C(O), NHC(O)CH₂ and NHC(O)C(CH₃)₂;
L³ is selected from the group consisting of a 6-membered heterocycle, OH, C(O)O, S, SO₂ and SO₃H; and
R¹ is absent or methyl or hydrogen;
(f) L¹ is C(O)CH₂;
L² is a 6-membered heterocycle or NH₂;
L³ is not present; and
R¹ is methyl or hydrogen or absent;

5. The compound according to claim 4, wherein
L¹ is a C2-C6 alkyl;
L² is a 5- or 6-membered heterocycle or OH;
L³ is absent or selected from the group consisting of hydrogen, a C1-C2 alkyl and CH₂C(CH₃)₂; and
R¹ is absent or selected from the group consisting of NH₂, OH and SCH₃.

6. The compound according to claim 1, wherein the compound has a structure selected from the group consisting of the structures listed below that are designated as Example 1 through Example 27:

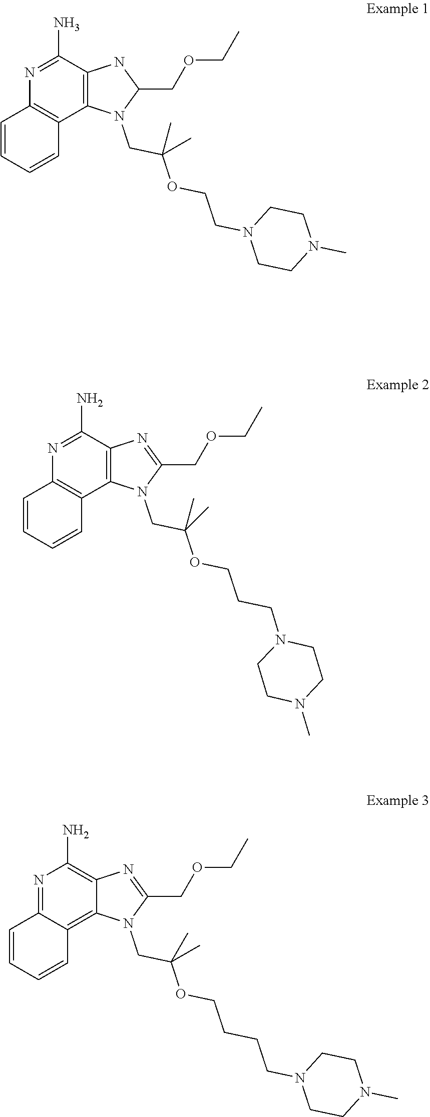 [SMILES: CCOCC1Nc2c(N)nc3ccccc3c2N1CC(C)(C)OCCN1CCN(C)CC1 ; CCOCc1nc2c(N)nc3ccccc3c2n1CC(C)(C)OCCCN1CCN(C)CC1 ; CCOCc1nc2c(N)nc3ccccc3c2n1CC(C)(C)OCCCCN1CCN(C)CC1]

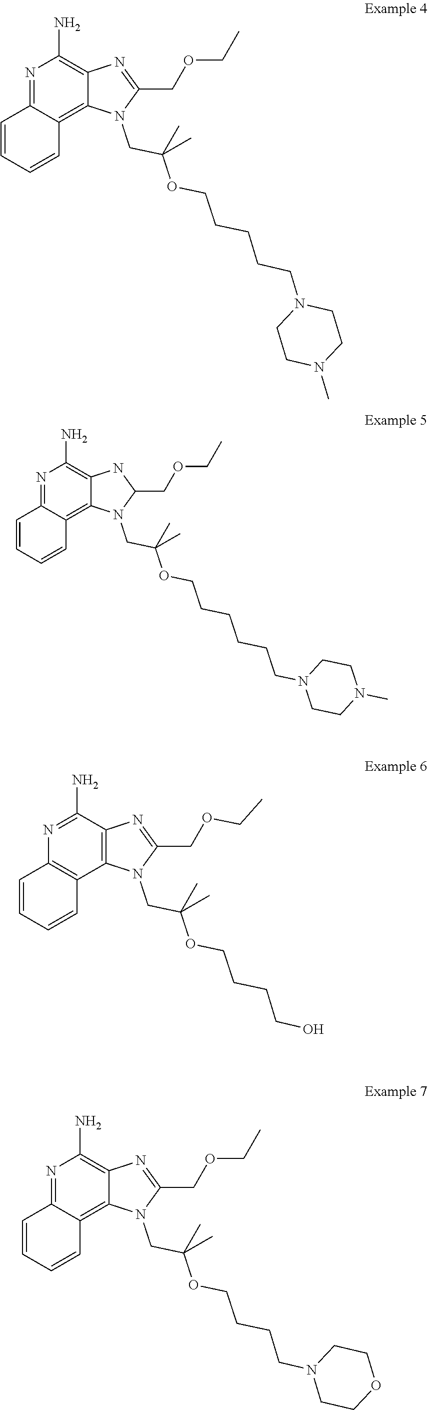 [SMILES: CCOCc1nc2c(N)nc3ccccc3c2n1CC(C)(C)OCCCCCN1CCN(C)CC1 ; CCOCC1Nc2c(N)nc3ccccc3c2N1CC(C)(C)OCCCCCCN1CCN(C)CC1 ; CCOCc1nc2c(N)nc3ccccc3c2n1CC(C)(C)OCCCCO ; CCOCc1nc2c(N)nc3ccccc3c2n1CC(C)(C)OCCCCN1CCOCC1]

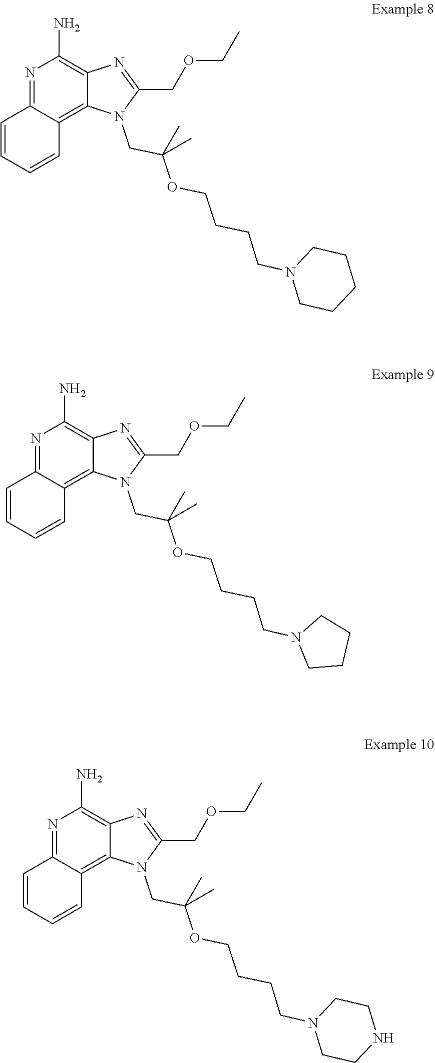 [SMILES: CCOCc1nc2c(N)nc3ccccc3c2n1CC(C)(C)OCCCCN1CCCCC1 ; CCOCc1nc2c(N)nc3ccccc3c2n1CC(C)(C)OCCCCN1CCCC1 ; CCOCc1nc2c(N)nc3ccccc3c2n1CC(C)(C)OCCCCN1CCNCC1]

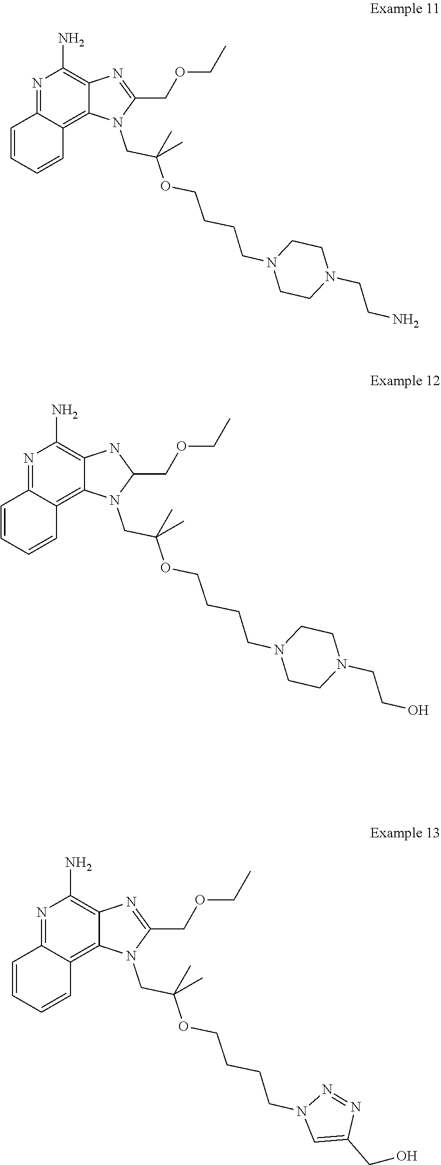 [SMILES: CCOCc1nc2c(N)nc3ccccc3c2n1CC(C)(C)OCCCCN1CCN(CCN)CC1 ; CCOCC1Nc2c(N)nc3ccccc3c2N1CC(C)(C)OCCCCN1CCN(CCO)CC1 ; CCOCc1nc2c(N)nc3ccccc3c2n1CC(C)(C)OCCCCn1cc(CO)nn1]

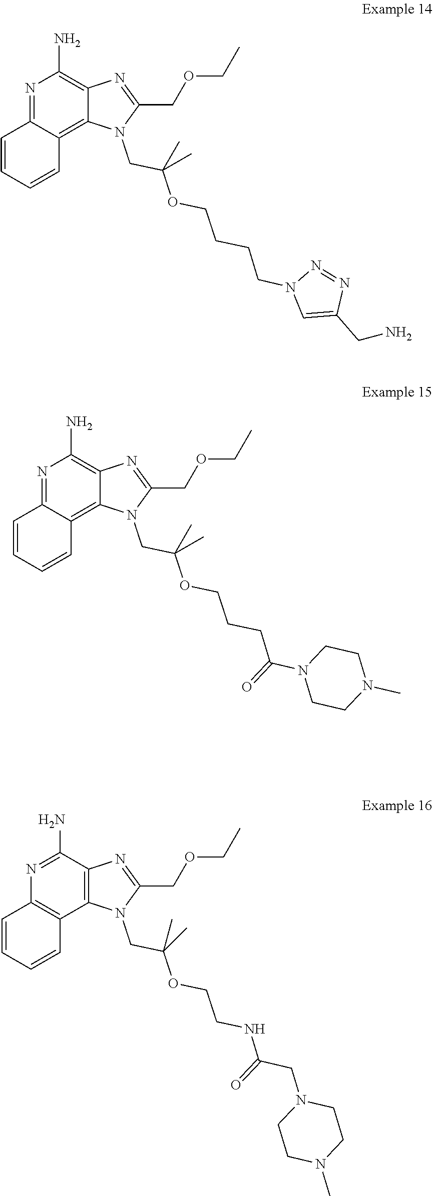 [SMILES: CCOCc1nc2c(N)nc3ccccc3c2n1CC(C)(C)OCCCCn1cc(CN)nn1 ; CCOCc1nc2c(N)nc3ccccc3c2n1CC(C)(C)OCCCC(=O)N1CCN(C)CC1 ; CCOCc1nc2c(N)nc3ccccc3c2n1CC(C)(C)OCCNC(=O)CN1CCN(C)CC1]

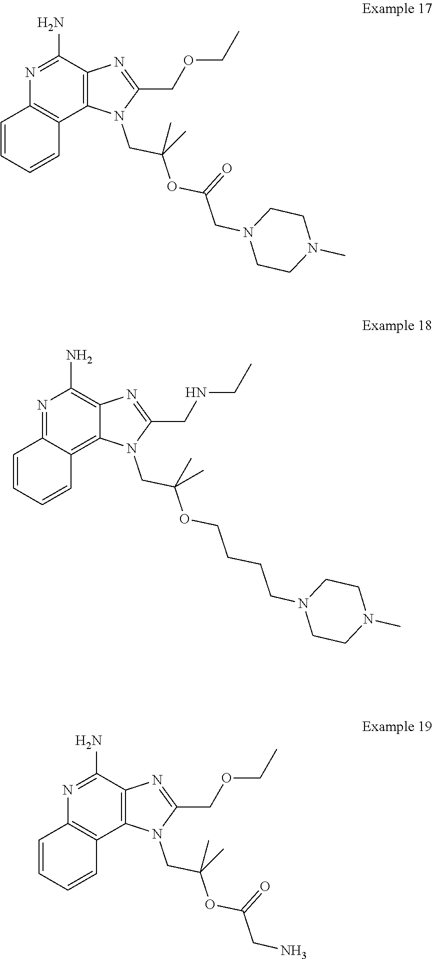 [SMILES: CCOCc1nc2c(N)nc3ccccc3c2n1CC(C)(C)OC(=O)CN1CCN(C)CC1 ; CCNCc1nc2c(N)nc3ccccc3c2n1CC(C)(C)OCCCCN1CCN(C)CC1 ; CCOCc1nc2c(N)nc3ccccc3c2n1CC(C)(C)OC(=O)CN]

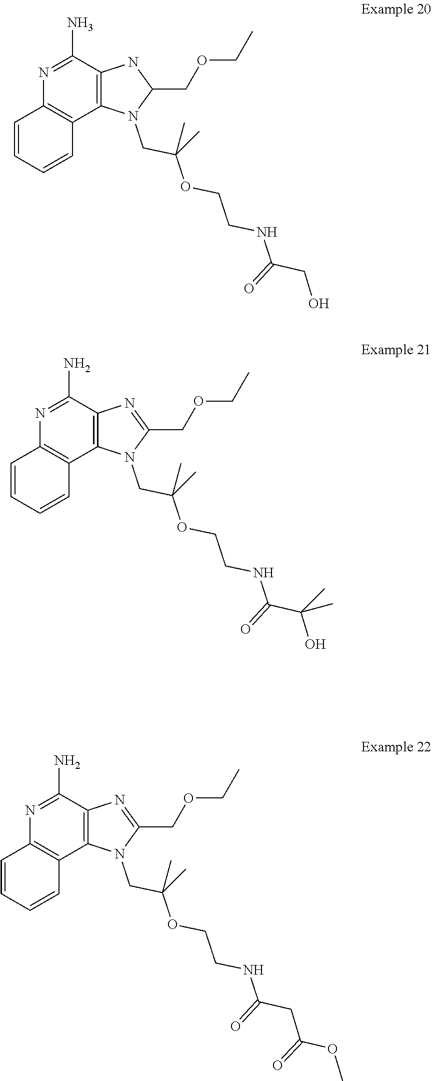 [SMILES: CCOCC1Nc2c(N)nc3ccccc3c2N1CC(C)(C)OCCNC(=O)CO ; CCOCc1nc2c(N)nc3ccccc3c2n1CC(C)(C)OCCNC(=O)C(C)(C)O ; CCOCc1nc2c(N)nc3ccccc3c2n1CC(C)(C)OCCNC(=O)CC(=O)OC]

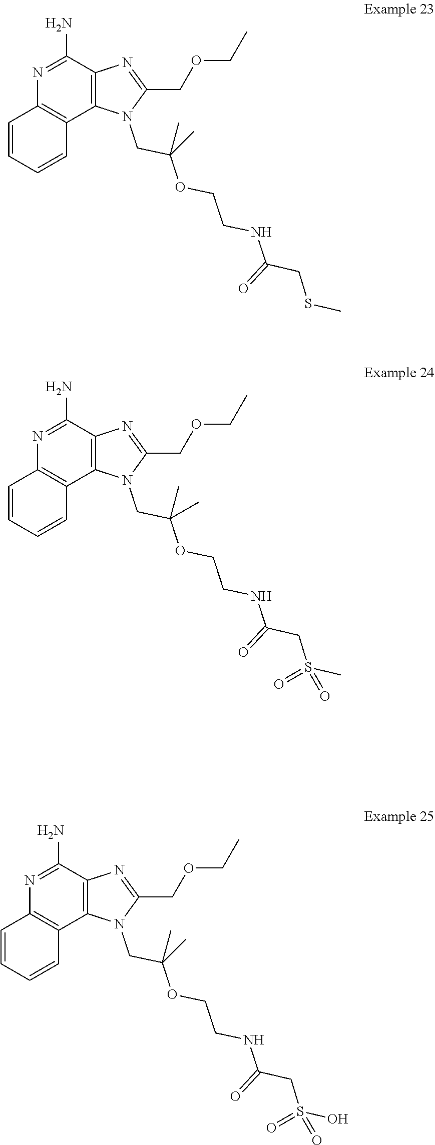 [SMILES: CCOCc1nc2c(N)nc3ccccc3c2n1CC(C)(C)OCCNC(=O)CSC ; CCOCc1nc2c(N)nc3ccccc3c2n1CC(C)(C)OCCNC(=O)CS(C)(=O)=O ; CCOCc1nc2c(N)nc3ccccc3c2n1CC(C)(C)OCCNC(=O)CS(=O)(=O)O]

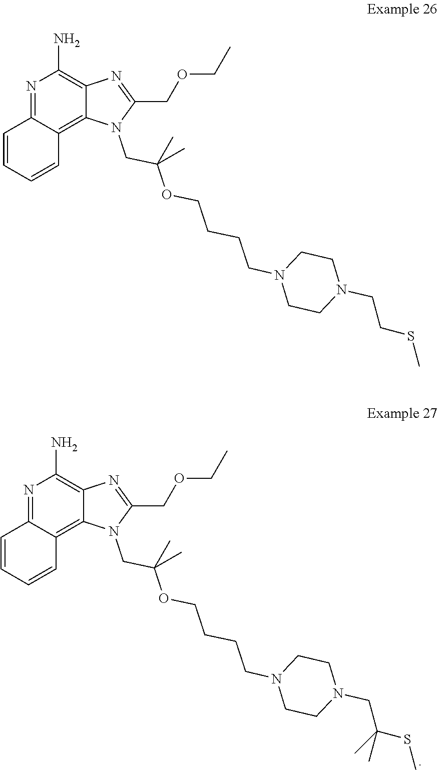 [SMILES: CCOCc1nc2c(N)nc3ccccc3c2n1CC(C)(C)OCCCCN1CCN(CCSC)CC1 ; CCOCc1nc2c(N)nc3ccccc3c2n1CC(C)(C)OCCCCN1CCN(CC(C)(C)SC)CC1]

7. The compound according to claim 1, wherein
X is NH;
L¹ is a C2-C6 alkyl;
L² is a 5- or 6-membered heterocycle;
L³ is not present; and
R¹ is methyl or hydrogen; and
R² and R³ are each hydrogen.

8. The compound according to claim 1, wherein the compound
(i) activates TLR7 more strongly than TLR8; and/or
(ii) induces IL-6, IL1-b and TNF-α production; and/or
(iii) induces upregulation of CD40 and/or CD86 in peripheral blood mononuclear cells (PBMC).

9. The compound according to claim 1, wherein the compound induces TNFα secretion from peripheral blood mononuclear cells (PBMC) if the compound contacts the PBMC.

10. The compound according to claim 1, wherein the compound has for TLR7 an EC50 of less than 10 μM.

11. A pharmaceutical composition, comprising: a compound of claim 1.

12. A medicament, comprising:
the compound of claim 1

13. A method, comprising:
treating a disease with the compound of claim 1 in combination with further compounds Imiquimod and/or Resiquimod (R848).

14. A composition, comprising:
the compound of claim 1,
for use in the treatment of a condition selected from the group consisting of cancer, a viral infection, non-cancerous skin lesions, pre-cancerous skin lesions, cancerous skin lesions, bladder cancer, and virally mediated skin disease,
said composition optionally being formulated for enhanced penetration following topical administration.

15. The pharmaceutical composition of claim 11, comprising:
between 0.01%-1% (wt/vol) of the compound.

16. The composition of claim 14 for use in the treatment of bladder cancer, wherein the composition is formulated for intracystic administration.

17. The pharmaceutical composition of claim 11, further comprising:
additional components of one of the groups i through xiii outlined below:
i. oleic acid (50%) and isopropyl myristate (50%) in a solution containing 50 ml phosphate buffered saline and 50 ml ethanol at a total concentration of 1% wt/vol, or
ii. isopropyl myristate (50%) and sodium lauryl sulfate (50%) in a solution containing 50 ml phosphate buffered saline and 50 ml ethanol at a total concentration of 0.5% wt/vol, or
iii. oleic acid (50%) and sodium lauryl sulfate (50%) in a solution containing 50 ml phosphate buffered saline and 50 ml ethanol at a total concentration of 0.5% wt/vol, or
iv. oleic acid (33%), isopropyl myristate (33%) and sodium lauryl sulfate (33%) in a solution containing 50 ml phosphate buffered saline and 50 ml ethanol at a total concentration of 0.5% wt/vol, or
v. isopropyl palmitate (50%) and sodium oleate (50%) in a solution containing 50 ml phosphate buffered saline and 50 ml ethanol at a total concentration of 2% wt/vol, or
vi. sodium lauryl sulfate (25%) and linoleic acid (75%) in a solution containing 50 ml phosphate buffered saline and 50 ml ethanol at a total concentration of 1.0% wt/vol, or
vii. palmitic acid (50%) and isopropyl laurate (50%) in a solution containing 50 ml phosphate buffered saline and 50 ml ethanol at a total concentration of 2.0% wt/vol, or
viii. oleic acid (50%) and linoleic acid (50%) in a solution containing 50 ml phosphate buffered saline and 50 ml ethanol at a total concentration of 1.5% wt/vol, or
ix. linoleic acid (25%), oleic acid (25%) and isopropyl linoleate (50%) in a solution containing 50 ml phosphate buffered saline and 50 ml ethanol at a total concentration of 0.5% wt/vol, or
x. sodium oleate (33%), oleic acid (33%) and methyl palmitate (33%) in a solution containing 50 ml phosphate buffered saline and 50 ml ethanol at a total concentration of 2.0% wt/vol, or
xi. a solution containing 50 ml phosphate buffered saline and 50 ml ethanol, or
xii. oleic acid (10%) in a solution containing 50 ml phosphate buffered saline and 50 ml ethanol, or
xiii. oleic acid (2%) and sodium lauryl sulfate (5%) in a solution containing 50 ml phosphate buffered saline and 50 ml ethanol.

18. A method for the treatment of a cancer, the method comprising:
administering a therapeutically effective amount of the compound of claim 1 to the subject suffering from cancer.

19. A method for the treatment of skin tumor lesion and virus induced skin disease, the method comprising:
topically administering the pharmaceutical composition of claim 11.

20. A method for the treatment of a bladder tumor, the method comprising:
intracystically administering the composition of claim claim 11.

21. The method of claim 18, the method further comprising:
systemically administering at least one immune modulator selected from the group consisting of anti-PD1, anti-PD-L1, Anti-CTLA-4 antibody, anti-CD137 antibody, agonistic CD40 antibody, CD134 (Anti-OX40) agonist and PLX3397.

22. The method of claim 18, further comprising:
systemically administering interferon gamma.

23. The method of claim 18, further comprising: administering local radiation with or without systemic anti-PD1 antibody.

24. The method of claim 18, further comprising:
administering photodynamic therapy.

25. A method of activating TLR 7 and/or 8 in a biological sample, the method comprising:
contacting the biological sample with a compound according to claim 1.

26. A vaccine adjuvant, comprising:
the compound claim 1.
